SEQUENCE LISTING (12 sequences)

SEQ ID NO: 1 - DNA, 12 bp, synthetic construct
  source 1..12: mol_type = other DNA
ttcttcttcttc

SEQ ID NO: 2 - DNA, 15 bp, synthetic construct
  source 1..15: mol_type = other DNA
ttcttcttcttcttc

SEQ ID NO: 3 - DNA, 18 bp, synthetic construct
  source 1..18: mol_type = other DNA
ttcttcttcttcttcttc

SEQ ID NO: 4 - DNA, 21 bp, synthetic construct
  source 1..21: mol_type = other DNA
ttcttcttcttcttcttcttc

SEQ ID NO: 5 - DNA, 12 bp, synthetic construct
  source 1..12: mol_type = other DNA
gaagaagaagaa

SEQ ID NO: 6 - DNA, 15 bp, synthetic construct
  source 1..15: mol_type = other DNA
gaagaagaagaagaa

SEQ ID NO: 7 - DNA, 18 bp, synthetic construct
  source 1..18: mol_type = other DNA
gaagaagaagaagaagaa

SEQ ID NO: 8 - DNA, 21 bp, synthetic construct
  source 1..21: mol_type = other DNA
gaagaagaagaagaagaagaa

SEQ ID NO: 9 - RNA, 12 nt, synthetic construct
  source 1..12: mol_type = other RNA
cttcttcttctt

SEQ ID NO: 10 - RNA, 15 nt, synthetic construct
  source 1..15: mol_type = other RNA
cttcttcttcttctt

SEQ ID NO: 11 - RNA, 18 nt, synthetic construct
  source 1..18: mol_type = other RNA
cttcttcttcttcttctt

SEQ ID NO: 12 - RNA, 21 nt, synthetic construct
  source 1..21: mol_type = other RNA
cttcttcttcttcttcttctt

(19) United States
(12) Patent Application Publication
(10) Pub. No.: US 2025/0340567 A1
(43) Pub. Date: Nov. 6, 2025

(54) Next Generation Synthetic Transcription Factors

(71) Applicant: WISCONSIN ALUMNI RESEARCH FOUNDATION, Madison, WI (US)

(72) Inventors: Aseem Z. Ansari, Madison, WI (US); Ashraf Mohammed, Madison, WI (US)

(73) Assignee: WISCONSIN ALUMNI RESEARCH FOUNDATION, Madison, WI (US)

(21) Appl. No.: 19/221,144

(22) Filed: May 28, 2025

Related U.S. Application Data
(63) Continuation of application No. 17/287,901, filed on Apr. 22, 2021, now abandoned.
(60) Provisional application No. 62/749,567, filed on Oct. 23, 2018.

(51) Int. Cl. C07D 495/14; C07D 403/14; C07D 471/04

(52) U.S. Cl. CPC C07D 495/14; C07D 403/14; C07D 471/04

(57) ABSTRACT

Synthetic transcriptions factors for modulation of frataxin expression and methods of use are described herein.

### CROSS-REFERENCE TO RELATED APPLICATIONS

This application claims priority to U.S. Provisional Application No. 62/749,567, filed Oct. 23, 2018, which is incorporated herein in its entirety.

### STATEMENT REGARDING FEDERALLY SPONSORED RESEARCH

This invention was made with government support under GM117362 and TR002373 awarded by the National Institutes of Health. The government has certain rights in the invention.

### REFERENCE TO A SEQUENCE LISTING SUBMITTED VIA EFS-WEB

The content of the ASCII text file of the sequence listing named “960296_02536_ST25.txt” which is 2.42 kb in size was created on Oct. 22, 2019 and electronically submitted via EFS-Web herewith the application is incorporated herein by reference in its entirety.

### BACKGROUND

Friedreich's ataxia (also referred to as FA or FRDA) is a rare but fatal autosomal recessive neurodegenerative disease, with an estimated incidence of 1 in every 40,000 people. This condition is typically found in individuals with European, Middle Eastern, or North African ancestry. FRDA causes progressive damage to the nervous system and muscle cells, resulting in a loss of coordination as well as various neurological and cardiac complications. In particular, FRDA patients develop neurodegeneration of the large sensory neurons and spinocerebellar tracts, as well as cardiomyopathy and diabetes mellitus. Onset of symptoms is typically seen between the ages of 5 and 15 years, and the mean age of death is approximately 38 years.

Friedreich's ataxia is caused by an abnormal expansion of the guanine-adenine-adenine (GAA) trinucleotide repeat sequences in intron 1 of the frataxin (FXN) gene, resulting in transcriptional repression and reduced expression of the frataxin (FXN) protein. Frataxin, which is encoded by the nuclear frataxin (FXN) gene, is a highly-conserved, 210-amino acid protein that is localized to the mitochondrion. Most FRDA patients (approximately 98%) carry a homozygous mutation characterized by an expansion of a GAA trinucleotide repeat in the first intron of the frataxin (FXN) gene. Pathological GAA expansions can range from about 66 to more than 1,000 trinucleotide repeats, whereas frataxin alleles that are not associated with FRDA comprise from about 6 to about 34 repeats.

There is presently no cure for FRDA or specific therapy to prevent progression of the disease approved for use as a treatment. Therefore, there is a need to develop compositions that restore or partially restore frataxin levels to treat and/or prevent FRDA.

### SUMMARY

In a first aspect, provided herein is a synthetic transcription factor having formula A-L-B wherein -L- is a linker; —B is a nucleic acid binding moiety that specifically binds to one or more repeats of a GAA oligonucleotide sequence; and A- is a bromodomain binding moiety selected from the group consisting of I-BET762, I-BET726, BET-BAY002, CPI 0610, a bromodomain binding moiety having the structure of formula 7:

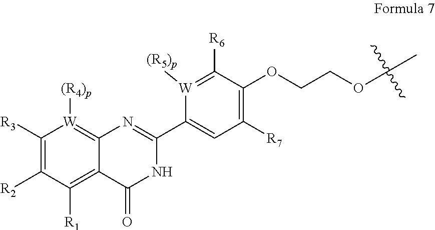

wherein R₁ and R₃ are each independently selected from alkoxy, alkyl, amino, halogen, and hydrogen; R₂ is selected from alkoxy, alkyl, alkenyl, alkynyl, amide, amino, halogen, and hydrogen; R₆ and R₇ are each independently selected from alkyl, alkoxy, amino, halogen, and hydrogen; R₅ is hydrogen; each W is independently selected from C and N, wherein if W is N, then p is 0 or 1, and if W is C, then p is 1; for W—(R₄)ₚ, if W is C, p is 1 and R₄ is H, or if W is N, then p is 0; if R₁ is hydrogen, then R₃ is alkoxy; if R₃ is hydrogen, then R₁ is selected from amino and alkoxy; and at least one of R₆ and R₇ is independently selected from alkyl, alkoxy, amino, and halogen, and a bromodomain binding moiety having the structure of formula 9:

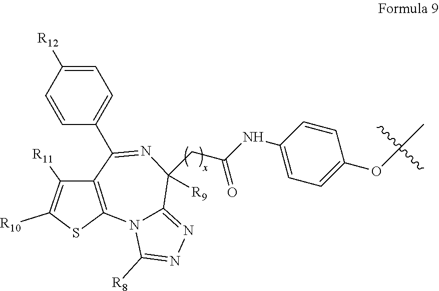

wherein R₈, R₁₀, and R₁₁ are each independently selected from hydrogen, methyl, ethyl, and halomethyl; R₉ is selected from hydrogen, C₁-C₆ alkyl group, and substituted C₁-C₆ alkyl group; R₁₂ is selected from halogen, aryl, substituted aryl, amino, and amido; and X is an integer from 1 to 6. In some embodiments, the bromodomain binding moiety has the structure of formula 10:

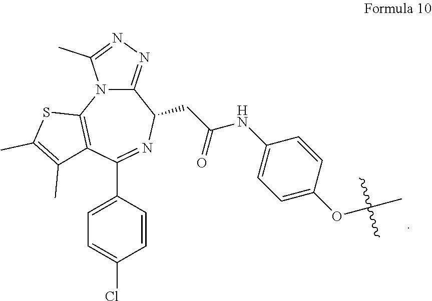 [SMILES: *Oc1ccc(NC(=O)C[C@@H]2N=C(c3ccc(Cl)cc3)c3c(sc(C)c3C)-n3c(C)nnc32)cc1]

In some embodiments, the bromodomain binding moiety has the structure of formula 8:

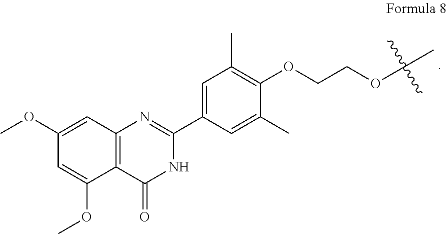 [SMILES: *OCCOc1c(C)cc(-c2nc3cc(OC)cc(OC)c3c(=O)[nH]2)cc1C]

In some embodiments, —B is a polyamide that specifically binds to one or more repeats of a GAA oligonucleotide. In some embodiments, —B comprises one or more of the following subunits:

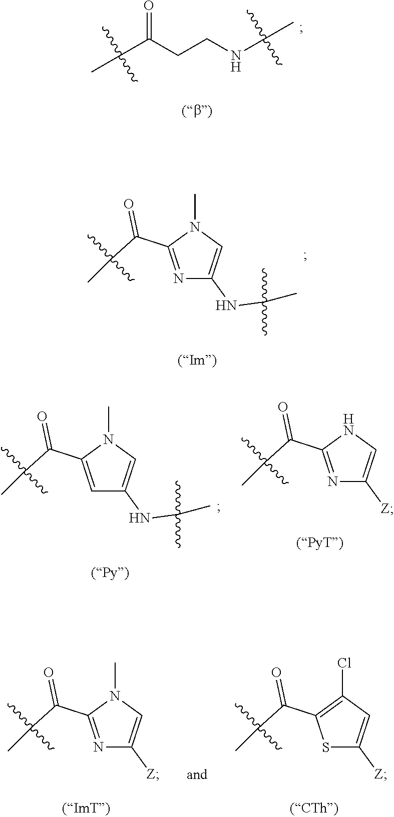

wherein Z is hydrogen, an amino, or an amido group. In some embodiments, —B is selected from the group consisting of PA1, PA5, PA14, PA16, PA17, and PA18, wherein PA1 has the structure of formula 1:

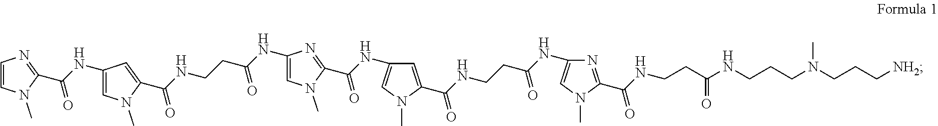 [SMILES: CN(CCCN)CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C]

wherein PA5 has the structure of formula 2:

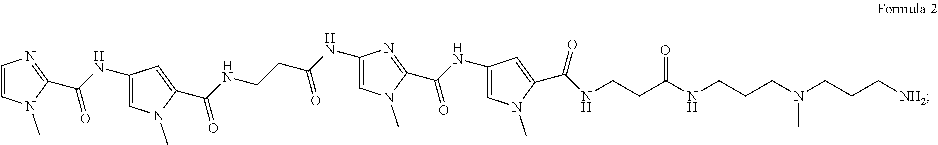 [SMILES: CN(CCCN)CCCNC(=O)CCNC(=O)c1cc(NC(=O)c2nc(NC(=O)CCNC(=O)c3cc(NC(=O)c4nccn4C)cn3C)cn2C)cn1C]

wherein PA14 has the structure of formula 3:

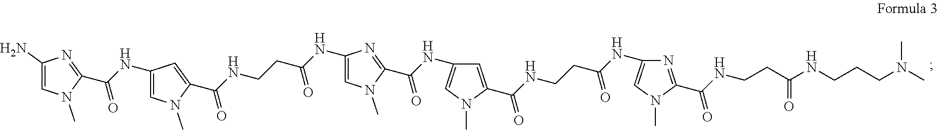 [SMILES: CN(C)CCCNC(=O)CCNC(=O)c1nc(NC(=O)CCNC(=O)c2cc(NC(=O)c3nc(NC(=O)CCNC(=O)c4cc(NC(=O)c5nc(N)cn5C)cn4C)cn3C)cn2C)cn1C]

wherein PA16 has the structure of formula 4:

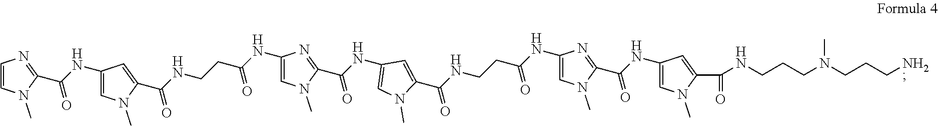 [SMILES: CN(CCCN)CCCNC(=O)c1cc(NC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)cn1C]

wherein PA17 has the structure of formula 5:

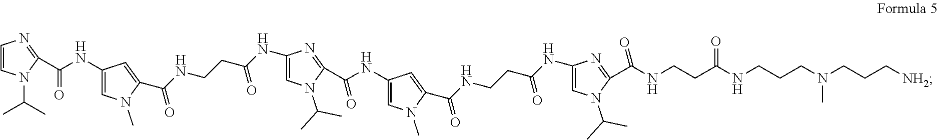 [SMILES: CC(C)n1cc(NC(=O)CCNC(=O)c2cc(NC(=O)c3nc(NC(=O)CCNC(=O)c4cc(NC(=O)c5nccn5C(C)C)cn4C)cn3C(C)C)cn2C)nc1C(=O)NCCC(=O)NCCCN(C)CCCN]

and wherein PA18 has the structure of formula 6:

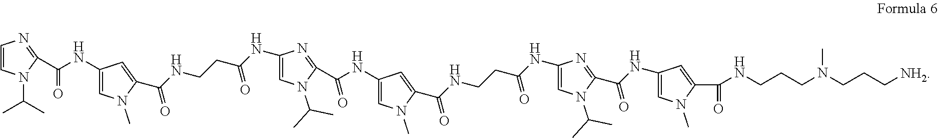 [SMILES: CC(C)n1ccnc1C(=O)Nc1cc(C(=O)NCCC(=O)Nc2cn(C(C)C)c(C(=O)Nc3cc(C(=O)NCCC(=O)Nc4cn(C(C)C)c(C(=O)Nc5cc(C(=O)NCCCN(C)CCCN)n(C)c5)n4)n(C)c3)n2)n(C)c1]

In some embodiments, the linker is a polyethylene glycol (PEG) linker. In some embodiments, the PEG linker is selected from the group consisting of (O—CH₂—CH₂)₂ (PEG₂), (O—CH₂—CH₂)₃ (PEG₃), (O—CH₂—CH₂)₄ (PEG₄), (O—CH₂—CH₂)₅ (PEG₅), (O—CH₂—CH₂)₆ (PEG₆), (O—CH₂—CH₂)₇ (PEG₇), (O—CH₂—CH₂)₈ (PEG₈), (O—CH₂—CH₂)₉ (PEG₉), or (O—CH₂—CH₂)₁₀ (PEG₁₀).

In some embodiments, the synthetic transcription factor has a non-amide bond between A- and -L-. In some embodiments, the synthetic transcription factor has an ether bond between A- and -L-. In some embodiments, the synthetic transcription factor has a non-amide bond between -L- and -B.

In some embodiments, the synthetic transcription factor has the structure of formula 35:

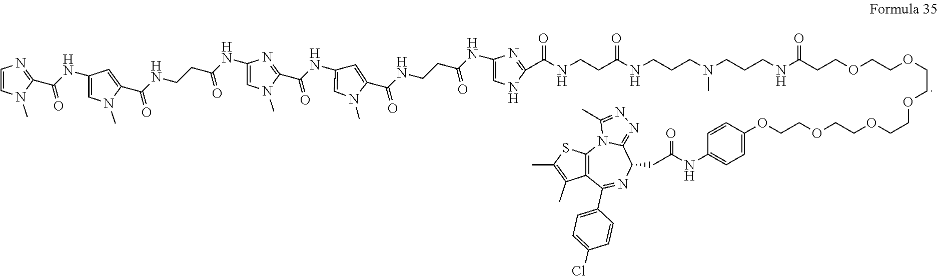 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)c[nH]3)cc1)c1nnc(C)n1-2]

In some embodiments, the synthetic transcription factor has the structure of formula 36:

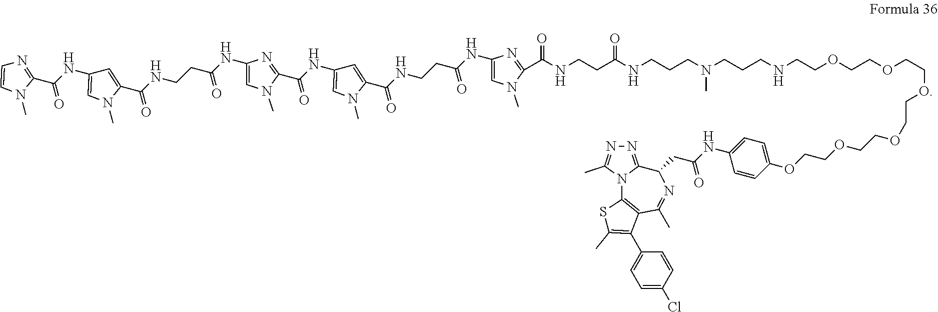 [SMILES: CC1=N[C@@H](CC(=O)Nc2ccc(OCCOCCOCCOCCOCCOCCNCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc2)c2nnc(C)n2-c2sc(C)c(-c3ccc(Cl)cc3)c21]

In some embodiments, the synthetic transcription factor has the structure of formula 37:

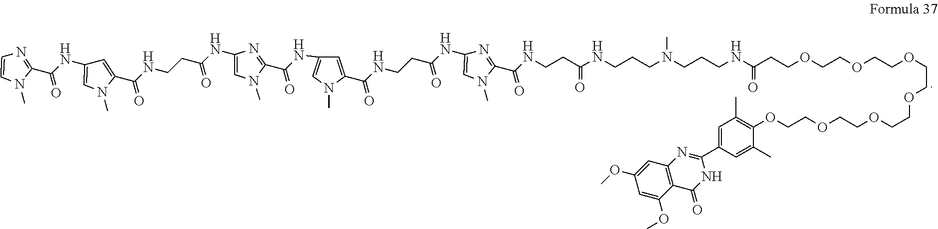 [SMILES: COc1cc(OC)c2c(=O)[nH]c(-c3cc(C)c(OCCOCCOCCOCCOCCOCCOCCC(=O)NCCCN(C)CCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(C)c3)nc2c1]

In some embodiments, the synthetic transcription factor has the structure of formula 38:

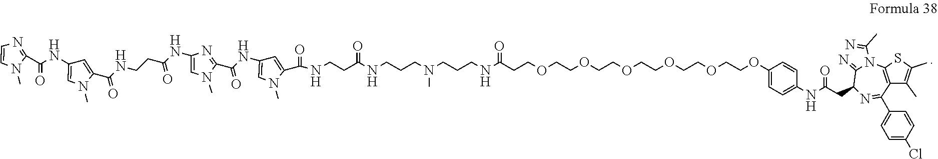 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)CCNC(=O)c3cc(NC(=O)c4nc(NC(=O)CCNC(=O)c5cc(NC(=O)c6nccn6C)cn5C)cn4C)cn3C)cc1)c1nnc(C)n1-2]

In some embodiments, the synthetic transcription factor has the structure of formula 41:

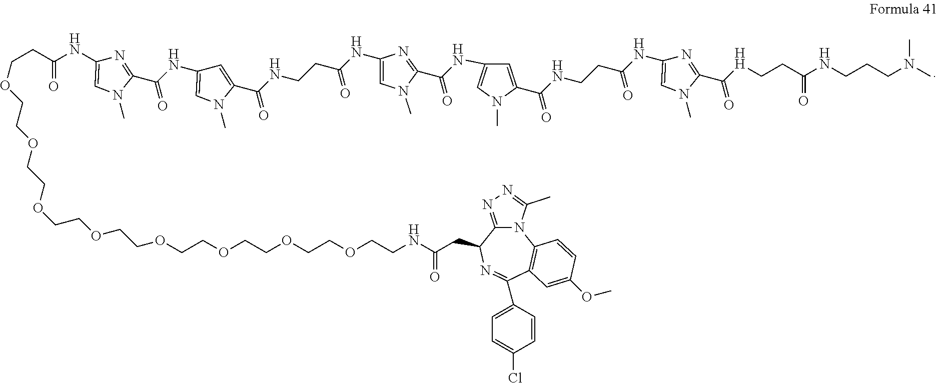

In some embodiments, the synthetic transcription factor has the structure of formula 42:

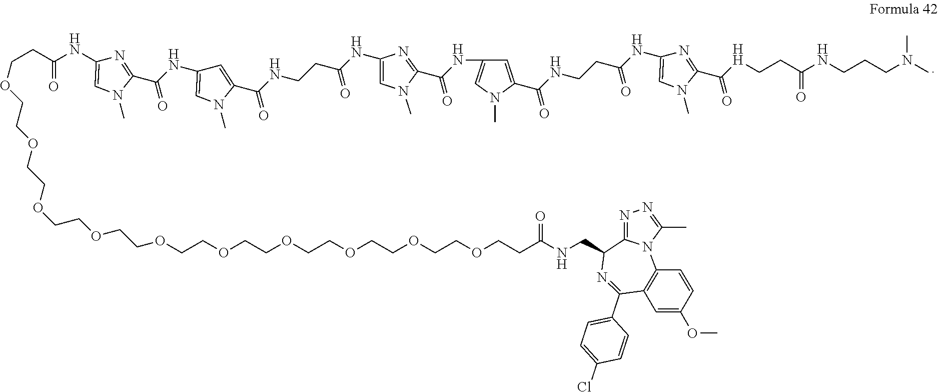

In some embodiments, the synthetic transcription factor has the structure of formula 44:

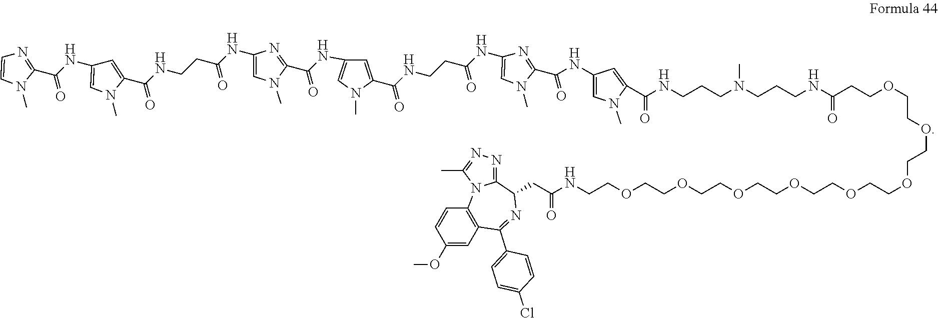 [SMILES: COc1ccc2c(c1)C(c1ccc(Cl)cc1)=N[C@@H](CC(=O)NCCOCCOCCOCCOCCOCCOCCOCCOCCC(=O)NCCCN(C)CCCNC(=O)c1cc(NC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cn1C)c1nnc(C)n1-2]

In some embodiments, the synthetic transcription factor has the structure of formula 46:

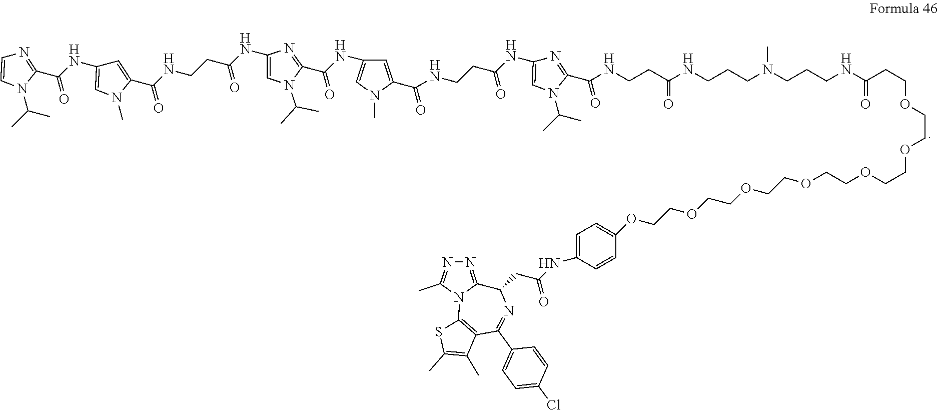 [SMILES: Cc1sc2c(c1C)C(c1ccc(Cl)cc1)=N[C@@H](CC(=O)Nc1ccc(OCCOCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C(C)C)cn6C)cn5C(C)C)cn4C)cn3C(C)C)cc1)c1nnc(C)n1-2]

In some embodiments, the synthetic transcription factor has the structure of formula 47:

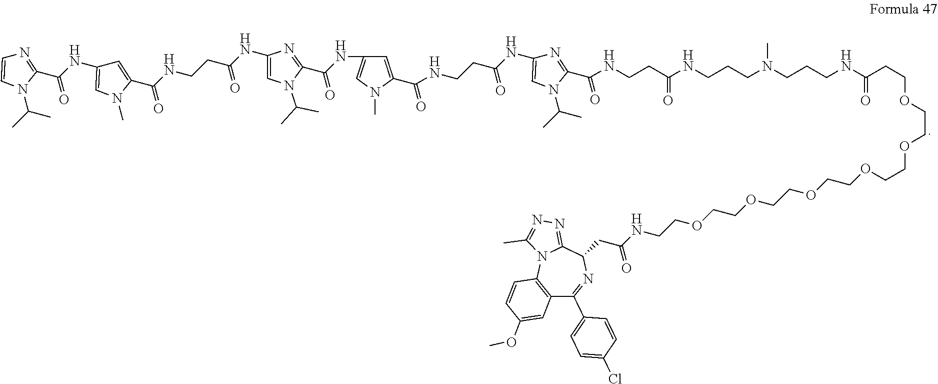 [SMILES: COc1ccc2c(c1)C(c1ccc(Cl)cc1)=N[C@@H](CC(=O)NCCOCCOCCOCCOCCOCCOCCC(=O)NCCCN(C)CCCNC(=O)CCNC(=O)c1nc(NC(=O)CCNC(=O)c3cc(NC(=O)c4nc(NC(=O)CCNC(=O)c5cc(NC(=O)c6nccn6C(C)C)cn5C)cn4C(C)C)cn3C)cn1C(C)C)c1nnc(C)n1-2]

In some embodiments, the synthetic transcription factor has the structure of formula 49:

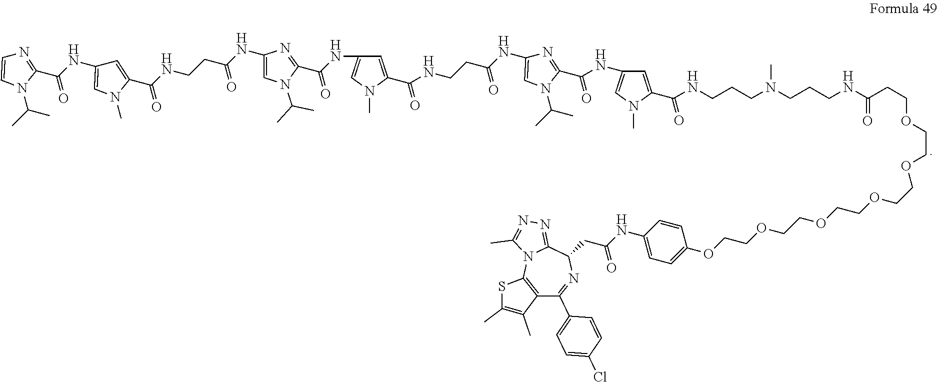 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)c3cc(NC(=O)c4nc(NC(=O)CCNC(=O)c5cc(NC(=O)c6nc(NC(=O)CCNC(=O)c7cc(NC(=O)c8nccn8C(C)C)cn7C)cn6C(C)C)cn5C)cn4C(C)C)cn3C)cc1)c1nnc(C)n1-2]

In some embodiments, the synthetic transcription factor has the structure of formula 50;

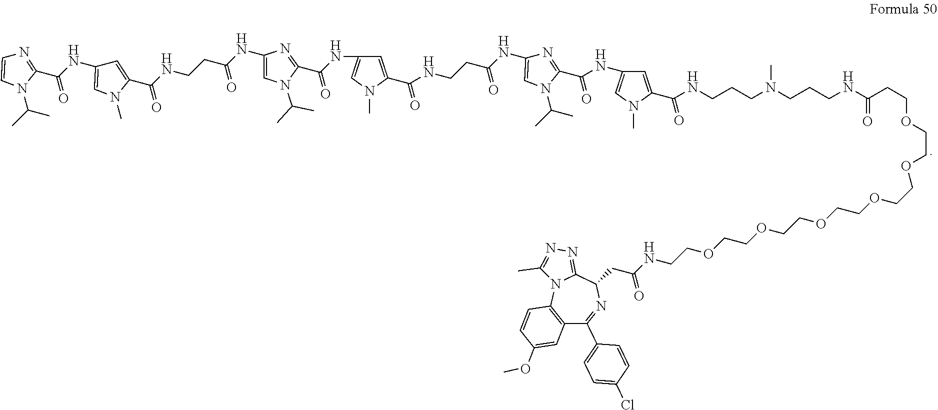 [SMILES: COc1ccc2c(c1)C(c1ccc(Cl)cc1)=N[C@@H](CC(=O)NCCOCCOCCOCCOCCOCCOCCC(=O)NCCCN(C)CCCNC(=O)c1cc(NC(=O)c3nc(NC(=O)CCNC(=O)c4cc(NC(=O)c5nc(NC(=O)CCNC(=O)c6cc(NC(=O)c7nccn7C(C)C)cn6C)cn5C(C)C)cn4C)cn3C(C)C)cn1C)c1nnc(C)n1-2]

In some embodiments, the synthetic transcription factor has the structure of formula 51:

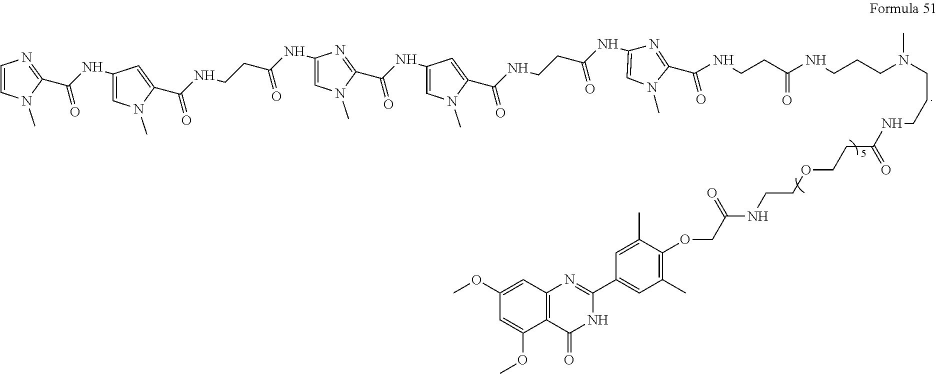

In some embodiments, the synthetic transcription factor has the structure of formula 52:

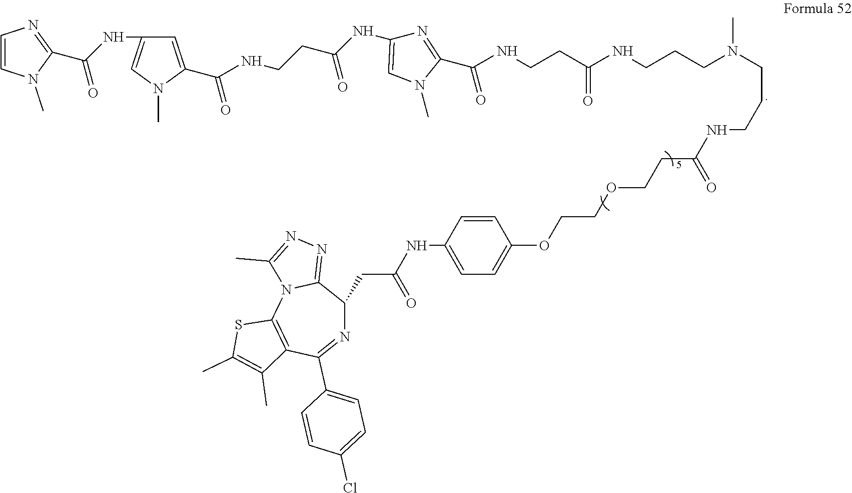

In some embodiments, the synthetic transcription factor has the structure of formula 53:

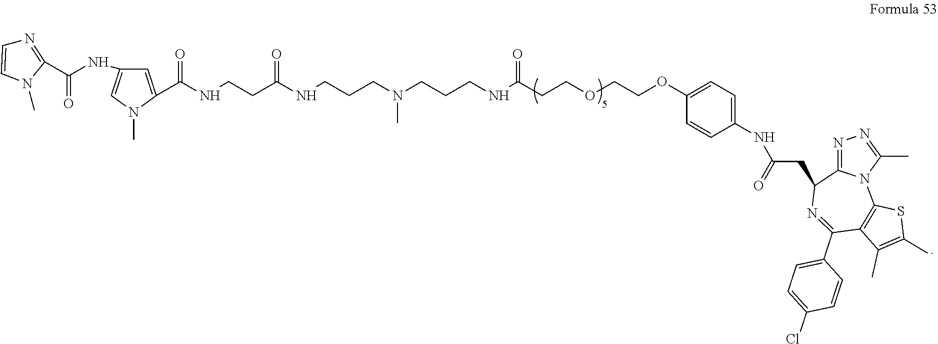

In some embodiments, the synthetic transcription factor has the structure of formula 54:

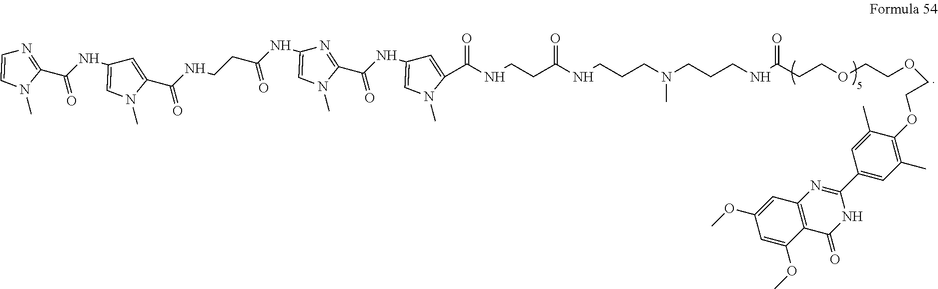

In some embodiments, the synthetic transcription factor has the structure of formula 55:

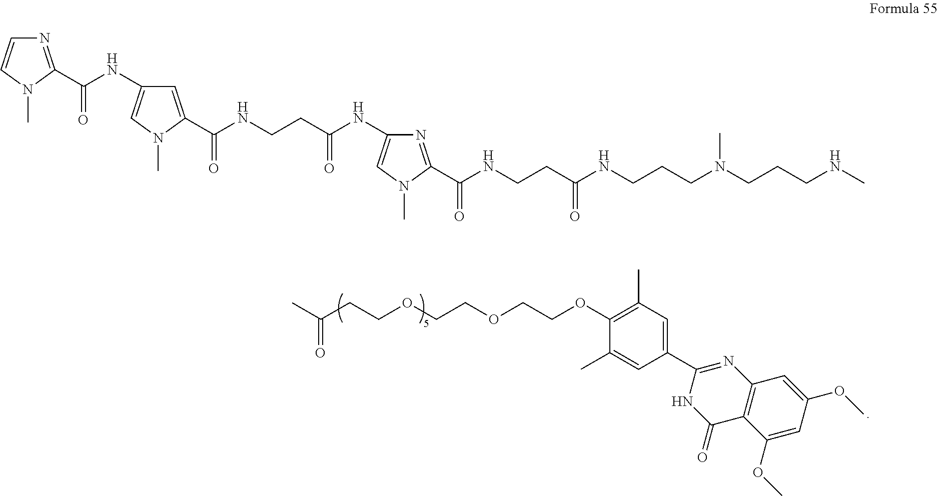

In some embodiments, the synthetic transcription factor has the structure of formula 56:

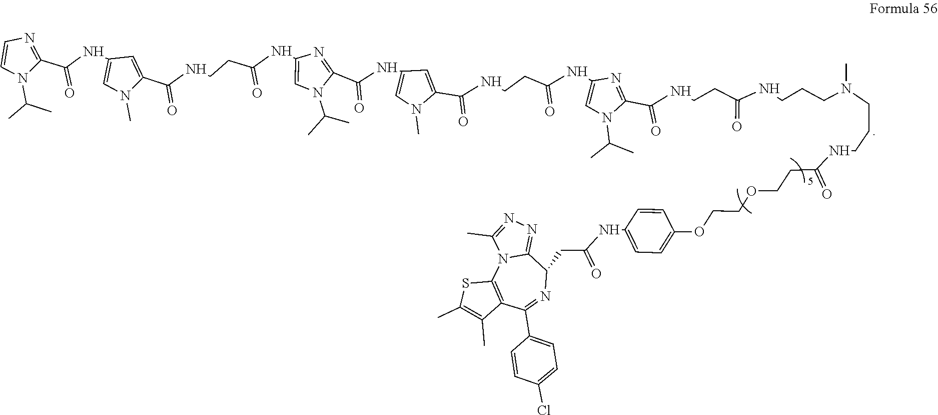

In a second aspect provided herein is a synthetic transcription factor having formula A-L-B wherein -L- is a linker; —B is a nucleic acid binding moiety that specifically binds to one or more repeats of a GAA oligonucleotide sequence; and A- is a bromodomain binding moiety selected from the group consisting of I-BET762, I-BET726, BET-BAY002, CPI 0610, a bromodomain binding moiety having the structure of formula 7:

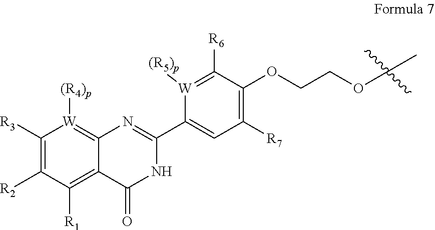

wherein R₁ and R₃ are each independently selected from alkoxy, alkyl, amino, halogen, and hydrogen; R₂ is selected from alkoxy, alkyl, alkenyl, alkynyl, amide, amino, halogen, and hydrogen; R₆ and R₇ are each independently selected from alkyl, alkoxy, amino, halogen, and hydrogen; R₅ is hydrogen; each W is independently selected from C and N, wherein if W is N, then p is 0 or 1, and if W is C, then p is 1; for W—(R₄)ₚ, if W is C, p is 1 and R₄ is H, or if W is N, then p is 0; if R₁ is hydrogen, then R₃ is alkoxy; if R₃ is hydrogen, then R₁ is selected from amino and alkoxy; and at least one of R₆ and R₇ is independently selected from alkyl, alkoxy, amino, and halogen, a bromodomain binding moiety having the structure of formula 9:

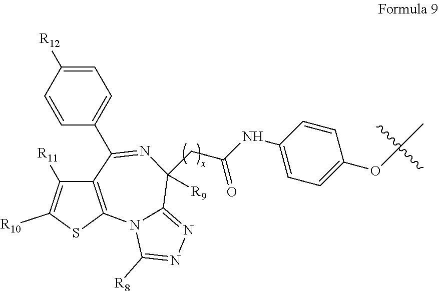

wherein R₈, R₁₀, and R₁₁ are each independently selected from hydrogen, methyl, ethyl, and halomethyl; R₉ is selected from hydrogen, C₁-C₆ alkyl group, and substituted C₁-C₆ alkyl group; R₁₂ is selected from halogen, aryl, substituted aryl, amino, and amido; and X is an integer from 1 to 6, and a bromodomain binding moiety having the structure of formula 20:

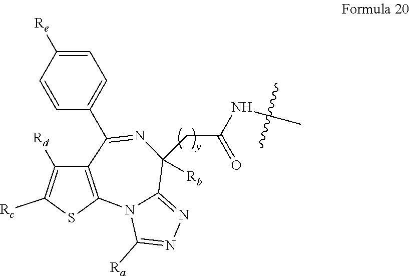

wherein R_{b} is a hydrogen, C₁-C₆ alkyl group, or substituted C₁-C₆ alkyl group; Rₐ, R_{c}, and R_{d} are each independently hydrogen, methyl, ethyl, or halomethyl; Rₑ is a halogen, an aryl, a substituted aryl, amino, or amido group; and y is an integer from 1 to 6; and wherein the synthetic transcription factor has a non-amide bond between A- and -L-.

In some embodiments, the bromodomain binding moiety has the structure of Formula 10. In some embodiments, the bromodomain binding moiety has the structure of Formula 8. In some embodiments, the bromodomain binding moiety has the structure of Formula 22. In some embodiments, —B is a polyamide that specifically binds to one or more repeats of a GAA oligonucleotide. In some embodiments, —B comprises one or more of the following subunits

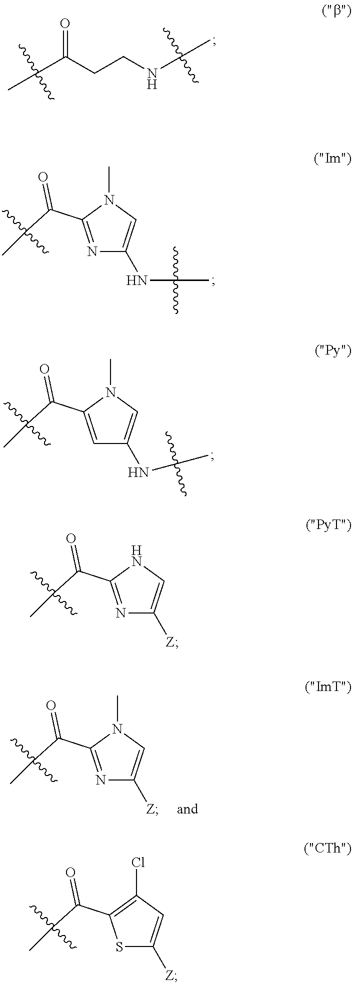

wherein Z is hydrogen, an amino, or an amido group. In some embodiments, —B is selected from the group consisting of PA1, PA5, PA14, PA16, PA17, and PA18, wherein PA1 has the structure of formula 1, PA5 has the structure of formula 2, PA14 has the structure of formula 3, PA16 has the structure of formula 4, PA17 has the structure of formula 5, and PA18 has the structure of formula 6. In some embodiments, the linker is a polyethylene glycol linker. In some embodiments, the synthetic transcription factor has an ether bond between A- and -L-.

In some embodiments, the synthetic transcription factor is selected from the group consisting of formula 35, formula 36, formula 37, formula 38, formula 46, formula 49, formula 52, formula 53, formula 54, formula 55, and formula 56

In a third aspect, provided herein is a synthetic transcription factor having formula C—X-L-B, wherein -L- is a linker; —B is a nucleic acid binding moiety that specifically binds to one or more repeats of a GAA oligonucleotide sequence; C— is a bromodomain binding fragment of a bromodomain binding moiety; and —X— is a substituted or unsubstituted —O—C₆₋₁₀ arylene, a substituted or unsubstituted —O-5-10 membered heteroarylene, or a substituted or unsubstituted —C₆₋₁₀-OM arylene, wherein M is an alkoxy group. In some embodiments, —X— is an unsubstituted —O—C₆₋₁₀ arylene or—O-5-10 membered heteroarylene. In some embodiments, —X— is a —O—C₆₋₁₀ aryl or —O-5-10 membered heteroaryl substituted with one to five substituents selected from the group consisting of halogen, C₁₋₆ alkyl, C₁₋₆ alkoxy, amino, hydroxy, oxo, and carboxylic acid. In some embodiments, —X— is an unsubstituted

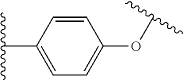 [SMILES: *Oc1ccc(*)cc1]

or a

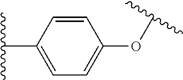 [SMILES: *Oc1ccc(*)cc1]

substituted with one to five substituents selected from the group consisting of halogen, C₁₋₆ alkyl, C₁₋₆ alkoxy, amino, hydroxy, oxo, and carboxylic acid. In some embodiments, C— is a bromodomain binding fragment of a bromodomain inhibitor. In some embodiments, C—X— has the structure of formula I:

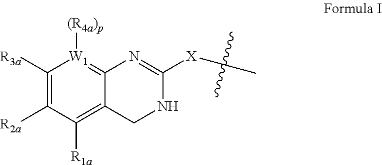

wherein R₁ₐ and R₃ₐ are each independently selected from alkoxy, alkyl, amino, halogen, and hydrogen; R₂ₐ is selected from alkoxy, alkyl, alkenyl, alkynyl, amide, amino, halogen, and hydrogen; W₁ is independently selected from C and N, wherein if W₁ is C, p is 1 and R₄ₐ is H or if W is N, p is 0. In some embodiments, C—X— has the structure of formula I-1:

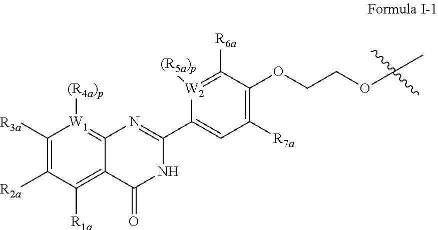

wherein R₆ₐ and R₇ₐ are each independently selected from alkyl, alkoxy, amino, halogen, and hydrogen; R₅ₐ is hydrogen; W₂ is independently selected from C and N, wherein when is W₂ is N, p is 0 or 1 and when W₂ is C, then p is 1. In some embodiments, C—X has the structure of formula 8:

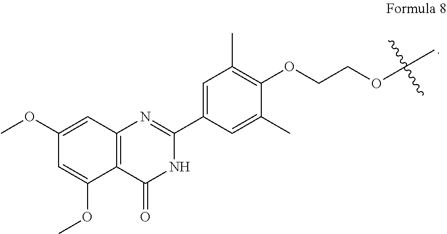 [SMILES: *OCCOc1c(C)cc(-c2nc3cc(OC)cc(OC)c3c(=O)[nH]2)cc1C]

In some embodiments, C—X has the structure of formula 9:

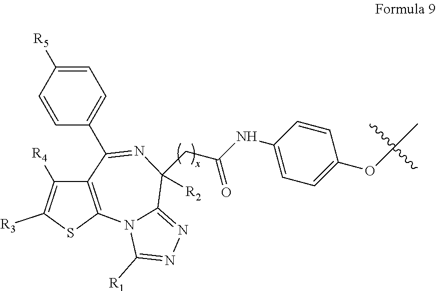

wherein R₁, R₃, and R₄ are each independently selected from hydrogen, methyl, ethyl, and halomethyl; R₂ is selected from hydrogen, C₁-C₆ alkyl group, and substituted C₁-C₆ alkyl group; R₅ is selected from halogen, aryl, substituted aryl, amino, and amido; and x is an integer from 1 to 6. In some embodiments, C—X has the structure of formula 10:

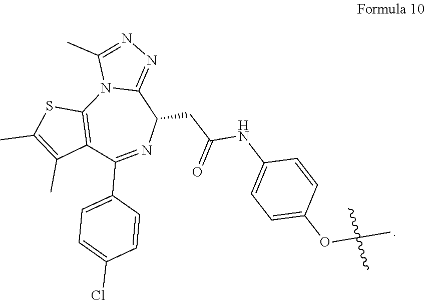 [SMILES: *Oc1ccc(NC(=O)C[C@@H]2N=C(c3ccc(Cl)cc3)c3c(sc(C)c3C)-n3c(C)nnc32)cc1]

In some embodiments, —B is a polyamide that specifically binds to one or more repeats of a GAA oligonucleotide. In some embodiments, —B comprises one or more of the following

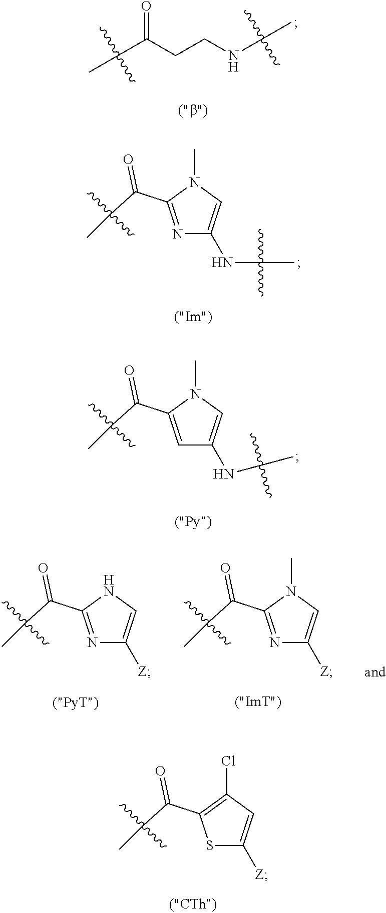

subunits:

wherein Z is hydrogen, an amino, or an amido group. In some embodiments, —B is selected from the group consisting of PA1, PA5, PA14, PA16, PA17, and PA18, wherein PA1 has the structure of formula 1, PA5 has the structure of formula 2, PA14 has the structure of formula 3, PA16 has the structure of formula 4, PA17 has the structure of formula 5, and PA18 has the structure of formula 6. In some embodiments, the linker is a polyethylene glycol linker. In some embodiments, the synthetic transcription factor has a non-amide bond between —X— and -L-. In some embodiments, the synthetic transcription factor has an ether bond between —X— and -L-. In some embodiments, the synthetic transcription factor has a non-amide bond between -L- and —B. In some embodiments, the linker comprises a linking moiety selected from the group consisting of—O—, —(CH₂)ₓ—, —(CH₂CH₂O)_{y}—, —(OCH₂CH₂)_{y}—, —C(O)NR′—, —NR′C(O)—, —C(O)—, —NR*—, and

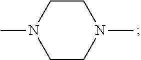

wherein R′ and R* are each independently a hydrogen or C₁-C₆ alkyl; and x and y are each independently an integer from 1 to 10. In some embodiments, R′ is hydrogen and R* is —CH₃.

In some embodiments, the synthetic transcription factor has the structure of Formula 35, Formula 36, Formula 37, Formula 38, Formula 46, Formula 49, Formula 51, Formula 52, Formula 53, Formula 54, Formula 55, or Formula 56.

In a forth aspect, provided herein is a method for increasing frataxin (FXN) levels in a cell comprising contacting the cell with an effective amount of a synthetic transcription factor described herein. In some embodiments, the cell comprises a frataxin (FXN) gene including at least about 30 GAA repeats.

In a fifth aspect, provided herein is a pharmaceutical composition comprising a synthetic transcription factor described herein and a pharmaceutically acceptable carrier.

In a sixth aspect, provided herein is a method for treating Friedreich's ataxia in a subject in need thereof, comprising administering to the subject a therapeutically effective amount of a synthetic transcription factor or a pharmaceutical composition described herein. In some embodiments, frataxin (FXN) mRNA levels are increased relative to those in the subject prior to treatment. In some embodiments, frataxin (FXN) protein levels are increased relative to those in the subject prior to treatment. In some embodiments, the treatment comprises ameliorating one or more symptoms of Friedreich's ataxia (FRDA).

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF DRAWINGS

The patent or patent application file contains at least one drawing in color. Copies of this patent or patent application publication with color drawings will be provided by the Office upon request and payment of the necessary fee.

FIG. 1 shows the structure and mass spectra of SynTEF1.

FIG. 2 shows the mass spectra of SynTEF1 after 5 days at 18° C. and 40 days at —20° C.

FIG. 3 shows the location of cleavage susceptible amide bonds in SynTEF1.

FIG. 4 shows the structure and mass spectra of SynTEF3 (PA1-PEG₅-OTX).

FIG. 5 shows the structure and mass spectra of SynTEF5 (PA1-PEG₅-RVX-NEW).

FIG. 6 shows the structure of SynTEF11 (PA5-PEG₅-OTX).

FIG. 7 shows the mass spectra of SynTEF11 (PA5-PEG₅-OTX).

FIG. 8 shows FXN expression in GM04078 fibroblasts with treated with DMSO (control), PA1, JQ1, SynTEF1, PA1-RVX (old, amide bond containing), SynTEF5 (PA1-RVX new, ether bond containing), or SynTEF3 (PA1-PEG₅-OTX). Cells were treated with SynTEF3 (PA1-PEG₅-OTX) that was freshly synthesized (new) or that had been synthesized one month prior and stored in DMSO at −80° C. (old).

FIG. 9 shows FXN expression in GM04078 fibroblasts. Expression of FXN mRNA in GM04078 cell line by quantitative RT-PCR. Results are mean±SEM (n=3). All treatments are 24 h with 1 μM of the indicated molecule, except DMSO (0.1%).

FIG. 10 shows the structure of SynTEF4 in which the amide bond connecting the nucleic acid binding moiety and the linker has been replaced with a secondary amine.

FIG. 11 shows FXN expression in GM04078 and GM15850 cells treated with DMSO, SynTEF1, or SynTEF4 for 24, 48, or 72 hours. The GM04078 cell line was also treated with SynTEF3.

FIG. 12 shows expression of FXN in GM15850 lymphoblastoid cells. GM 15850 cells were treated with DMSO, SynTEF1, or PA1-PEG₅-RVX including an amide bond between the linker domain and the bromodomain binding moiety.

FIGS. 13A-13B shows expression of FXN in GM04078 fibroblast cells. FIG. 13A shows FXN expression following 24 h treatment with DMSO, SynTEF1, 10 μM SynTEF3, 1 μM SynTEF3, 10 μM SynTEF11, or 1 μM SynTEF11. FIG. 13B shows FXN expression following 24 h treatment with DMSO, SynTEF4, SynTEF3, or SynTEF1. In FIG. 13B, molecules were tested at a final concentration of 1 μM.

FIG. 14 shows the synthesis of SynTEF3 (PA1-PEG₅-OTX).

FIG. 15 show the mass spectra of PA1 following the synthesis outlined in Scheme 1 of FIG. 14.

FIG. 16 shows the mass spectra of OTX-PEG₅-COOH.

FIG. 17 shows the mass spectra of SynTEF3.

FIG. 18 shows the synthesis of SynTEF5 (PA1-PEG₅-RVX new).

FIG. 19 shows the mass spectra of SynTEF5.

FIG. 20 shows the synthesis of SynTEF11 (PA5-PEG₅-OTX).

FIG. 21 shows the mass spectra of PA5 and SynTEF11.

FIG. 22 shows the synthesis of SynTEF4.

FIG. 23 shows the mass spectra of OTX-PEG₅-Br (top) and the mass spectra of SynTEF4 (bottom).

FIG. 24 shows the structure and mass spectra of PA14.

FIG. 25 shows the structure and mass spectra of PA16.

FIG. 26 shows the structure and mass spectra of PA17.

FIG. 27 shows the structure and mass spectra of PA18.

FIG. 28 shows the structure and mass spectra of (+)-JQ1-PEG₈-COOH.

FIG. 29 shows the structure and mass spectra of (+)-JQ1-PEG₁₀-COOH.

FIG. 30 shows the structure and mass spectra of I-BET762-PEG₆-COOH.

FIG. 31 shows the structure and mass spectra of I-BET762-PEG₈-COOH.

FIG. 32 shows the structure and mass spectra of SynTEF33.

FIG. 33 shows the structure and mass spectra of SynTEF34.

FIG. 34 shows the structure and mass spectra of SynTEF35.

FIG. 35 shows the structure and mass spectra of SynTEF36.

FIG. 36 shows the structure and mass spectra of SynTEF37.

FIG. 37 shows the structure and mass spectra of SynTEF38.

FIG. 38 shows the structure and mass spectra of SynTEF39.

FIG. 39 shows the structure and mass spectra of SynTEF40.

FIG. 40 shows the structure of SynTEF41.

FIG. 41 shows the structure and mass spectra of SynTEF42.

FIG. 42 shows the structure and mass spectra of SynTEF43.

FIG. 43 shows the structure and mass spectra of SynTEF44.

FIG. 44 shows the synthesis of Formula 30, wherein A- is RVX and -L- is a PEG linker.

FIG. 45 shows SynTEF3 stability at day 0 at 37° C.

FIG. 46 shows SynTEF3 stability at day 1 at 37° C.

FIG. 47 shows SynTEF3 stability at day 14 at 37° C.

### DETAILED DESCRIPTION OF THE INVENTION

The present disclosure is based on the design of synthetic transcription factors for enhancing expression of frataxin (FXN). The synthetic transcription factors generally include a bromodomain binding moiety, a linker, and a nucleic acid binding moiety. Without wishing to be bound by any particular theory, conjugation of the bromodomain binding ligands to DNA binding polyamides generates molecules that can non-covalently recruit Brd4 and other bromodomain containing proteins to genomic loci that are bound by the designated polyamide module. In cells or in vivo, these molecules traffic to the nucleus and associate with the genomic loci designated by the polyamide module. At the designed site, the molecules increase the local concentration of the bromodomain containing proteins in a manner that reflects the affinities and stabilities of the bromodomain binding ligand in the complex cellular milieu.

### Compositions

The present technology discloses a synthetic transcription factor of the formula A-L-B, wherein -L- is a linker; A- is a bromodomain binding moiety; and —B is a nucleic acid binding moiety. The synthetic transcription factors are specific to GAA repeat sequences and have increased stability and activity compared to other GAA repeat specific synthetic transcription factors previous described. See for example, US Patent Publication No. 2017/0281643, which is incorporated herein in its entirety.

In some embodiments, the nucleic acid binding moiety (—B) specifically binds to a target oligonucleotide sequence. In some embodiments, the nucleic acid binding moiety (—B) specifically binds to one or more repeats of a short oligonucleotide sequence such as a GAA oligonucleotide sequence. In some embodiments, the nucleic acid binding moiety (—B) is a polyamide. In some embodiments, the nucleic acid binding moiety (—B) is a polyamide that specifically binds to one or more repeats of an oligonucleotide sequence containing 3 to 6 nucleotides, such as a GAA oligonucleotide sequence. In some embodiments, the nucleic acid binding moiety (—B) comprises an oligonucleotide sequence (e.g., containing about 15 to 30 nucleotides) that is complementary to a desired target oligonucleotide sequence. In some embodiments, the nucleic acid binding moiety (—B) may be a nucleic acid sequence capable of hybridizing to one or more repeats of a GAA oligonucleotide sequence or to one or more repeats of a TTC oligonucleotide sequence. In some embodiments, the nucleic acid binding moiety (—B) may be a deoxyribonucleic acid (DNA) sequence, a ribonucleic acid (RNA) sequence, or a peptide nucleic acid (PNA) sequence capable of hybridizing to one or more repeats of a GAA oligonucleotide sequence or to one or more repeats of a TTC oligonucleotide sequence. For example, the nucleic acid binding moiety (—B) may be a deoxyribonucleic acid (DNA) sequence comprising, consisting of, or consisting essentially of one or more repeats of a TTC sequence, including, but not limited to, TTCTTCTTC, TTCTTCTTCTTC(SEQ ID NO:1), TTCTTCTTCTTCTTC(SEQ ID NO:2), TTCTTCTTCTTCTTCTTC(SEQ ID NO:3), and TTCTTCTTCTTCTTCTTCTTC(SEQ ID NO:4). In another example, the nucleic acid binding moiety (—B) may be a deoxyribonucleic acid (DNA) sequence comprising, consisting of, or consisting essentially of one or more repeats of a GAA sequence, including, but not limited to, GAAGAAGAA, GAAGAAGAAGAA (SEQ ID NO:5), GAAGAAGAAGAAGAA (SEQ ID NO: 6), GAAGAAGAAGAAGAAGAA (SEQ ID NO:7), and GAAGAAGAAGAAGAAGAAGAA (SEQ ID NO:8). In another example, the nucleic acid binding moiety (—B) may be a ribonucleic acid (RNA) sequence comprising, consisting of, or consisting essentially of one or more repeats of a CUU sequence, including, but not limited to, CUUCUUCUU, CUUCUUCUUCUU (SEQ ID NO:9), CUUCUUCUUCUUCUU (SEQ ID NO: 10), CUUCUUCUUCUUCUUCUU (SEQ ID NO:11), and CUUCUUCUUCUUCUUCUUCUU (SEQ ID NO:12).

In some embodiments, the nucleic acid binding moiety (—B) may be a deoxyribonucleic acid (DNA) sequence comprising, consisting of, or consisting essentially of 1 to 10, 1 to 15, 1 to 20, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, 1 to 100, 1 to 150, 1 to 200, 1 to 250, 1 to 300, 1 to 350, 1 to 400, 1 to 450, 1 to 500, 1 to 550, 1 to 600, 1 to 650, 1 to 700, 1 to 750, 1 to 800, 1 to 850, 1 to 900, 1 to 950, or 1 to 1000 repeats of a TTC sequence. In some embodiments, the nucleic acid binding moiety (—B) may be a DNA sequence of 5 to 10 repeats of a TTC sequence (e.g., 15 to 30 nucleotide bases in length). In some embodiments, the nucleic acid binding moiety (—B) may be a DNA sequence of 5 to 6 repeats of a TTC sequence. In some embodiments, the nucleic acid binding moiety (—B) may be a DNA sequence of 5 to 7 repeats of a TTC sequence. In some embodiments, the nucleic acid binding moiety (—B) may be a DNA sequence of 5 to 8 repeats of a TTC sequence. In some embodiments, the nucleic acid binding moiety (—B) may be a DNA sequence of 5 to 9 repeats of a TTC sequence.

In some embodiments, the nucleic acid binding moiety (—B) may be a deoxyribonucleic acid (DNA) sequence comprising, consisting of, or consisting essentially of 1 to 10, 1 to 15, 1 to 20, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, 1 to 100, 1 to 150, 1 to 200, 1 to 250, 1 to 300, 1 to 350, 1 to 400, 1 to 450, 1 to 500, 1 to 550, 1 to 600, 1 to 650, 1 to 700, 1 to 750, 1 to 800, 1 to 850, 1 to 900, 1 to 950, or 1 to 1000 repeats of a GAA sequence. In some embodiments, the nucleic acid binding moiety (—B) may be a DNA sequence of 5 to 10 repeats of a GAA sequence (e.g., 15 to 30 nucleotide bases in length). In some embodiments, the nucleic acid binding moiety (—B) may be a DNA sequence of 5 to 6 repeats of a GAA sequence. In some embodiments, the nucleic acid binding moiety (—B) may be a DNA sequence of 5 to 7 repeats of a GAA sequence. In some embodiments, the nucleic acid binding moiety (—B) may be a DNA sequence of 5 to 8 repeats of a GAA sequence. In some embodiments, the nucleic acid binding moiety (—B) may be a DNA sequence of 5 to 9 repeats of a GAA sequence.

In some embodiments, the nucleic acid binding moiety (—B) may be a ribonucleic acid (RNA) sequence comprising, consisting of, or consisting essentially of 1 to 10, 1 to 15, 1 to 20, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, 1 to 100, 1 to 150, 1 to 200, 1 to 250, 1 to 300, 1 to 350, 1 to 400, 1 to 450, 1 to 500, 1 to 550, 1 to 600, 1 to 650, 1 to 700, 1 to 750, 1 to 800, 1 to 850, 1 to 900, 1 to 950, or 1 to 1000 repeats of a CUU sequence. In some embodiments, the nucleic acid binding moiety (—B) may be an RNA sequence of 5 to 10 repeats of a CUU sequence (e.g., 15 to 30 nucleotide bases in length). In some embodiments, the nucleic acid binding moiety (—B) may be an RNA sequence of 5 to 6 repeats of a CUU sequence. In some embodiments, the nucleic acid binding moiety (—B) may be an RNA sequence of 5 to 7 repeats of a CUU sequence. In some embodiments, the nucleic acid binding moiety (—B) may be an RNA sequence of 5 to 8 repeats of a CUU sequence. In some embodiments, the nucleic acid binding moiety (—B) may be an RNA sequence of 5 to 9 repeats of a CUU sequence.

In some embodiments, the nucleic acid binding moiety (—B) comprises a repeat-targeted duplex RNA, such as an anti-GAA duplex RNA that specifically targets GAA repeats. In some embodiments, the nucleic acid binding moiety (—B) comprises single-stranded locked nucleic acids (LNAs), such as anti-GAA LNA oligomers that specifically target GAA repeats.

In some embodiments, —B may be a polyamide and comprise one or more of the following subunits:

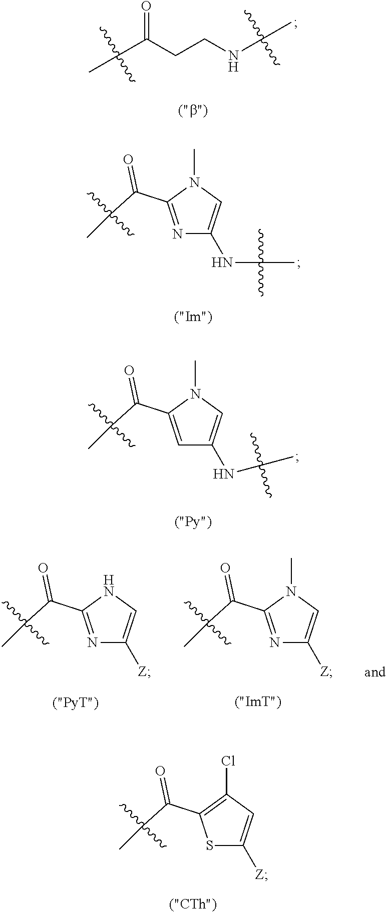

in which Z is typically hydrogen, amino, or an amido group.

In some embodiments, the one or more repeats may be GAA. In some embodiments, —B may specifically bind to a one or more repeats of a GAA oligonucleotide sequence. In some embodiments, —B may include —X—(β-Py-Im)ₙ-β-Py-TRM; where X is -β-Im-, -β-Py-, -β-, or a bond; n is 1-10; and -TRM is -ImT or -CTh; with the proviso that one of the -β-Py-Im-trimers may be replaced by a -β-Im-Im-trimer. In some embodiments, —B may include —X—(β-Py-Im)ₙ—(β-Py-ImT); wherein: X is -β-Im-, -β-Py-, -β-, or a bond; Z is hydrogen, amino, or amido group; and n is 0 to 10; with the proviso that when n is at least 1, one of the -β-Py-Im-trimers may be replaced by a -β-Im-Im-trimer.

In some embodiments, Z may be —NR^{B}R^{B} or —N⁺R^{A}R^{B}R^{B}; wherein R^{A} may be hydrogen; and R^{B} may be a hydrogen, C₁-C₆ alkyl, C₁-C₆ alkenyl, or C₁-C₆ alkynyl group. In some embodiments, Z may be —N(R^{A})C(O)R^{B}; wherein R^{A} may be hydrogen; and R^{B} may be a hydrogen, C₁-C₆ alkyl, C₁-C₆ alkenyl, or C₁-C₆ alkynyl group. In some embodiments, R^{B} may be hydrogen or C₁-C₆ alkyl group. In some embodiments, R^{B} may be hydrogen or —CH₃. In certain embodiments, Z may be —NH₂. In certain embodiments, Z may be —NH₃+. In certain embodiments, Z may be hydrogen.

In some embodiments, n may be an integer from 1 to 10. In some embodiments, n may be 1, 2, 3, 4, or 5. In certain embodiments, n may be 1 or 2. In some embodiments, n may be 1 or 2 and none of the -β-Py-Im-trimers are replaced by a -β-Im-Im-trimer. In some embodiments, n may be 1 or 2 and one of the -β-Py-Im-trimers is replaced by a -β-Im-Im-trimer.

In some embodiments, —B may be -(β-Py-Im)ₙ-(β-Py-ImT); wherein Z may be hydrogen or NH₃⁺ and n may be 1 or 2.

In some embodiments, —B may include -β-Im-β-Py-Im-β-Py-ImT, -β-β-Py-Im-β-Py-ImT, -β-Py-Im-β-Py-ImT, -β-Im-β-Py-Im-β-Py-Im-β-Py-ImT, -β-Py-Im-β-Py-Im-β-Py-ImT, and/or -β-β-Py-Im-β-Py-Im-β-Py-ImT; in which Z may be hydrogen. In some embodiments, —B may include-β-Im-β-Py-Im-β-Py-Im-β-Py-ImT, -β-Im-β-Py-Im-β-Im-Im-β-Py-ImT, and/or -β-Im-β-Im-Im-β-Py-Im-β-Py-ImT; in which Z may be hydrogen. In some embodiments, —B may include -β-Py-β-Py-Im-β-Py-ImT, -β-β-Py-Im-β-Py-ImT, -β-Im-β-Py-Im-β-Py-Im-β-Py-ImT, -β-Py-β-Py-Im-β-Py-Im-β-Py-ImT, -β-Py-Im-β-Py-Im-β-Py-ImT, -β-Py-β-Py-Im-β-Py-CTh and/or -β-β-Py-Im-β-Py-Im-β-Py-ImT, -β-Py-Im-β-Py-CTh.

In some embodiments, —B is the polyamide PA1 having the structure of formula 1:

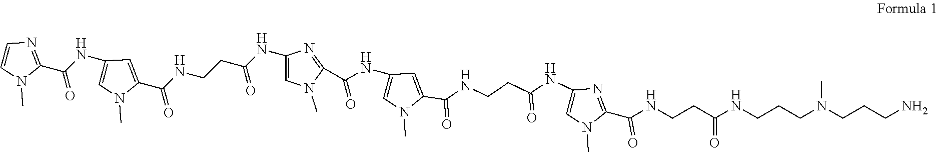 [SMILES: CN(CCCN)CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C]

In some embodiments, —B is the polyamide PA5 having the structure of formula 2:

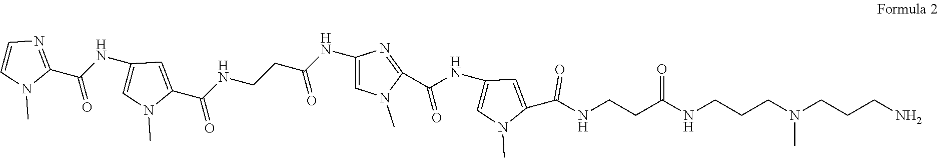 [SMILES: CN(CCCN)CCCNC(=O)CCNC(=O)c1cc(NC(=O)c2nc(NC(=O)CCNC(=O)c3cc(NC(=O)c4nccn4C)cn3C)cn2C)cn1C]

In some embodiments, —B is the polyamide PA14 having the structure of formula 3:

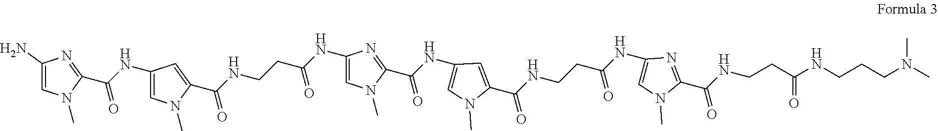 [SMILES: CN(C)CCCNC(=O)CCNC(=O)c1nc(NC(=O)CCNC(=O)c2cc(NC(=O)c3nc(NC(=O)CCNC(=O)c4cc(NC(=O)c5nc(N)cn5C)cn4C)cn3C)cn2C)cn1C]

In some embodiments, —B is the polyamide PA16 having the structure of formula 4:

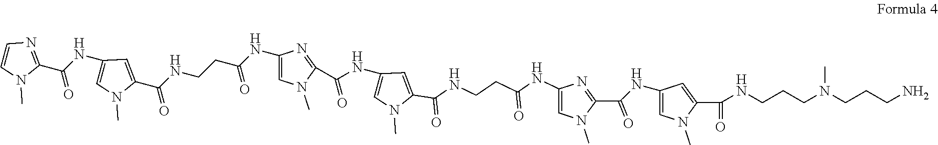 [SMILES: CN(CCCN)CCCNC(=O)c1cc(NC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)cn1C]

In some embodiments, —B is the polyamide PA17 having the structure of formula 5:

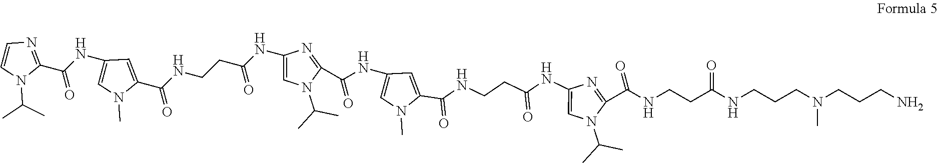 [SMILES: CC(C)n1cc(NC(=O)CCNC(=O)c2cc(NC(=O)c3nc(NC(=O)CCNC(=O)c4cc(NC(=O)c5nccn5C(C)C)cn4C)cn3C(C)C)cn2C)nc1C(=O)NCCC(=O)NCCCN(C)CCCN]

In some embodiments, —B is the polyamide PA18 having the structure of formula 6:

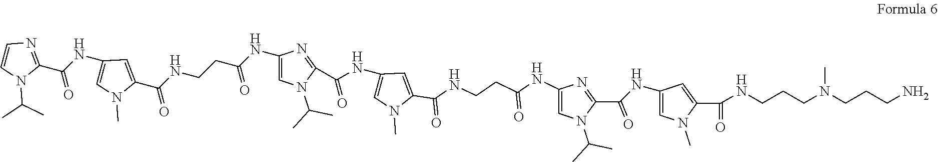 [SMILES: CC(C)n1ccnc1C(=O)Nc1cc(C(=O)NCCC(=O)Nc2cn(C(C)C)c(C(=O)Nc3cc(C(=O)NCCC(=O)Nc4cn(C(C)C)c(C(=O)Nc5cc(C(=O)NCCCN(C)CCCN)n(C)c5)n4)n(C)c3)n2)n(C)c1]

TABLE 1
Summary of exemplary polyamides

| Polyamide Name | Molecular Formula | Molecular Weight |
|---|---|---|
| PA1 | C₄₃H₆₀N₁₈O₈ | 956.48 |
| PA5 | C₃₅H₅₀N₁₄O₆ | 762.40 |
| PA14 | C₄₁H₅₆N₁₈O₈ | 928.45 |
| PA16 | C₄₆H₆₁N₁₉O₈ | 1007.50 |
| PA17 | C₄₉H₇₂N₁₈O₈ | 1040.58 |
| PA18 | C₅₂H₇₃N₁₉O₈ | 1091.59 |

The A- subunit is a bromodomain binding moiety. As used herein, “bromodomain binding moiety” refers to a compound that binds to a bromodomain, also known in the art as an acetyl-lysine recognition motif. Despite sequence variability, all bromodomains share a common fold that includes a left-handed bundle of four α-helices (α_{Z}, α_{A}, α_{B}, α_{C}), linked by loop regions (ZA loop and BC loop) that contribute to substrate specificity. Acetyl-lysine is recognized by a central hydrophobic cavity and is anchored by a hydrogen bond with an asparagine residue present in most bromodomains. Bromodomain-containing proteins are known in the art. For example, bromodomain containing proteins include, but are not limited to, CECR2, FALZ, GCN5L2, PCAF, BAZIB, BRD8B, BRWD3, CREBBP, EP300, PHIP, WDR9(2), ATAD2, BRD1, BRD7, BRD9, BRFPF1, BRFPF3, KIAA1240, BAZ2A, BAZ2B, LOC93349, SP100, SP110, SP140, TIF1α, TRIM33, TRIM66, MLL, TRIM28, PRKCBP1, TAF1, TAF1L, WDR9(6), ZMYND11, PB1, SMARCA2, SMARCA4, bromodomain-containing protein 2 (BRD2), bromodomain-containing protein 3 (BRD3), bromodomain-containing protein 4 (BRD4), and bromodomain testis-specific protein (BRDT). Additional bromodomain containing proteins are known in the art. See for example Liu et al. (“Drug discovery targeting bromodomain-containing protein 4,” J. Med. Chem., 2017, 60, 4533-4558). In some embodiments, the A- subunit is a Brd4 binding ligand.

In some embodiments, the bromodomain containing protein is a member of the bromodomain and extra-terminal (BET) family, which includes BRD2, BRD3, BRD4, and BRDT. The BET family shares a common domain architecture including two amino-terminal bromodomains and a more divergent carboxy-terminal recruitment domain.

Bromodomain binding moieties are known in the art. Suitable bromodomain binding moieties include, but are not limited to, (+)-JQ1, OTX015, I-BET762, RVX208, I-BET726, BET-BAY002, and CPI0610. See, for example Nicodeme et al. (“Suppression of inflammation by a synthetic histone mimic,” Nature, 2010, 468:1119-1123), Liu et al. (“Drug discovery targeting bromodomain-containing protein 4,” J. Med. Chem., 2017, 60, 4533-4558), and Filippakopoulos et al. (“Selective inhibition of BET bromodomains,” Nature, 2010, 468:1067-1073) for examples of bromodomain binding moieties known in the art. In some embodiments, the bromodomain binding moieties is selected from those outlined in Table 2. In some embodiments, the bromodomain binding moiety is selected from the group consisting of (+)-JQ1, OTX015, I-BET762, RVX208, I-BET726, BET-BAY002, and CPI0610.

In some embodiments, the bromodomain binding moiety is a BET family binding moiety. As used herein “BET family binding moiety” refers to a compound that binds to the bromodomain of a BET family member, such as BRD2, BRD3, BRD4, or BRDT.

TABLE 2
BET family binding moieties

| Structure | Name | Description |
|---|---|---|
| 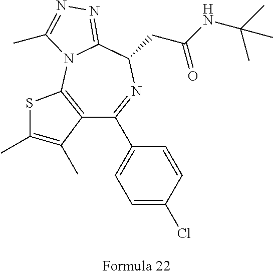 [SMILES: Cc1sc2c(c1C)C(c1ccc(Cl)cc1)=N[C@@H](CC(=O)NC(C)(C)C)c1nnc(C)n1-2] | (+)-JQ1 | (+)-JQ-1 is a BET bromodomain inhibitor, with IC50s of 77 and 33 nM for the first and second bromodomain (BRD4(1/2)). (+)-JQ-1 also activates autophagy. |
| 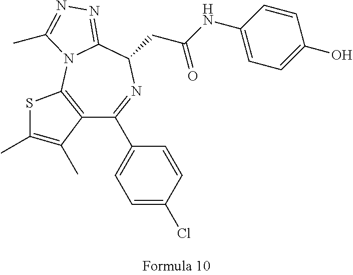 [SMILES: Cc1sc2c(c1C)C(c1ccc(Cl)cc1)=N[C@@H](CC(=O)Nc1ccc(O)cc1)c1nnc(C)n1-2] | OTX015 | Birabresib (OTX-015) is a potent bromodomain (BRD2/3/4) inhibitor with IC50s ranging from 92 to 112 nM. |
| 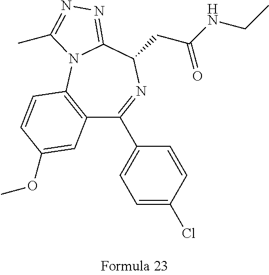 [SMILES: CCNC(=O)C[C@@H]1N=C(c2ccc(Cl)cc2)c2cc(OC)ccc2-n2c(C)nnc21] | I-BET762 | Molibresib (GSK 525762A; I-BET 762) is a BET bromodomain inhibitor with IC50 of 32.5-42.5 nM. |
| 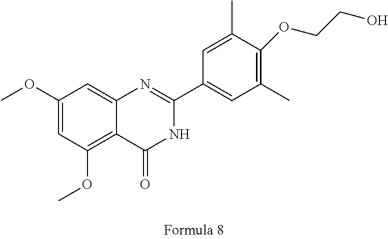 [SMILES: COc1cc(OC)c2c(=O)[nH]c(-c3cc(C)c(OCCO)c(C)c3)nc2c1] | RVX208 | Apabetalone (RVX-208) is an inhibitor of BET transcriptional regulators with selectivity for the second bromodomain. The IC50s are 87 ± 10 μM and 0.51 ± 0.041 μM for BD1 and BD2, respectively. |
| 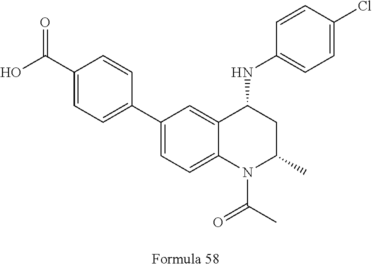 [SMILES: CC(=O)N1c2ccc(-c3ccc(C(=O)O)cc3)cc2[C@H](Nc2ccc(Cl)cc2)C[C@@H]1C] | I-BET726 | GSK 1324726A is a novel, potent, and selective inhibitor of BET proteins with high affinity to BRD2 (IC50 = 41 nM), BRD3 (IC50 = 31 nM), and BRD4 (IC50 = 22 nM). |
| 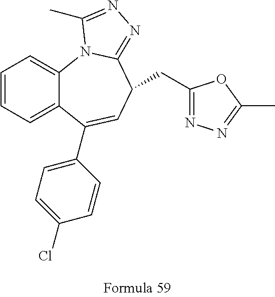 [SMILES: Cc1nnc(C[C@@H]2C=C(c3ccc(Cl)cc3)c3ccccc3-n3c(C)nnc32)o1] | BET- BAY002 | BET-BAY 002 S- enantiomer is the S- enantiomer of BET- BAY 002. BET-BAY 002 is a BET inhibitor. |
| 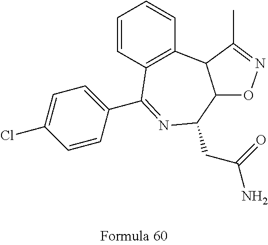 [SMILES: CC1=NOC2C1c1ccccc1C(c1ccc(Cl)cc1)=N[C@H]2CC(N)=O] | CPI 0610 | CPI-0610 is a potent, selective, and cell-active BET bromodomain inhibitor CPI-0610 inhibits BRD4-BD1 with IC50 of 39 nM in time resolved fluorescence energy transfer (TRFRET) binding assay. |

In some embodiments, A- is a bromodomain binding moiety having the structure of formula 7:

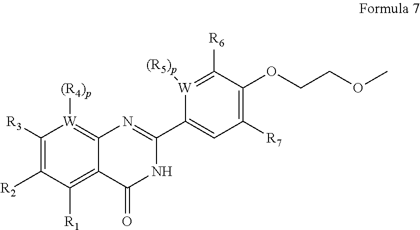

wherein:
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₆ and R₇ are each independently selected from alkyl, alkoxy, amino, halogen, and hydrogen;
R₅ is hydrogen;
each W is independently selected from C and N, wherein if W is N, then p is 0 or 1, and if W is C, then p is 1;
for W—(R₄)ₚ, W is C, p is 1 and R₄ is H, or W is N and p is 0;
with the proviso that if R₁ is hydrogen, then R₃ is alkoxy;
with the proviso that if R₃ is hydrogen, then R₁ is selected from amino and alkoxy; and
at least one of R₆ and R₇ is independently selected from alkyl, alkoxy, amino, and halogen.

In some embodiments, A- is RVX and has the structure of formula 8:

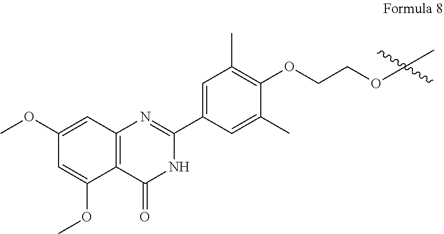 [SMILES: *OCCOc1c(C)cc(-c2nc3cc(OC)cc(OC)c3c(=O)[nH]2)cc1C]

In some embodiments, A- is a bromodomain binding moiety having the structure of formula 9:

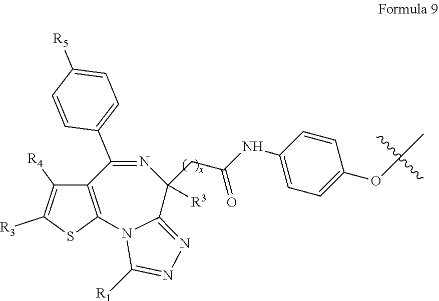

wherein:
R₁, R₃, and R₄ are each independently selected from hydrogen, methyl, ethyl, and halomethyl (e.g., trifluoromethyl);
R₂ is selected from hydrogen, C₁-C₆ alkyl group, and substituted C₁-C₆ alkyl group;
R₅ is selected from halogen, aryl, substituted aryl, amino, and amido; and
X is an integer from 1 to 6 (i.e., 1, 2, 3, 4, 5, or 6).

In some embodiments, A-is OTX and has the structure of formula 10:

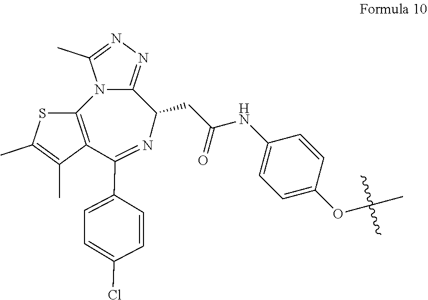 [SMILES: *Oc1ccc(NC(=O)C[C@@H]2N=C(c3ccc(Cl)cc3)c3c(sc(C)c3C)-n3c(C)nnc32)cc1]

In some embodiments, A- is a triazolodiazepine Brd4 binding moiety. In some embodiments A- is a triazolodiazepine Brd4 binding moiety of formula 11:

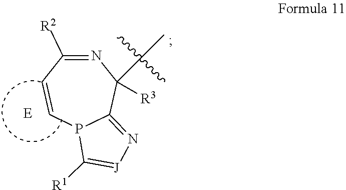

wherein J is N, O or CR¹¹; K is N, O or CR¹¹; with the proviso that J and K cannot both be —O—; P is N, except when one of J or K is O, then P is C; R¹ may be a hydrogen or optionally substituted alkyl, hydroxyalkyl, aminoalkyl, alkoxyalkyl, halogenated alkyl, hydroxyl, alkoxy, or —COOR⁴; wherein R⁴ may be a hydrogen, optionally substituted aryl, aralkyl, cycloalkyl, heteroaryl, heteroaralkyl, heterocycloalkyl, alkyl, alkenyl, alkynyl, or cycloalkylalkyl group optionally interrupted by one or more heteroatoms; R² may be an optionally substituted aryl, alkyl, cycloalkyl, or aralkyl group; R³ may be a hydrogen, halogen, or optionally substituted alkyl group (e.g., —(CH₂)_{b}—C(O)N(R²⁰)(R²¹), —(CH₂)_{b}—N(R²⁰)C(O)(R²¹), or halogenated alkyl group, wherein b may be an integer from 1 to 10, and R²⁰ and R²¹ may independently be a hydrogen or C₁-C₆ alkyl group (typically R²⁰ may be a hydrogen and R²¹ may be a methyl)); R¹¹ may be a hydrogen or optionally substituted alkyl, cycloalkyl, heterocycloalkyl, aryl, or heteroaryl group; and Ring E may be an optionally substituted aryl or heteroaryl ring. In some embodiments, J may be N or CR¹¹. In some embodiments, P is N and J may be CR¹¹, where R¹¹ may be —CH₃. In some embodiments, both P and J may be N.

In some embodiments A- is a Brd4 binding moiety of formula 12:

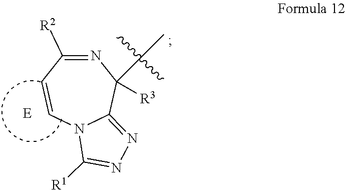

wherein R¹ may be a hydrogen or an optionally substituted alkyl, hydroxyalkyl, aminoalkyl, alkoxyalkyl, halogenated alkyl, hydroxyl, alkoxy, or —COOR⁴; wherein R⁴ may be a hydrogen, or optionally substituted aryl, aralkyl, cycloalkyl, heteroaryl, heteroaralkyl, heterocycloalkyl, alkyl, alkenyl, alkynyl, or cycloalkylalkyl group optionally interrupted by one or more heteroatoms; R² may be an optionally substituted aryl, alkyl, cycloalkyl, or aralkyl group; R³ may be a hydrogen, halogen, or optionally substituted alkyl group (e.g., —(CH₂)ₓ—C(O)N(R²⁰)(R²¹), —(CH₂)ₓ—N(R²⁰C(O)(R²¹), or halogenated alkyl group, wherein x may be an integer from 1 to 10, and R²⁰ and R²¹ may independently be a hydrogen or C₁-C₆ alkyl group (typically R²⁰ may be a hydrogen and R²¹ may be a methyl)); and Ring E may be an aryl, substituted aryl, heteroaryl, arene, substituted arene, or heteroarene group. In some embodiments, x may be an integer from 1 to 6. In some embodiments, x may be an integer from 1 to 3.

In some embodiments, A- is a Brd4 binding moiety of formula 13 or formula 14:

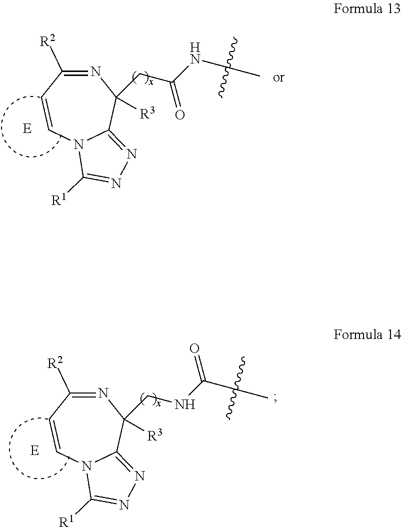

wherein x, R¹, R², R³, and Ring E are as defined herein.

In some embodiments, A- is a thienotriazolodiazepine Brd4 binding moiety. In some embodiments A- is a thienotriazolodiazepine Brd4 binding moiety of formula 15:

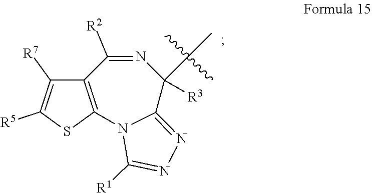

wherein R² may be an aryl group optionally substituted with halogen, —OR⁶, —SR⁶, —N(R⁶)₂, —N(R⁶)COR⁹, or one or more optionally substituted alkyl, alkenyl, alkynyl, aryl, heteroaryl, amino, or amido groups, wherein R⁶ and R⁹ may independently be a hydrogen or alkyl group; R¹ and R³ may independently be a hydrogen or optionally substituted alkyl group; and R⁵ and R⁷ may independently be a hydrogen, alkyl, alkenyl, alkynyl, halogen, —OH, —SH, or —NH₂. In some embodiments, R² may be a phenyl group optionally substituted with one or more alkyl, cyano, halogenated alkyl, alkoxy, hydroxyalkyl, and/or halogen substituents. In some embodiments, R² may be a phenyl group optionally substituted with one or more halogenated alkyl groups. In some embodiments, R² may be a phenyl group optionally substituted with one or more halogens. In some embodiments, R² may be a phenyl group substituted with one, two, three, four or five halogens.

In some embodiments, A- is a thienotriazolodiazepine Brd4 binding moiety of formula 16 or formula 17:

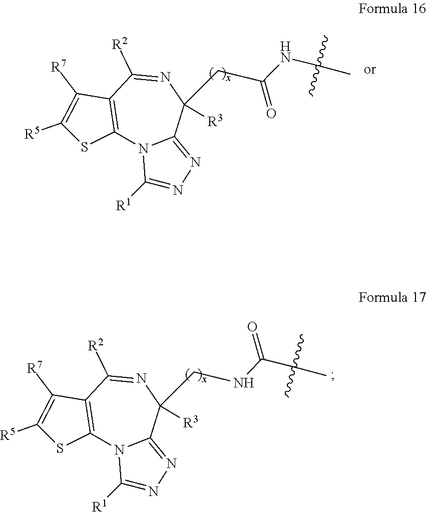

wherein x, R¹, R², R³, R⁵, and R⁷ are as defined herein.

In some embodiments A- is a Brd4 binding moiety of formula 18:

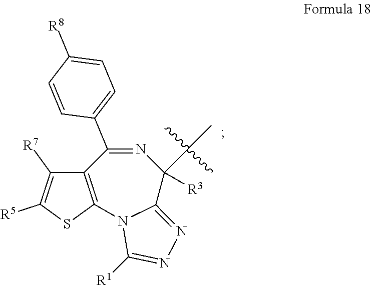

wherein R³ may be a hydrogen or optionally substituted C₁-C₆ alkyl group; R¹, R⁵, and R⁷ are each independently hydrogen, methyl, ethyl, or halomethyl (e.g., trifluoromethyl); and R⁸ may be a halogen, optionally substituted aryl, amino, or amido group. In some embodiments, R³ may be a hydrogen or —CH₃. In some embodiments, R³ may be —CH₃. In some embodiments, R¹, R⁵, and R⁷ are each independently hydrogen, methyl, ethyl, or trifluoromethyl. In some embodiments, R¹, R⁵, and R⁷ are each methyl or ethyl. In some embodiments, R¹, R⁵, and R⁷ are each ethyl. In some embodiments, R¹, R⁵, and R⁷ are each methyl. In some embodiments, R¹, R⁵, and R⁷ are each independently hydrogen. In some embodiments, R¹, R⁵, and R⁷ are each independently trifluoromethyl. In some embodiments, R³ may be hydrogen or —CH₃; R¹, R⁵, and R⁷ may be methyl; and R⁸ may be chloro. In some embodiments, R³ may be hydrogen; R¹, R⁵, and R⁷ may be methyl; and R⁸ may be chloro. In some embodiments, R¹ may be a hydrogen, methyl, ethyl, or halomethyl. In some embodiments, R¹ may be a trifluoromethyl.

In some embodiments, R⁸ may be a halogen. In some embodiments, R⁸ may be —Cl. In some embodiments, R⁸ may be —F. In some embodiments, R⁸ may be a phenyl group optionally substituted with one or more cyano and/or alkoxy groups. In some embodiments, R⁸ may be a phenyl group substituted with a cyano group. In some embodiments, R⁸ may be a phenyl group substituted with a methoxy group. In some embodiments, R⁸ may be an optionally substituted amino group. In some embodiments, R⁸ may be an amino group substituted with an optionally substituted phenyl, benzyl, or heteroaryl group and/or alkyl group. In some embodiments, R⁸ may be an amino group substituted with a phenyl group. In some embodiments, R⁸ may be an amino group substituted with a halogenated phenyl group. In some embodiments, R⁸ may be an amino group substituted with a methyl and a halogenated phenyl group. In some embodiments, R⁸ may be an amino group substituted with a heteroaryl group. In some embodiments, R⁸ may be an amino group substituted with a pyridyl group. In some embodiments, R⁸ may be an amino group substituted with a benzyl group. In some embodiments, R⁸ may be an amido group substituted with an alkyl, aralkyl, or alkaryl group. In some embodiments, R⁸ may be an amido group substituted with an aralkyl group. In some embodiments, R⁸ may be an amido group substituted with —(CH₂)ₜ-phenyl group, wherein t is an integer from 1 to 10. In some embodiments, t may be 1 or 2.

In some embodiments A- is a Brd4 binding moiety of formula 19 or formula 20:

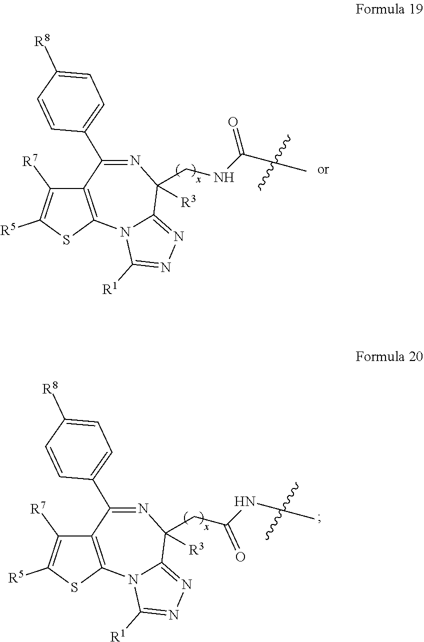

wherein x, R¹, R³, R⁵, R⁷, and R⁸ are as defined herein.

In some embodiments, A- is a Brd4 binding moiety of formula 21:

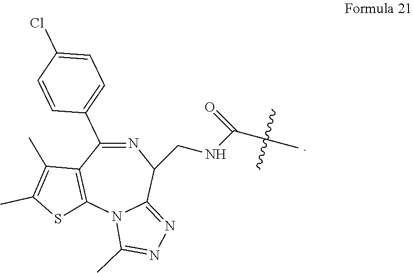 [SMILES: *C(=O)NCC1N=C(c2ccc(Cl)cc2)c2c(sc(C)c2C)-n2c(C)nnc21]

In some embodiments, A- is JQ1 and has the structure of formula 22:

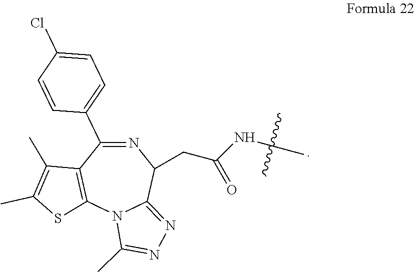 [SMILES: *NC(=O)CC1N=C(c2ccc(Cl)cc2)c2c(sc(C)c2C)-n2c(C)nnc21]

In some embodiments, A- is I-BET762 and has the structure of formula 23:

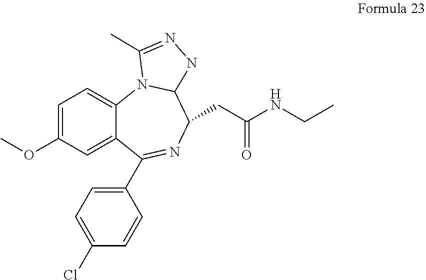 [SMILES: CCNC(=O)C[C@@H]1N=C(c2ccc(Cl)cc2)c2cc(OC)ccc2N2C(C)=NNC12]

In some embodiments, A- includes components C— and —X— (such that C—X—=A-), wherein C— is a bromodomain binding fragment of a bromodomain binding moiety and —X— is a substituted or unsubstituted—O—C₆₋₁₀ arylene, a substituted or unsubstituted —O—5-10 membered heteroarylene, or a substituted or unsubstituted —C₆₋₁₀—OM arylene, wherein M is an alkoxy group. In some embodiments, C—X— has the structure of formula I:

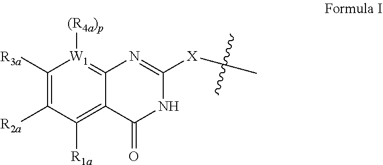

wherein R₁ₐ and R₃ₐ are each independently selected from alkoxy, alkyl, amino, halogen, and hydrogen; R₂ₐ is selected from alkoxy, alkyl, alkenyl, alkynyl, amide, amino, halogen, and hydrogen; W₁ is independently selected from C and N, wherein if W₁ is C, p is 1 and R₄ₐ is H or if W is N, p is 0.

In some embodiments, C—X— has the structure of formula I-1:

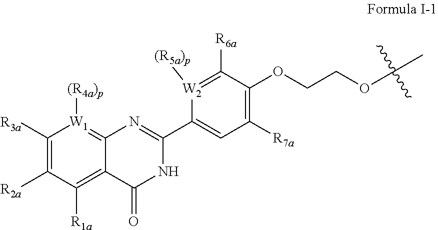

wherein R₆ₐ and R₇ₐ are each independently selected from alkyl, alkoxy, amino, halogen, and hydrogen; R₅ₐ is hydrogen; W₂ is independently selected from C and N, wherein when is W₂ is N, p is 0 or 1 and when W₂ is C, then p is 1.

In some embodiments, C—X— has the structure of formula 9

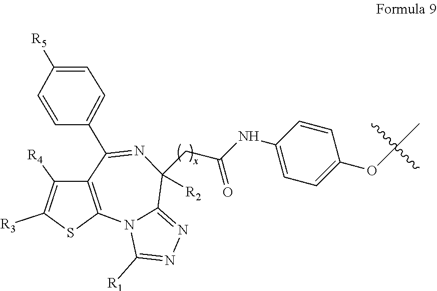

wherein R₁, R₃, and R₄ are each independently selected from hydrogen, methyl, ethyl, and halomethyl; R₂ is selected from hydrogen, C₁-C₆ alkyl group, and substituted C₁-C₆ alkyl group; R₅ is selected from halogen, aryl, substituted aryl, amino, and amido; and x is an integer from 1 to 6.

In some embodiments, C—X has the structure of formula 10:

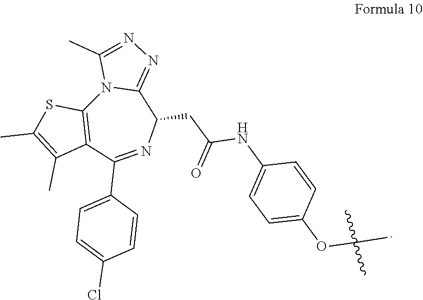 [SMILES: *Oc1ccc(NC(=O)C[C@@H]2N=C(c3ccc(Cl)cc3)c3c(sc(C)c3C)-n3c(C)nnc32)cc1]

In some embodiments, —X— is an unsubstituted —O—C₆₋₁₀ arylene or —O-5-10 membered heteroarylene. In some embodiments, —X— is an unsubstituted —O—C6-10 arylene. In some embodiments, —X— is an unsubstituted —O-5-10 membered heteroarylene. In some embodiments, —X— is a—O—C₆₋₁₀ arylene or —O-5-10 membered heteroarylene substituted with one to five substituents selected from the group consisting of halogen, C1-6 alkyl, C1-6 alkoxy, amino, hydroxy, oxo, and carboxylic acid. In some embodiments, —X— is a —O—C₆₋₁₀ arylene substituted with one to five substituents selected from the group consisting of halogen, C1-6 alkyl, C1-6 alkoxy, amino, hydroxy, oxo, and carboxylic acid. In some embodiments, —X— is a—O-5-10 membered heteroarylene substituted with one to five substituents selected from the group consisting of halogen, C1-6 alkyl, C1-6 alkoxy, amino, hydroxy, oxo, and carboxylic acid. In some embodiments, —X— is an unsubstituted

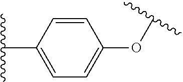 [SMILES: *Oc1ccc(*)cc1]

or a

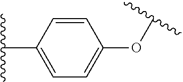 [SMILES: *Oc1ccc(*)cc1]

substituted with one to five substituents selected from the group consisting of halogen, C1-6 alkyl, C1-6 alkoxy, amino, hydroxy, oxo, and carboxylic acid.

In some embodiments, C— is a bromodomain binding fragment of a bromodomain inhibitor. In some embodiments, C— has the structure of formula 19 or formula 20:

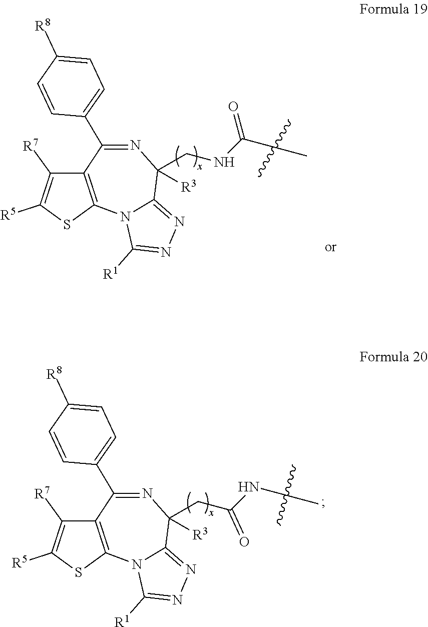

wherein x, R¹, R³, R⁵, R⁷, and R⁸ are as defined herein.

In some embodiments, C— is a bromodomain binding fragment of a BRD4 bromodomain inhibitor having the structure of formula 21:

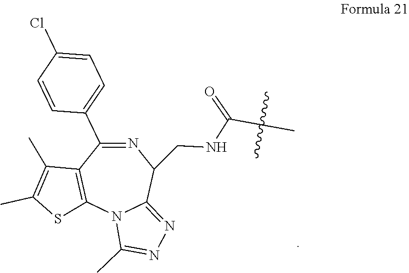 [SMILES: *C(=O)NCC1N=C(c2ccc(Cl)cc2)c2c(sc(C)c2C)-n2c(C)nnc21]

The A- subunit and the —B subunit are commonly joined together by a linker -L- that has a chain having at least 10 contiguous atoms, and commonly at least about 15 contiguous atoms in the backbone chain of the linker. In some embodiments, the linker -L- may desirably have a backbone chain that includes no more than about 50 contiguous atoms in the backbone of the linker, often no more than about 40 contiguous atoms, and in many instances no more than about 30 contiguous atoms in the backbone chain of the linker. It is quite common for the linker -L- to have a backbone chain that includes about 15 to 25 contiguous atoms in the backbone of the linker.

In some embodiments -L- may be a covalent linking group. In some embodiments -L- may be a linker having a contiguous backbone chain which includes at least about 10 atoms. In some embodiments, -L- may have a contiguous backbone chain that includes about 15 to 250 atoms. In some embodiments, -L- may be a combination of one or more optionally substituted arylene, aralkylene, cycloalkylene, heteroarylene, heteroaralkylene, heterocycloalkylene, alkylene, alkenylene, alkynylene, or cycloalkylalkylene, optionally interrupted by one or more heteroatoms, amido, or carboxyl groups. In some embodiments, -L- may include a combination of one or more linking moieties selected from the group consisting of —O—, —(CH₂)ₓ—, —(CH₂CH₂O)_{y}—, —(OCH₂CH₂)_{y}—, —C(O)NR′—, —NR′C(O)—, —C(O)—, —NR*—, and

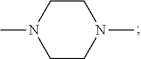

wherein R′ and R* are each independently a hydrogen or C₁-C₆ alkyl; and x and y are each independently an integer from 1 to 10. In some embodiments, R′ may be a hydrogen and R* may be —CH₃.

In some embodiments, -L- may include —(CH₂)ₓ—C(O)N(R′)—(CH₂)_{Q}—N(R*)—(CH₂)_{Q}—N(R′)C(O)—(CH₂)ₓ—C(O)N(R′)—, —(CH₂)ₓ—C(O)N(R′)—(CH₂CH₂O)_{y}—(CH₂)ₓ—C(O)N(R′)—, —C(O)N(R′)—(CH₂)_{Q}—N(R*)—(CH₂)_{Q}—N(R′)C(O)—CH₂)ₓ—, —(CH₂)ₓ—O—(CH₂CH₂O)_{y}—(CH₂)ₓ—N(R′)C(O)—(CH₂)ₓ—, or —N(R′)C(O)—(CH₂)ₓ—C(O)N(R′)—(CH₂)ₓ—O—(CH₂CH₂O)_{y}—(CH₂)ₓ—, wherein R* may be methyl, R′ may be hydrogen, Q may be an integer from 2 to 10, and x and y may independently be an integer from 1 to 10. In some embodiments, R′ may be a hydrogen; R* may be —CH₃; x and y may independently be an integer from 1 to 3; and Q may be 2 or 3.

In some embodiments, -L- may include one or more linking moieties selected from (Gly-Ser-Gly)v and (Gly-Gly-Ser)w, where v and w are typically an integer from 1 to about 10.

In some embodiments, -L- is a polyethylene glycol (PEG) linker. -L- may be a PEG linker selected from the group consisting of (O—CH₂—CH₂)₂ (PEG₂), (O—CH₂—CH₂)₃ (PEG₃), (O—CH₂—CH₂)₄ (PEG₄), (O—CH₂—CH₂)₅ (PEG₅), (O—CH₂—CH₂)₆ (PEG₆), (O—CH₂—CH₂)₇ (PEG₇), (O—CH₂—CH₂)₈ (PEG₈), (O—CH₂—CH₂)₉ (PEG₉), or (O—CH₂—CH₂)₁₀ (PEG₁₀).

In some embodiments, A- is OTX and -L- is a PEG linker, having the structure of formula 24:

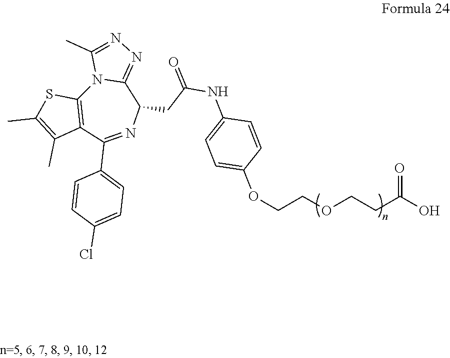

In some embodiments, A- is OTX and -L- is a PEG₅ linker, having the structure of formula 24:

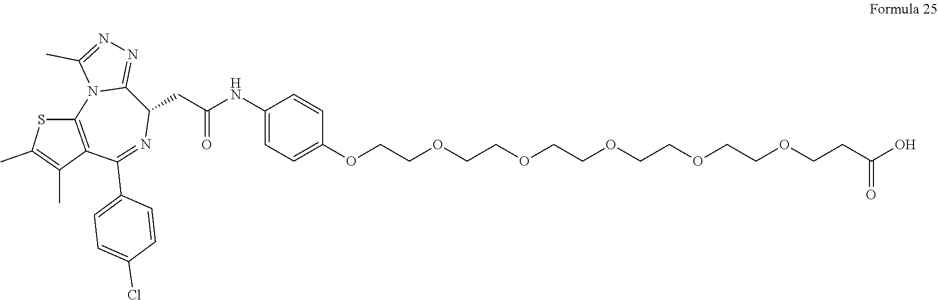 [SMILES: Cc1sc2c(c1C)C(c1ccc(Cl)cc1)=N[C@@H](CC(=O)Nc1ccc(OCCOCCOCCOCCOCCOCCC(=O)O)cc1)c1nnc(C)n1-2]

In some embodiments, A- is JQ1 and -L- is a PEG linker, having the structure of formula 26:

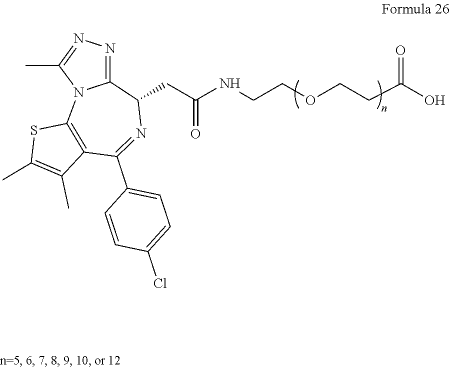

In some embodiments, A- is JQ1 and -L- is a PEG₆ linker, having the structure of formula 27:

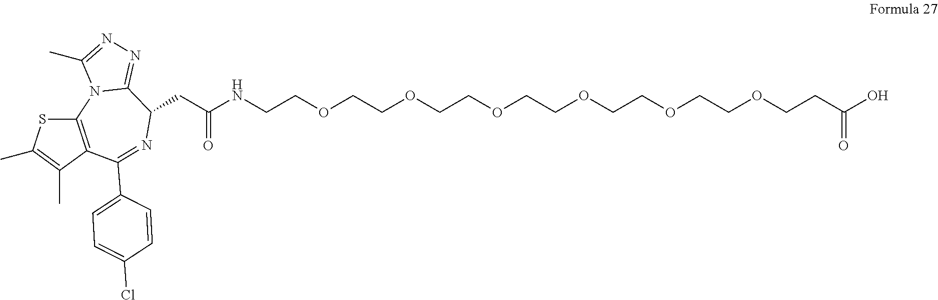 [SMILES: Cc1sc2c(c1C)C(c1ccc(Cl)cc1)=N[C@@H](CC(=O)NCCOCCOCCOCCOCCOCCOCCC(=O)O)c1nnc(C)n1-2]

In some embodiments, A- is JQ1 and -L- is a PEG₈ linker, having the structure of formula 28:

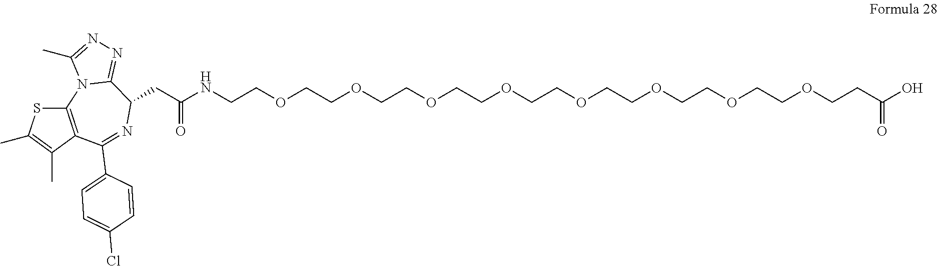 [SMILES: Cc1sc2c(c1C)C(c1ccc(Cl)cc1)=N[C@@H](CC(=O)NCCOCCOCCOCCOCCOCCOCCOCCOCCC(=O)O)c1nnc(C)n1-2]

In some embodiments, A- is JQ1 and -L- is a PEG₁₀ linker, having the structure of formula 29:

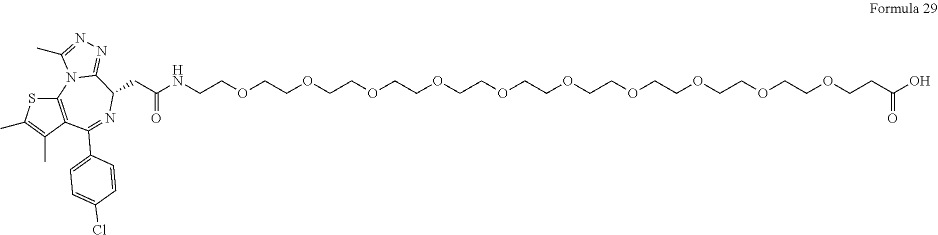 [SMILES: Cc1sc2c(c1C)C(c1ccc(Cl)cc1)=N[C@@H](CC(=O)NCCOCCOCCOCCOCCOCCOCCOCCOCCOCCOCCC(=O)O)c1nnc(C)n1-2]

In some embodiments, A- is RVX and -L- is a PEG linker, having the structure of formula 30:

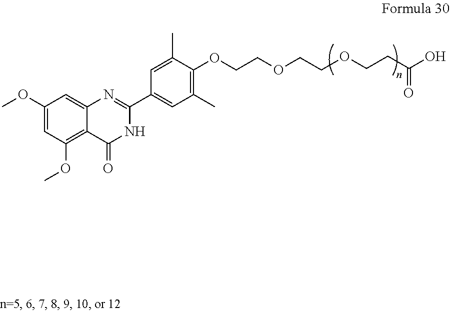

In some embodiments, A- is RVX and -L- is a PEG₅ linker, having the structure of formula 31:

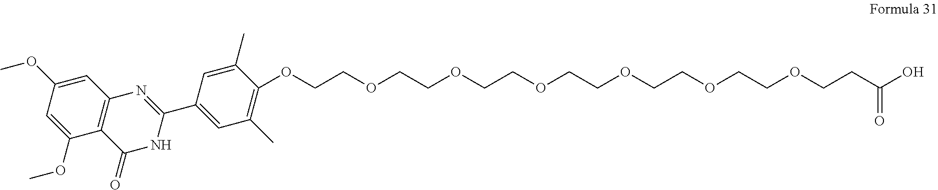 [SMILES: COc1cc(OC)c2c(=O)[nH]c(-c3cc(C)c(OCCOCCOCCOCCOCCOCCOCCC(=O)O)c(C)c3)nc2c1]

In some embodiments, A- is I-BET726 and -L- is a PEG linker, having the structure of formula 32:

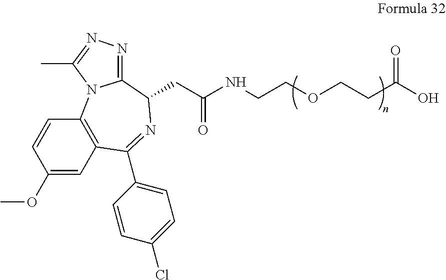

In some embodiments, A- is I-BET726 and -L- is a PEG6 linker, having the structure of formula 33:

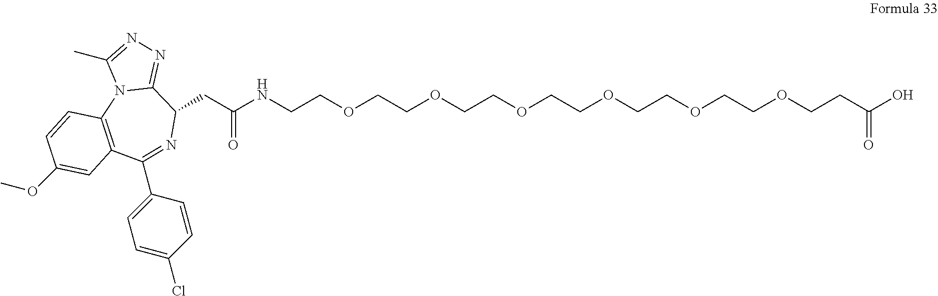 [SMILES: COc1ccc2c(c1)C(c1ccc(Cl)cc1)=N[C@@H](CC(=O)NCCOCCOCCOCCOCCOCCOCCC(=O)O)c1nnc(C)n1-2]

In some embodiments, A- is I-BET726 and -L- is a PEG₈ linker, having the structure of formula 34:

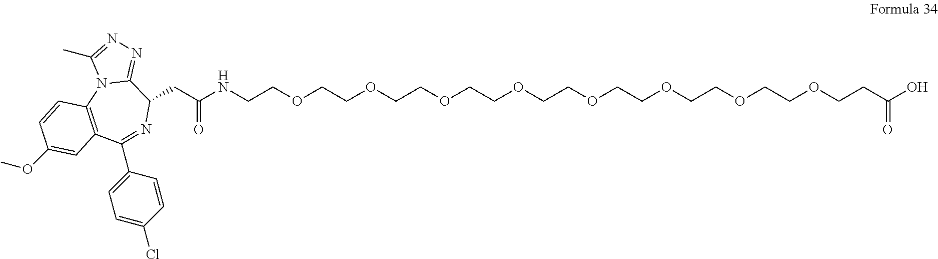 [SMILES: COc1ccc2c(c1)C(c1ccc(Cl)cc1)=N[C@@H](CC(=O)NCCOCCOCCOCCOCCOCCOCCOCCOCCC(=O)O)c1nnc(C)n1-2]

TABLE 2
Summary of exemplary A-L- combinations

| Compound Name | Molecular Formula | Molecular Weight |
|---|---|---|
| JQ1-PEG₆-COOH | C34H46ClN5O9S | 735.27 |
| JQ1-PEG₈-COOH | C38H54ClN5O11S | 823.32 |
| JQ1-PEG₁₀-COOH | C42H62ClN5O13S | 911.38 |
| OTX015-PEG₅-COOH | C38H46ClN5O9S | 783.27 |
| RV208-PEG₅-COOH | C33H46N2O12 | 662.31 |
| I-BET762-PEG₆-COOH | C35H46ClN5O10 | 731.29 |
| I-BET762-PEG₈-COOH | C39H54ClN5O12 | 819.35 |

The A-, -L-, and —B moieties of the synthetic transcription factors and bound together by suitable covalent chemical linkages known in the art. In some embodiments, the A- and -L- moieties are covalently linked by a non-amide bond. In some embodiments, the —B and -L- moieties are covalently linked by a non-amide bond. In some embodiments, the —B and -L- moieties are joined by an ether bond. In some embodiments, both the A- and —B moieties are covalently linked to the -L- moiety by a non-amide bond. As used herein, “non-amide bond” refers to any chemical linkage known in the art and specifically excluding amide bonds. As will be apparent to a skilled artisan, the A-, -L-, and —B moieties may individually include amide bonds yet can still be linked to adjacent moieties by a non-amide bond. Recitation of a covalent linkage by a non-amide bond is not intended as completely excluding amide bonds from the structure of synthetic transcription factors described herein but rather as excluding amide bonds from the covalent bonds between A- and -L- moieties, between -L- and —B moieties, or combinations thereof as specifically recited herein. In some embodiments, the non-amide bond is an ether bond. In some embodiments, the A- and -L- moieties are joined by an ether bond. In some embodiments, the —B and -L- moieties are joined by an amide bond. In some embodiments, the —B and -L- moieties are joined by an ether bond. In some embodiments, both the A- and —B moieties are joined to the -L- moiety by an ether bond.

In some embodiments, the ether bond is a mixed ether having the structure R—O—R″, wherein R is a substituted or unsubstituted arylene, a substituted or unsubstituted 5-10, membered heteroarylene, or a substituted or unsubstituted phenyl of A-, -L-, or —B, and R″ is the adjacent A-, -L-, or —B moiety. In some embodiments, the ether bond is a mixed ether having the structure R—O—R″, wherein R is a substituted or unsubstituted arylene, a substituted or unsubstituted 5-10, membered heteroarylene, or a substituted or unsubstituted phenyl of —X—, and R″ is the adjacent C— or -L moiety.

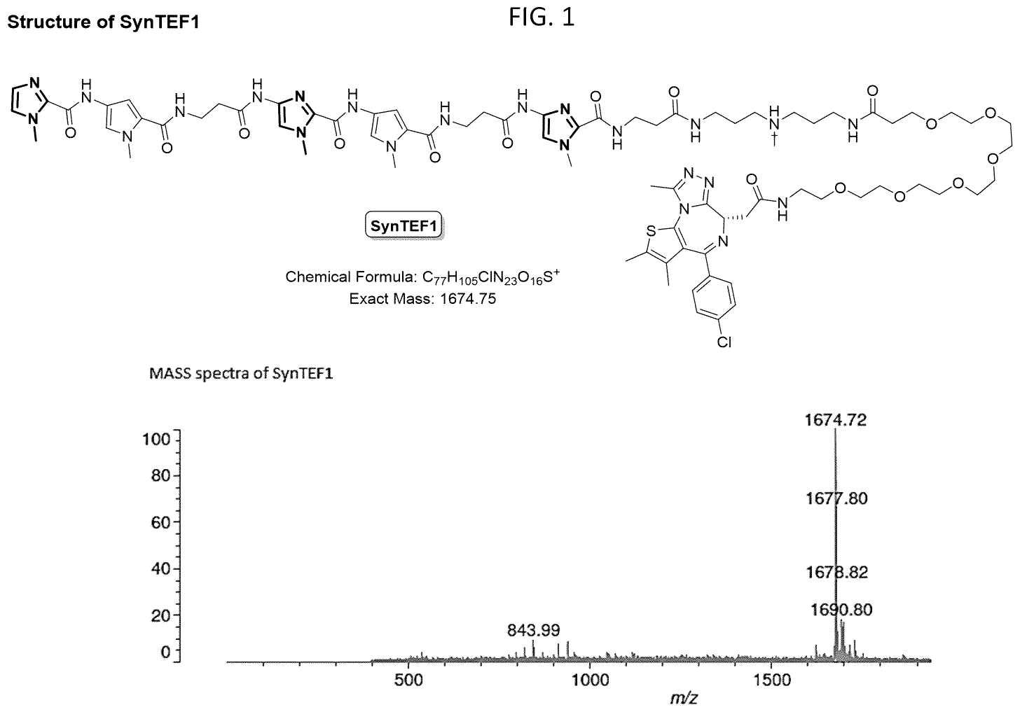

Synthetic transcription factor 1 (SynTEF1, FIG. 1) includes the bromodomain binding moiety (A-) JQ1, a PEG₆ linker (-L-), and the DNA binding moiety PA1 (—B) and was previously described (US Patent Publication 2017/0281643 A1). The synthetic transcription factors described herein are more stable than SynTEF1 while maintaining activity of increasing frataxin expression.

In some embodiments, the synthetic transcription factor is SynTEF3 and has the structure of formula 35:

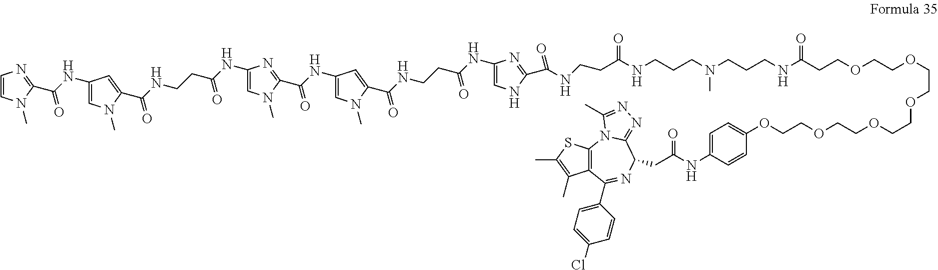 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)c[nH]3)cc1)c1nnc(C)n1-2]

In some embodiments, the synthetic transcription factor is SynTEF4 and has the structure of formula 36:

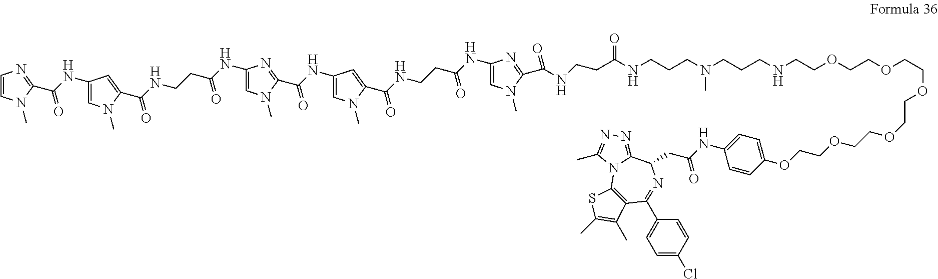 [SMILES: Cc1sc2c(c1C)C(c1ccc(Cl)cc1)=N[C@@H](CC(=O)Nc1ccc(OCCOCCOCCOCCOCCOCCNCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2]

In some embodiments, the synthetic transcription factor is SynTEF5 and has the structure of formula 37:

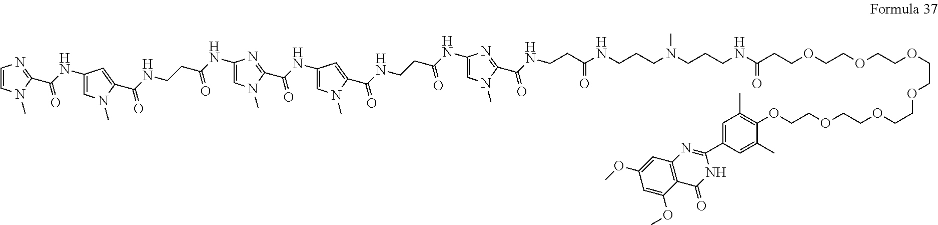 [SMILES: COc1cc(OC)c2c(=O)[nH]c(-c3cc(C)c(OCCOCCOCCOCCOCCOCCOCCC(=O)NCCCN(C)CCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(C)c3)nc2c1]

In some embodiments, the synthetic transcription factor is SynTEF11 and has the structure of formula 38:

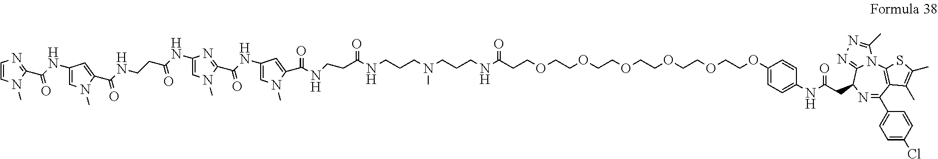 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)CCNC(=O)c3cc(NC(=O)c4nc(NC(=O)CCNC(=O)c5cc(NC(=O)c6nccn6C)cn5C)cn4C)cn3C)cc1)c1nnc(C)n1-2]

In some embodiments, the synthetic transcription factor is SynTEF33 and has the structure of formula 39:

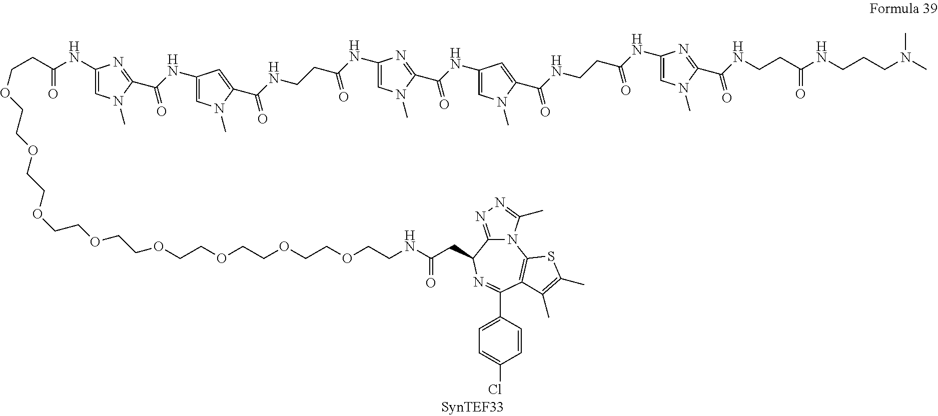 [SMILES: Cc1sc2c(c1C)C(c1ccc(Cl)cc1)=N[C@@H](CC(=O)NCCOCCOCCOCCOCCOCCOCCOCCOCCC(=O)Nc1cn(C)c(C(=O)Nc3cc(C(=O)NCCC(=O)Nc4cn(C)c(C(=O)Nc5cc(C(=O)NCCC(=O)Nc6cn(C)c(C(=O)NCCC(=O)NCCCN(C)C)n6)n(C)c5)n4)n(C)c3)n1)c1nnc(C)n1-2]

In some embodiments, the synthetic transcription factor is SynTEF34 and has the structure of formula 40:

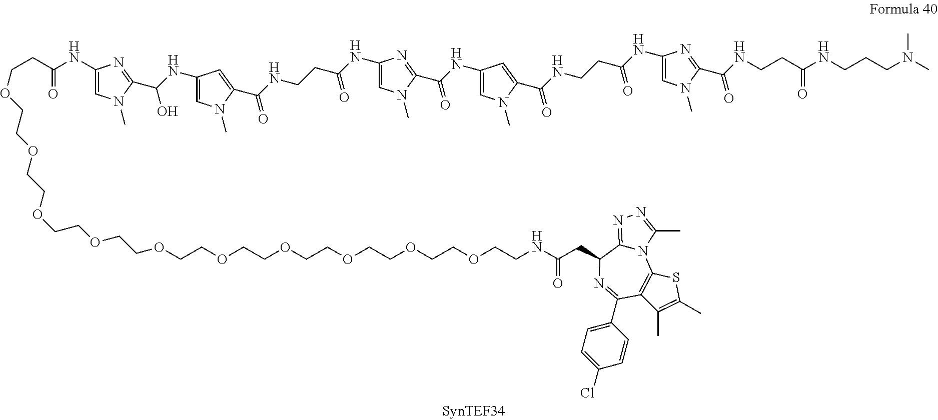 [SMILES: Cc1sc2c(c1C)C(c1ccc(Cl)cc1)=N[C@@H](CC(=O)NCCOCCOCCOCCOCCOCCOCCOCCOCCOCCOCCC(=O)Nc1cn(C)c(C(O)Nc3cc(C(=O)NCCC(=O)Nc4cn(C)c(C(=O)Nc5cc(C(=O)NCCC(=O)Nc6cn(C)c(C(=O)NCCC(=O)NCCCN(C)C)n6)n(C)c5)n4)n(C)c3)n1)c1nnc(C)n1-2]

In some embodiments, the synthetic transcription factor is SynTEF35 and has the structure of formula 41:

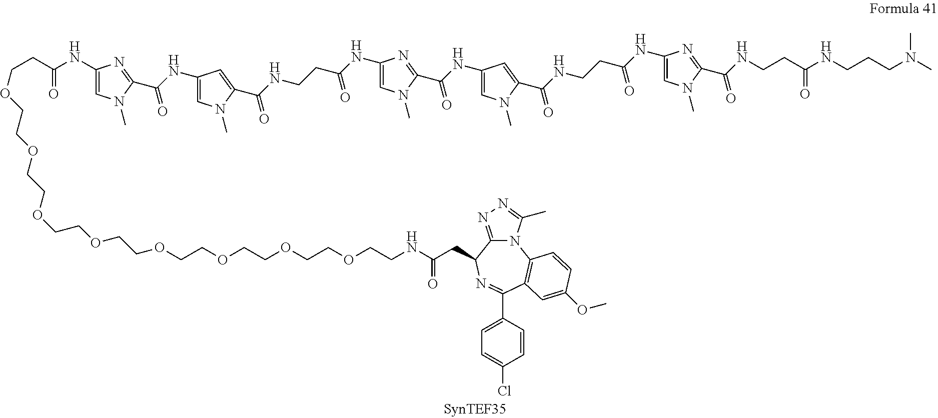 [SMILES: COc1ccc2c(c1)C(c1ccc(Cl)cc1)=N[C@@H](CC(=O)NCCOCCOCCOCCOCCOCCOCCOCCOCCC(=O)Nc1cn(C)c(C(=O)Nc3cc(C(=O)NCCC(=O)Nc4cn(C)c(C(=O)Nc5cc(C(=O)NCCC(=O)Nc6cn(C)c(C(=O)NCCC(=O)NCCCN(C)C)n6)n(C)c5)n4)n(C)c3)n1)c1nnc(C)n1-2]

In some embodiments, the synthetic transcription factor is SynTEF36 and has the structure of formula 42:

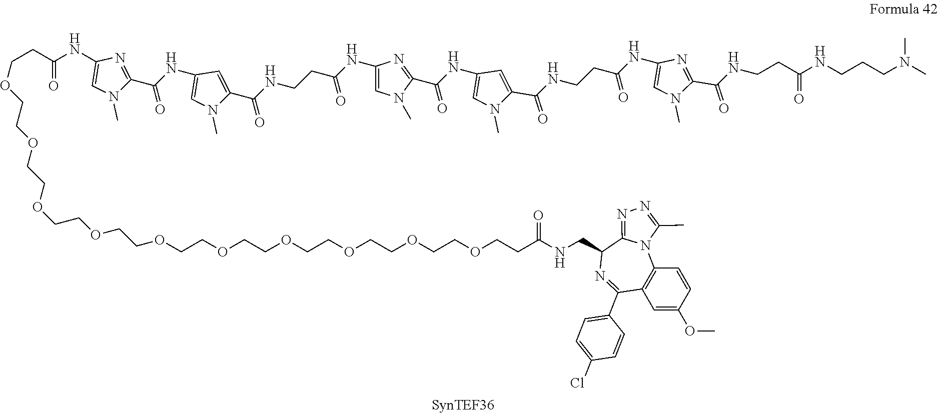 [SMILES: COc1ccc2c(c1)C(c1ccc(Cl)cc1)=N[C@@H](CNC(=O)CCOCCOCCOCCOCCOCCOCCOCCOCCOCCOCCC(=O)Nc1cn(C)c(C(=O)Nc3cc(C(=O)NCCC(=O)Nc4cn(C)c(C(=O)Nc5cc(C(=O)NCCC(=O)Nc6cn(C)c(C(=O)NCCC(=O)NCCCN(C)C)n6)n(C)c5)n4)n(C)c3)n1)c1nnc(C)n1-2]

In some embodiments, the synthetic transcription factor is SynTEF37 and has the structure of formula 43:

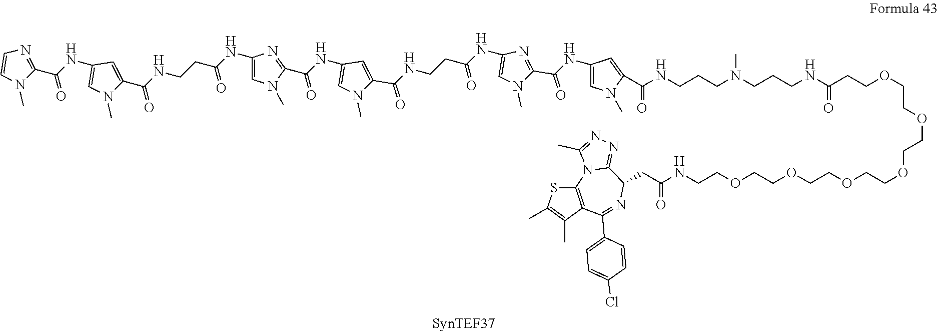 [SMILES: Cc1sc2c(c1C)C(c1ccc(Cl)cc1)=N[C@@H](CC(=O)NCCOCCOCCOCCOCCOCCOCCC(=O)NCCCN(C)CCCNC(=O)c1cc(NC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cn1C)c1nnc(C)n1-2]

In some embodiments, the synthetic transcription factor is SynTEF38 and has the structure of formula 44:

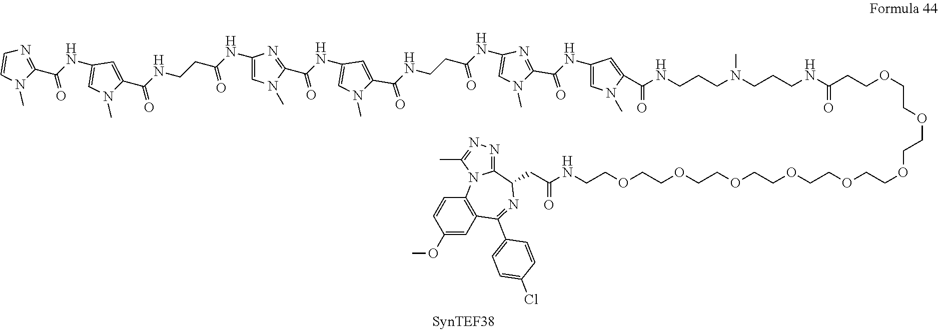 [SMILES: COc1ccc2c(c1)C(c1ccc(Cl)cc1)=N[C@@H](CC(=O)NCCOCCOCCOCCOCCOCCOCCOCCOCCC(=O)NCCCN(C)CCCNC(=O)c1cc(NC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cn1C)c1nnc(C)n1-2]

In some embodiments, the synthetic transcription factor is SynTEF39 and has the structure of formula 45:

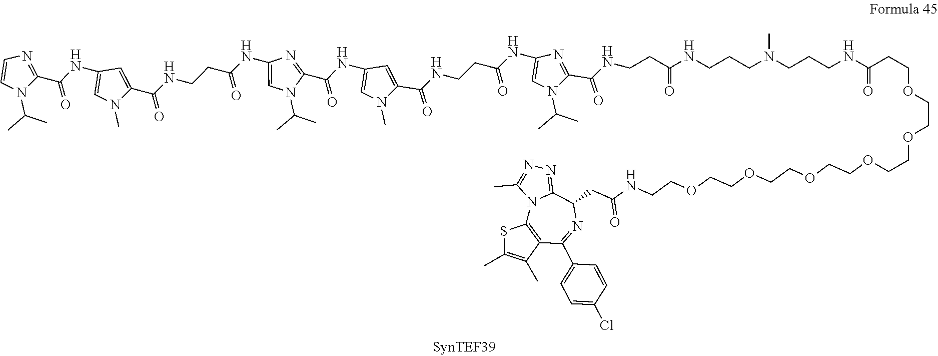 [SMILES: Cc1sc2c(c1C)C(c1ccc(Cl)cc1)=N[C@@H](CC(=O)NCCOCCOCCOCCOCCOCCOCCC(=O)NCCCN(C)CCCNC(=O)CCNC(=O)c1nc(NC(=O)CCNC(=O)c3cc(NC(=O)c4nc(NC(=O)CCNC(=O)c5cc(NC(=O)c6nccn6C(C)C)cn5C)cn4C(C)C)cn3C)cn1C(C)C)c1nnc(C)n1-2]

In some embodiments, the synthetic transcription factor is SynTEF40 and has the structure of formula 46:

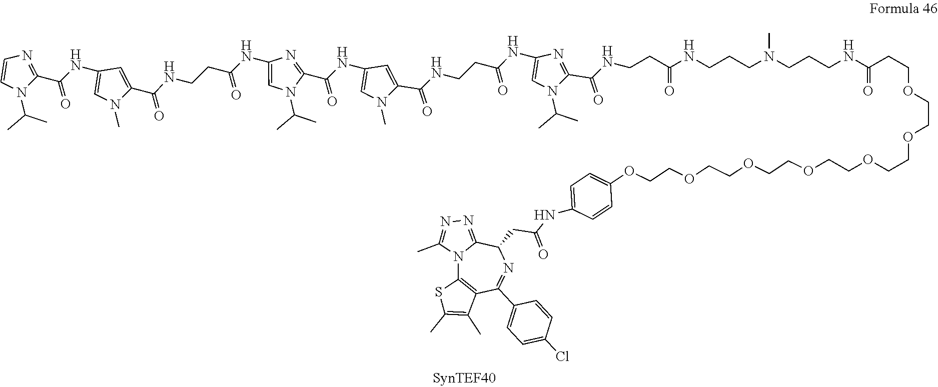 [SMILES: Cc1sc2c(c1C)C(c1ccc(Cl)cc1)=N[C@@H](CC(=O)Nc1ccc(OCCOCCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C(C)C)cn6C)cn5C(C)C)cn4C)cn3C(C)C)cc1)c1nnc(C)n1-2]

In some embodiments, the synthetic transcription factor is SynTEF41 and has the structure of formula 47:

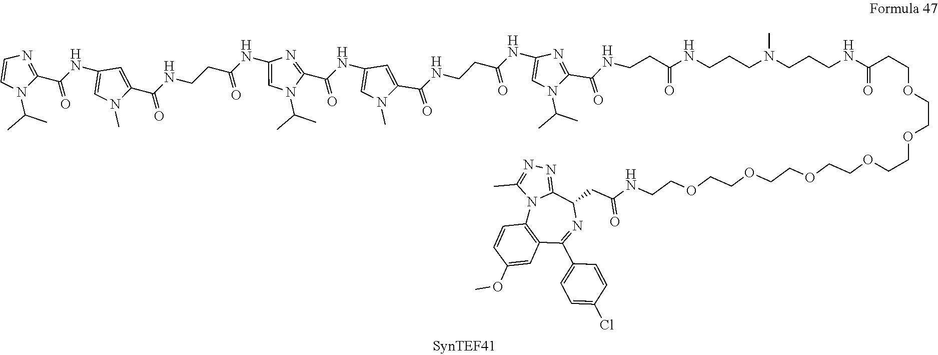 [SMILES: COc1ccc2c(c1)C(c1ccc(Cl)cc1)=N[C@@H](CC(=O)NCCOCCOCCOCCOCCOCCOCCC(=O)NCCCN(C)CCCNC(=O)CCNC(=O)c1nc(NC(=O)CCNC(=O)c3cc(NC(=O)c4nc(NC(=O)CCNC(=O)c5cc(NC(=O)c6nccn6C(C)C)cn5C)cn4C(C)C)cn3C)cn1C(C)C)c1nnc(C)n1-2]

In some embodiments, the synthetic transcription factor is SynTEF42 and has the structure of formula 48:

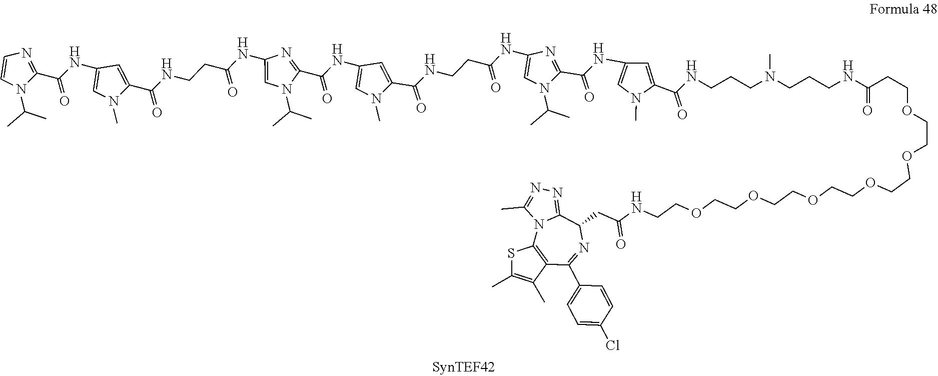 [SMILES: Cc1sc2c(c1C)C(c1ccc(Cl)cc1)=N[C@@H](CC(=O)NCCOCCOCCOCCOCCOCCOCCC(=O)NCCCN(C)CCCNC(=O)c1cc(NC(=O)c3nc(NC(=O)CCNC(=O)c4cc(NC(=O)c5nc(NC(=O)CCNC(=O)c6cc(NC(=O)c7nccn7C(C)C)cn6C)cn5C(C)C)cn4C)cn3C(C)C)cn1C)c1nnc(C)n1-2]

In some embodiments, the synthetic transcription factor is SynTEF43 and has the structure of formula 49.

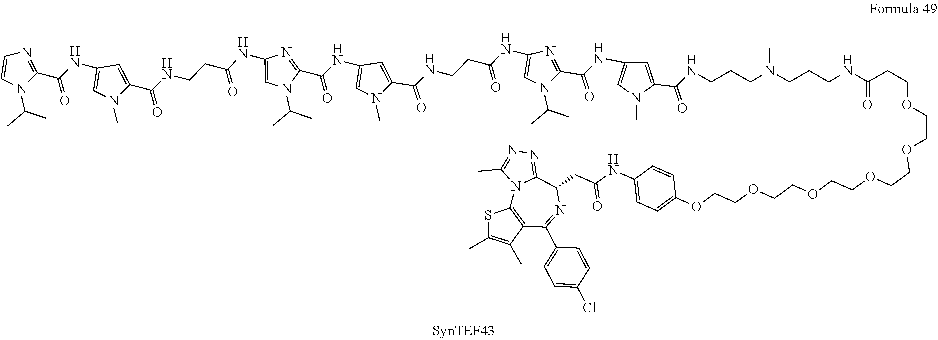 [SMILES: Cc1sc2c(c1C)C(c1ccc(Cl)cc1)=N[C@@H](CC(=O)Nc1ccc(OCCOCCOCCOCCOCCOCCC(=O)NCCCN(C)CCCNC(=O)c3cc(NC(=O)c4nc(NC(=O)CCNC(=O)c5cc(NC(=O)c6nc(NC(=O)CCNC(=O)c7cc(NC(=O)c8nccn8C(C)C)cn7C)cn6C(C)C)cn5C)cn4C(C)C)cn3C)cc1)c1nnc(C)n1-2]

In some embodiments, the synthetic transcription factor is SynTEF44 and has the structure of formula 50:

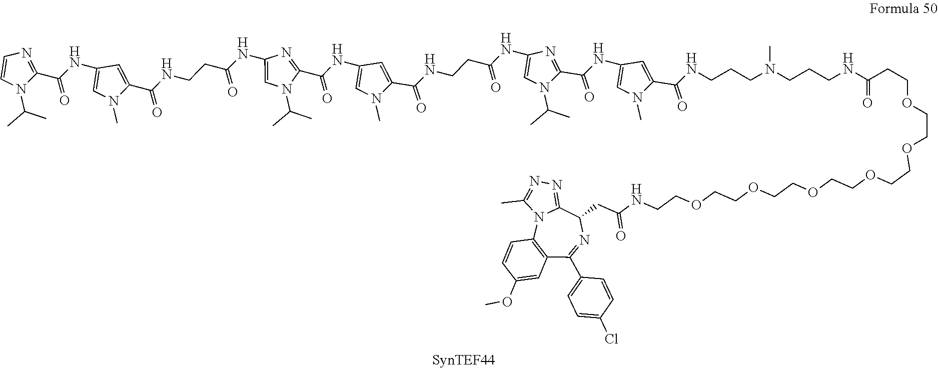 [SMILES: COc1ccc2c(c1)C(c1ccc(Cl)cc1)=N[C@@H](CC(=O)NCCOCCOCCOCCOCCOCCOCCC(=O)NCCCN(C)CCCNC(=O)c1cc(NC(=O)c3nc(NC(=O)CCNC(=O)c4cc(NC(=O)c5nc(NC(=O)CCNC(=O)c6cc(NC(=O)c7nccn7C(C)C)cn6C)cn5C(C)C)cn4C)cn3C(C)C)cn1C)c1nnc(C)n1-2]

In some embodiments, the synthetic transcription factor is SynTEF6 has the structure of formula 51:

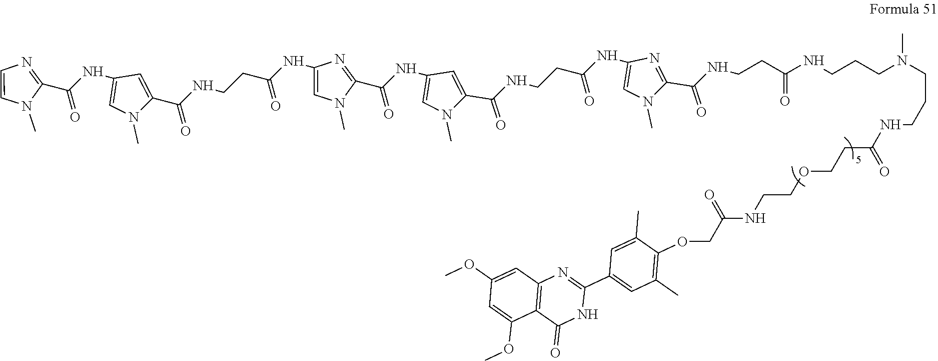

In some embodiments, the synthetic transcription factor is SynTEF10 and has the structure of formula 52:

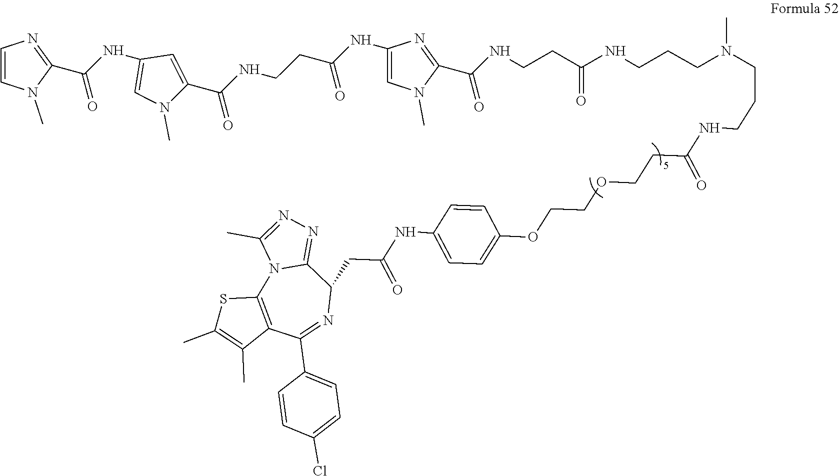

In some embodiments, the synthetic transcription factor is SynTEF12 has the structure of formula 53:

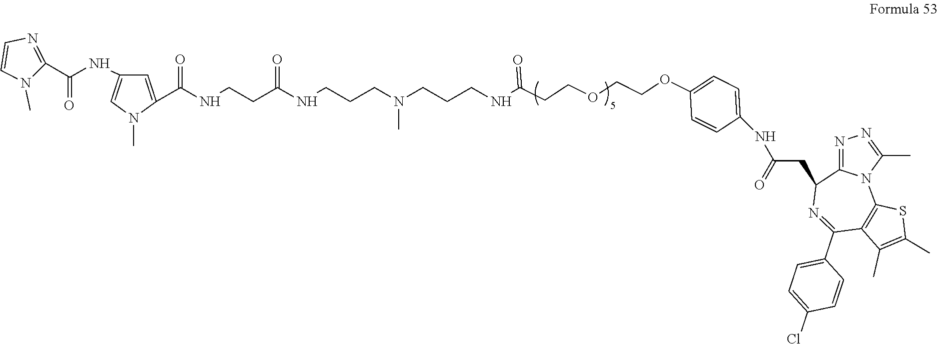

In some embodiments, the synthetic transcription factor is SynTEF14 has the structure of formula 54:

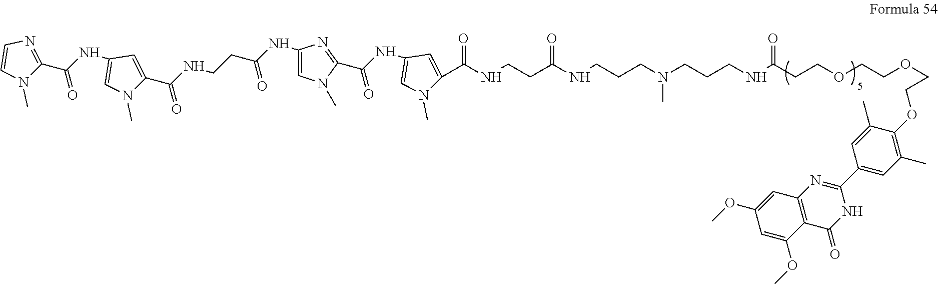

In some embodiments, the synthetic transcription factor is SynTEF13 has the structure of formula 55:

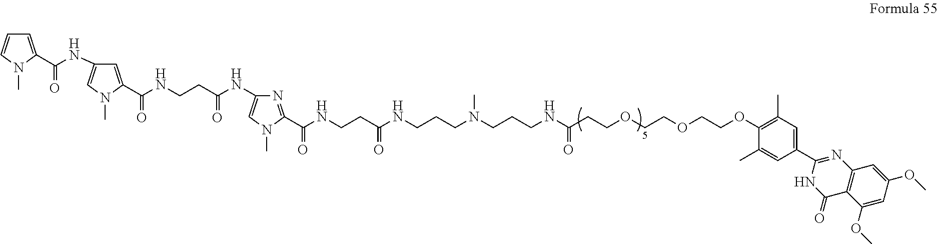

In some embodiments, the synthetic transcription factor has the structure of formula 56:

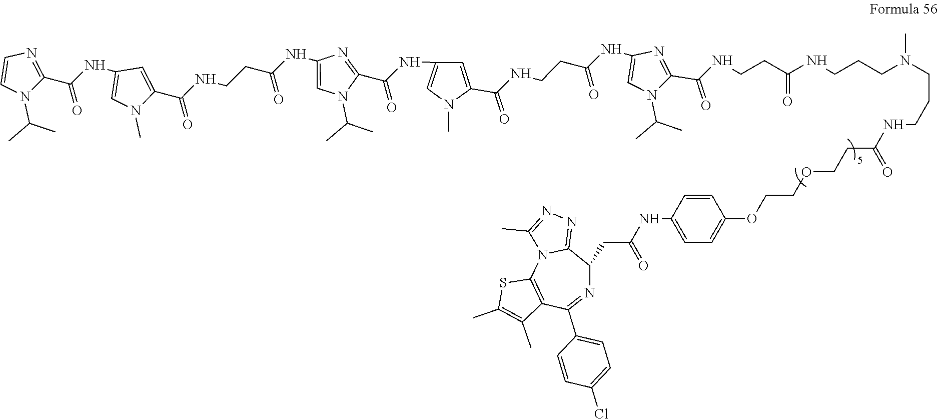

TABLE 3
Summary of structure and naming of synthetic transcription factors.

| Synthetic | | | | Bond | Bond | | | |
|---|---|---|---|---|---|---|---|---|
| Transcription | | | | between | between | | Molecular | Target |
| Factor | A- | -L- | -B | -L-B | A-L- | Molecular Formula | Weight | Ligand |
| SynTEF1 | (+)-JQ-1 | PEG₆ | PA1 | Amide | Amide | C77H104ClN23O16S | 1673.74 | BRD4 |
| SynTEF3 | OTX-015 | PEG₅ | PA1 | Amide | Ether | C81H104ClN23O16S | 1721.74 | BRD4 |
| SynTEF4 | OTX-015 | PEG₅ | PA1 | Ether | Ether | C80H104ClN23O15S | 1693.75 | BRD4 |
| SynTEF5 | RVX-208 | PEG₅ | PA1 | Amide | Ether | C72H96N20O18 | 1528.72 | BRD4 |
| SynTEF11 | OTX-015 | PEG₅ | PA5 | Amide | Ether | C74H95ClN18O14S | 1526.67 | BRD4 |
| SynTEF33 | (+)-JQ-1 | PEG₈ | PA14 | Amide | Amide | C79H108ClN23O18S | 1733.77 | BRD4 |
| SynTEF34 | (+)-JQ-1 | PEG₁₀ | PA14 | Amide | Amide | C83H118ClN23O20S | 1823.83 | BRD4 |
| SynTEF35 | I-BET762 | PEG₆ | PA14 | Amide | Amide | C80H108ClN23O19 | 1729.79 | BRD4 |
| SynTEF36 | I-BET762 | PEG₈ | PA14 | Amide | Amide | C84H116ClN23O21 | 1817.84 | BRD4 |
| SynTEF37 | (+)-JQ1 | PEG₆ | PA16 | Amide | Amide | C80H105ClN24O16S | 1724.75 | BRD4 |
| SynTEF38 | I-BET762 | PEG₈ | PA16 | Amide | Amide | C85H113ClN24O19 | 1808.83 | BRD4 |
| SynTEF39 | (+)-JQ-1 | PEG₆ | PA17 | Amide | Amide | C83H116ClN23O16S | 1757.84 | BRD4 |
| SynTEF40 | OTX-015 | PEG₆ | PA17 | Amide | Ether | C89H120ClN23O17S | 1849.86 | BRD4 |
| SynTEF41 | I-BET762 | PEG₆ | PA17 | Amide | Amide | C83H114ClN23O17 | 1739.85 | BRD4 |
| SynTEF42 | (+)-JQ-1 | PEG₆ | PA18 | Amide | Amide | C86H117ClN24O16S | 1808.85 | BRD4 |
| SynTEF43 | OTX-015 | PEG₆ | PA18 | Amide | Ether | C90H117ClN24O16S | 1856.85 | BRD4 |
| SynTEF44 | I-BET762 | PEG₆ | PA18 | Amide | Amide | C87H117ClN24O17 | 1804.87 | BRD4 |
| SynTEF6 | RVX-208 | PEG₆ | PA1 | Amide | Amide | C78H107N21O20 | 1657.80 | BRD4 |
| SynTEF10 | OTX-015 | PEG₅ | PA4 | Amide | Ether | C67H88ClN17O13S | 1405.62 | BRD4 |
| SynTEF12 | OTX-015 | PEG₅ | PA6 | Amide | Ether | C56H73ClN12O10S | 1140.50 | BRD4 |
| SynTEF14 | RVX-208 | PEG₅ | PA5 | Amide | Ether | C65H89N15O16 | 1335.66 | BRD4 |
| SynTEF13 | RVX-208 | PEG₅ | PA4 | Amide | Ether | C62H88N14O16 | 1284.65 | BRD4 |
| Formula 57 | OTX-015 | PEG₅ | PA17 | Amide | Ether | C87H116ClN23O16S | 1807.51 | BRD4 |

### Therapeutic Methods

In some embodiments, the present disclosure provides a method for modulating transcription of a gene that includes multiple repeats of an oligonucleotide sequence containing 3 to 6 nucleotides, such as a GAA oligonucleotide repeat expansion. Without wishing to be bound by theory, the modulation of transcription is effected by contacting the gene with an agent of the present technology having a formula A-L-B, wherein -L- is a linker; A- is a Brd4 binding moiety; and —B is a polyamide that specifically binds to one or more repeats of the oligonucleotide sequence, thereby modulating the transcription of the gene. In some embodiments, the gene is a frataxin (FXN) gene. In some embodiments, the number of repeats in the oligonucleotide expansion is greater than 50, greater than 70, greater than 100, or in a range of 66-1700.

In some embodiments, the present technology relates to methods and compositions for preventing or treating Friedreich's ataxia in a subject in need thereof. In some embodiments, the methods and compositions of the present technology increase the level of frataxin (FXN) mRNA levels in a cell. In some embodiments, the methods and compositions of the present technology increase frataxin protein levels in a cell. In some embodiments, the methods and compositions of the present technology treat or prevent one or more signs or symptoms of Friedreich's ataxia in a subject. In some embodiments, the methods and compositions of the present technology reduce the likelihood that a subject with risk factors for Friedreich's ataxia will develop one or more signs or symptoms of Friedreich's ataxia, or will delay the onset of Friedreich's ataxia.

According to methods of the present invention, synthetic transcription factors described herein are administered to a subject in need thereof. Subjects in needs of treatment include those already having or diagnosed with Friedreich's ataxia or a sign or symptom of Friedreich's ataxia or a subject who is at risk of developing Friedreich's ataxia.

Friedreich's ataxia (FA or FRDA) is an autosomal recessive neurodegenerative disorder caused by mutations in the FXN gene, which encodes the protein frataxin. Human frataxin is synthesized as a 210-amino acid precursor that is localized to the mitochondrion where the protein is subsequently cleaved to a mature 14 kDa protein (amino acid residues 81-210). FRDA is caused by a hyper-expansion of GAA repeats in the first intron of the FXN gene, resulting in transcriptional repression and insufficient expression of frataxin (FXN), a highly-conserved, iron-binding mitochondrial protein. Transcription is a multistep, highly-regulated process that is divided into three stages: initiation, elongation, and termination. Without wishing to be bound by any particular theory, recent evidence suggests that transcriptional elongation is the primary step affected by the pathological GAA expansion, with the expanded GAA repeats leading to the premature termination or pausing of FXN transcription and, ultimately, decreased cellular frataxin protein levels. Accordingly, without wishing to be bound by any particular theory, FRDA may be characterized as a transcriptional pausing-based genetic disease caused by a defect in transcriptional elongation resulting in transcriptional repression and reduced expression of a gene (e.g., FXN). RNA polymerase-II initiates transcription of the repressed gene underlying the disease, but fails to elongate through the entire open reading frame of the gene to produce full-length pre-mRNA. Splicing is typically unaffected, thereby allowing for the production of normal full-length protein, albeit at reduced levels.

Friedreich's ataxia is the most common hereditary ataxia and causes progressive damage to the nervous system, particularly sensory neurons. Although frataxin is ubiquitously expressed, certain cells (e.g., dorsal root ganglia neurons, cardiomyocytes, and pancreatic beta cells) are particularly sensitive to frataxin depletion, and the resulting degenerative loss of these cells accounts for the clinical manifestations of FRDA. FRDA patients develop neurodegeneration of the large sensory neurons and spinocerebellar tracts, as well as cardiomyopathy and diabetes mellitus. Clinical symptoms of FRDA include ataxia, gait ataxia, muscle weakness, loss of coordination, loss of balance, lack of reflexes in lower limbs, loss of tendon reflexes, loss of ability to feel vibrations in lower limbs, loss of sensation in the extremities, loss of upper body strength, weakness in the arms, spasticity, loss of tactile sensation, impairment of position sense, impaired perception of light touch, impaired perception of pain, impaired perception of temperature, vision impairment, color vision changes, involuntary eye movements, pes cavus, inversion of the feet, hearing impairment, dysarthria, dysphagia, impaired breathing, scoliosis, diabetes, glucose intolerance, carbohydrate intolerance, hypertrophic cardiomyopathy, arrhythmia, myocardial fibrosis, cardiac failure, elevated serum or plasma high sensitive troponin-T (hsTNT) (>14 ng/L), and reduced serum or plasma frataxin protein levels (≤19 ng/ml for pediatric and ≤21 ng/mL for adult patients).

There is an inverse correlation between the number of GAA repeats and FXN protein levels, and there is a tight correlation between frataxin protein levels and the severity of disease. That is, lower frataxin protein levels correlate with greater numbers of GAA repeats and disease severity. FRDA patients exhibiting clinical symptoms have frataxin protein levels that are between 5% and 35% those of healthy individuals. Asymptomatic heterozygous carriers have frataxin mRNA and protein levels that are about 40-50% those of healthy individuals. Most FRDA patients (approximately 98%) carry a homozygous mutation in the first intron of the frataxin (FXN) gene comprising an expansion of a GAA trinucleotide repeat. Pathological GAA expansions can range from about 66 to more than 1,000 trinucleotide repeats, whereas frataxin alleles that are not associated with disease comprise from about 6 to about 34 repeats. Very rare cases of FRDA (about 4%) are characterized by an expansion of a GAA trinucleotide repeat present in one allele and a deleterious point mutation in the other allele. It is generally understood that longer GAA trinucleotide repeats are associated with greater deficiency of frataxin and earlier onset and increased severity of disease. Partially restoring frataxin in affected cells may slow or prevent disease progression.

FRDA is diagnosed by assessing clinical criteria and/or performing genetic testing (Wood, N. W., Arch. Dis. Child., 78:204-207 (1998)). The patient's medical history is evaluated and a physical examination performed. Key to diagnosing FRDA is the recognition of hallmark symptoms, including balance difficulty, loss of joint sensation, absence of reflexes, and signs of neurological problems. In addition, genetic testing can provide a conclusive diagnosis of FRDA.

Clinical Criteria. Strict clinical criteria have been developed that are widely used in the diagnosis of FRDA (Harding, A. E., Brain, 104:589-620 (1981)). Diagnostic criteria include an age of onset before 25 years of age, as well as presence of the following symptoms: progressive ataxia of gait and limbs, absence of knee and ankle jerks, axonal picture on neurophysiology, and dysarthria (if after five years from onset). In over 66% of individuals with FRDA, the following symptoms are present: scoliosis, pyramidal weakness in lower limbs, absence of reflexes in arms, large fiber sensory loss on examination, and abnormal ECG. In less than 50% of individuals having FRDA, the following symptoms are present: nystagmus, optic atrophy, deafness, distal amyotrophy, pes cavus, and diabetes. However, some cases of FRDA present atypically. For example, onset of FRDA may occur over the age of 20 years in some patients. Moreover, some patients retain tendon reflexes.

Core features of pyramidal tract involvement include the association of extensor plantar responses, absence of ankle reflexes, and a progressive course of disease. Pyramidal weakness in lower limbs can lead to paralysis. Skeletal abnormalities are common in FRDA. For example, scoliosis is present in approximately 85% of FRDA patients. Foot abnormalities may be present, including pes cavus and, less frequently, pes planus and equinovarus. Amyotrophy of the lower legs may occur. Optic atrophy is present in about 25% of FRDA cases, while major visual impairment occurs in less than 5% of cases. Deafness is present in less than 10% of FRDA cases. Blood sugar analysis is also performed, as diabetes is seen in approximately 10% of FRDA patients. About 20% of FRDA patients develop carbohydrate intolerance.

A prominent non-neurological feature of FRDA is cardiomyopathy, which may initially present as the sole symptom of disease. An electrocardiogram (ECG) may be performed to assess electrical and muscular functions of the heart. Approximately 65% of FRDA patients present with an abnormal ECG, having widespread T wave inversion in the inferolateral chest leads. The most frequent echocardiographic abnormality in FRDA patients is concentric ventricular hypertrophy. Heart failure typically occurs late in disease progression, often accounting for premature death in FRDA patients.

Within a few years after onset of FRDA, the patient presents with dysarthria and pyramidal weakness, and subsequent nystagmus, which is characterized by involuntary repetitive and jerky eye movements. Within about 10-15 years after onset of disease, the patient becomes wheelchair bound.

Additional tests typically employed to assess FRDA patients include electromyogram (EMG) to measure electrical activity of muscle cells, nerve conduction studies to measure nerve impulse transmission speed, echocardiogram to record the position and motion of heart muscle, and blood tests to determine if the patient has vitamin E deficiency. Magnetic resonance imaging (MRI) or computed tomography (CT) scans provide brain and spinal cord images that can be useful to rule out other neurological conditions.

Genetic Testing. FRDA is a neurological disorder caused by mutations in the frataxin (FXN) gene, having a cytogenetic location of 9q21.11. DNA-based testing is one method that is used to diagnose FRDA. Homozygosity for a GAA repeat expansion in intron 1 of FXN indicates FRDA. Rarely, patients will present as heterozygous for an allele having a GAA repeat expansion and an allele having a point mutation in FXN.

Frataxin protein levels. Frataxin protein levels may be measured to diagnose and monitor treatment efficacy in FRDA patients. This also permits multiplexing with other disease analytes and population screening. In this approach, frataxin protein levels may be measured by a high-throughput immunoassay. Tests can be performed employing whole blood samples or dried blood spots to measure frataxin protein. For whole blood samples, frataxin levels that are ≤19 ng/ml for pediatric individuals (less than 18 years of age) and ≤21 ng/mL for adults (18 years of age or older) are consistent with a diagnosis of FRDA. Frataxin levels that are ≥19 ng/ml for pediatric individuals and ≥21 ng/mL for adults measured using whole blood samples are not consistent with FRDA. For dried blood spot samples, frataxin levels that are ≤15 ng/ml for pediatric individuals (less than 18 years of age) and ≤21 ng/mL for adults are not consistent with FRDA. Frataxin levels that are ≥15 ng/ml for pediatric individuals and ≥21 ng/ml for adults measured using dried blood samples are not consistent with FRDA.

High sensitive Troponin-T. High sensitive Troponin-T (hsTNT) may be useful as a blood biomarker to indicate cumulative myocyte damage leading to fibrosis in FRDA patients (Weidemann, et al., Intl. J. Cardiol., 194:50-57 (2015)). Troponin T is a myofibrillar protein that is present in striated musculature. There are two types of myofilaments, a thick myosin-containing filament and a thin filament consisting of actin, tropomyosin, and troponin. Troponin is a complex of 3 protein subunits: troponin T, troponin I, and troponin C. Troponin T functions to bind the troponin complex to tropomyosin.

In the cytosol, troponin T is present in soluble and protein-bound forms. The soluble or unbound pool of troponin T is released in early stages of myocardial damage. Bound troponin T is released from myofilaments at a later stage of irreversible myocardial damage, corresponding with degradation of myofibrils. The most common cause of cardiac injury is myocardial ischemia (i.e., acute myocardial infarction). Troponin T levels increase approximately 2 to 4 hours after the onset of myocardial necrosis, and can remain elevated for up to 14 days.

Myocardial fibrosis and disease progression appear to correlate strongly with hsTNT levels in FRDA patients. The cutoff point for the hsTNT levels is 14 ng/L (0.014 ng/ml) (ELECSYS® Troponin T hs (TnT-hs), which is available from Roche). Elevated serum or plasma hsTNT levels >14 ng/L (0.014 ng/mL) are seen in FRDA patients with hypertrophic cardiomyopathy (CM). Elevated hsTNT levels may indicate cumulative myocyte damage leading to fibrosis in FRDA.

As used herein, the terms “treat” and “treating” refers to therapeutic and prophylactic measure, wherein the object is to slow down (lessen) an undesired physiological change or pathological disorder resulting from Friedreich's ataxia. For the purposes of this invention, treating Friedreich's ataxia includes, without limitation, alleviating one or more clinical indications, increasing frataxin expression, increasing frataxin mRNA, increasing frataxin protein, decreasing troponin T levels, alleviating cardiomyopathy, and the like. Treating the disease or injury also includes increasing survival of the subject by days, weeks, months, or years as compared to prognosis if treated according to standard medical practice not incorporating administration of the synthetic transcription factors described herein. It is also to be appreciated that the various modes of treatment of medical conditions as described are intended to mean “substantial,” which includes total but also less than total treatment, and wherein some biologically or medically relevant result is achieved. Treating Friedreich's ataxia, as used herein, also refers to treating the signs and symptoms related to reduced frataxin activity or frataxin expression levels characteristic of Friedreich's ataxia. For example, treating Friedreich's ataxia may refer to increasing frataxin mRNA levels in a patient having Friedreich's ataxia relative to the patient prior to treatment. Treating Friedreich's ataxia may also refer to increasing frataxin protein levels in a patient having Friedreich's ataxia relative to the patient prior to treatment.

In some embodiments, following treatment of the subject with a synthetic transcription factor as described here, the subject shows observable and/or measurable reduction in or absence of one or more signs and symptoms of Friedreich's ataxia, such as but not limited to, e.g., ataxia, gait ataxia, muscle weakness, loss of coordination, loss of balance, lack of reflexes in lower limbs, loss of tendon reflexes, loss of ability to feel vibrations in lower limbs, loss of sensation in the extremities, loss of upper body strength, weakness in the arms, spasticity, loss of tactile sensation, impairment of position sense, impaired perception of light touch, impaired perception of pain, impaired perception of temperature, vision impairment, color vision changes, involuntary eye movements, pes cavus, inversion of the feet, hearing impairment, dysarthria, dysphagia, impaired breathing, scoliosis, diabetes, glucose intolerance, carbohydrate intolerance, hypertrophic cardiomyopathy, arrhythmia, myocardial fibrosis, cardiac failure, elevated serum or plasma high sensitive troponin-T (hsTNT) (>14 ng/L), and reduced serum or plasma frataxin levels (≤19 ng/ml for pediatric and ≤21 ng/mL for adult patients).

Subjects in need of treatment can include those already having or diagnosed with a disease or injury as described herein as well as those prone to, likely to develop, or suspected of having a disease or injury as described herein. Pre-treating or preventing a disease or injury according to a method of the present invention includes initiating the administration of the synthetic transcription factors described herein at a time prior to the appearance or onset of Friedreich's ataxia in subjects known to have a mutant frataxin gene or at the initial onset of one or more clinical indications for Friedreich's ataxia. Pre-treating the disorder is particularly applicable to subjects at risk of having or acquiring the disease injury such as individuals known to have a mutant frataxin gene.

As used herein, the terms “prevent” and “preventing” refer to prophylactic or preventive measures intended to inhibit undesirable physiological changes or the development of a clinical indications and symptoms of Friedreich's ataxia. In exemplary embodiments, preventing Friedreich's ataxia comprises initiating the administration of the synthetic transcription factors described herein at a time prior to the appearance or existence of clinical indications, symptoms, pathological features, consequences, or adverse effects associated with Friedreich's ataxia. In such cases, a method of the invention for preventing Friedreich's ataxia comprises administering synthetic transcription factors to a subject in need thereof prior to onset of clinical indications in a subject known to have mutations in the frataxin gene. In some embodiments, a method of the present invention for preventing the progression of Friedreich's ataxia comprises administering synthetic transcription factors to a subject in need thereof following the onset of one or more clinical indications or symptoms of Friedreich's ataxia.

As used herein, the terms “subject” or “patient” are used interchangeably and can encompass any vertebrate including, without limitation, humans, mammals, reptiles, amphibians, and fish. However, advantageously, the subject or patient is a mammal such as a human, or a mammal such as a domesticated mammal, e.g., dog, cat, horse, and the like, or livestock, e.g., cow, sheep, pig, and the like. In exemplary embodiments, the subject is a human. As used herein, the phrase “in need thereof” indicates the state of the subject, wherein therapeutic or preventative measures are desirable. Such a state can include, but is not limited to, subjects having Friedreich's ataxia or a pathological symptom, clinical indication, or feature associated with Friedreich's ataxia.

The agents of the present technology may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regimen).

In some cases, a method of treating or preventing Friedreich's ataxia comprises administering a pharmaceutical composition comprising a therapeutically effective amount of synthetic transcription factor as described herein as a therapeutic agent (i.e., for therapeutic applications). As used herein, the term “pharmaceutical composition” refers to a chemical or biological composition suitable for administration to a mammal. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Examples of compositions appropriate for such therapeutic applications include preparations for parenteral, subcutaneous, transdermal, intradermal, intramuscular, intracoronarial, intramyocardial, intraperitoneal, intravenous or intraarterial (e.g., injectable), or intratracheal administration, such as sterile suspensions, emulsions, and aerosols. Intratracheal administration can involve contacting or exposing lung tissue, e.g., pulmonary alveoli, to a pharmaceutical composition comprising a therapeutically effective amount of synthetic transcription factors as described herein. In some cases, pharmaceutical compositions appropriate for therapeutic applications may be in admixture with one or more pharmaceutically acceptable excipients, diluents, or carriers such as sterile water, physiological saline, glucose or the like. For example, synthetic transcription factors described herein can be administered to a subject as a pharmaceutical composition comprising a carrier solution.

Formulations may be designed or intended for oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. In general, aqueous and non-aqueous liquid or cream formulations are delivered by a parenteral, oral or topical route. In other embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of injectability. The composition should be stable under the conditions of manufacture and storage, and must be shielded from contamination by microorganisms such as bacteria and fungi.

In some embodiments, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

The preferred route may vary with, for example, the subject's pathological condition or age or the subject's response to therapy or that is appropriate to the circumstances. The formulations can also be administered by two or more routes, where the delivery methods are essentially simultaneous or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

In some embodiments, synthetic transcription factors are administered to a subject in need thereof using an infusion, topical application, surgical transplantation, or implantation. In an exemplary embodiments, administration is systemic. In such cases, synthetic transcription factors can be provided to a subject in need thereof in a pharmaceutical composition adapted for intravenous administration to subjects. Typically, compositions for intravenous administration are solutions in sterile isotonic aqueous buffer. The use of such buffers and diluents is well known in the art. Where necessary, the composition may also include a local anesthetic to ameliorate any pain at the site of the injection. Generally, the ingredients are supplied either separately or mixed together in unit dosage form, for example, as a cryopreserved concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In some cases, compositions comprising synthetic transcription factors are lyophilized prior to administration.

In one embodiment, agent of the present technology is administered intravenously. For example, an agent of the present technology may be administered via rapid intravenous bolus injection. In some embodiments, the agent of the present technology is administered as a constant-rate intravenous infusion.

The agent of the present technology may also be administered orally, topically, intranasally, intramuscularly, subcutaneously, or transdermally. In one embodiment, transdermal administration is by iontophoresis, in which the charged composition is delivered across the skin by an electric current.

Other routes of administration include intracranio-ventricular, intracerebroventricularly or intrathecally. Intracerebroventricularly refers to administration into the ventricular system of the brain. Intrathecally refers to administration into the space under the arachnoid membrane of the spinal cord. Thus, in some embodiments, intracerebroventricular or intrathecal administration is used for those diseases and conditions which affect the organs or tissues of the central nervous system.

For systemic, intracerebroventricular, intrathecal, topical, intranasal, subcutaneous, or transdermal administration, formulations of the agents of the present technology may utilize conventional diluents, carriers, or excipients etc., such as those known in the art to deliver the agents of the present technology. For example, the formulations may comprise one or more of the following: a stabilizer, a surfactant, such as a nonionic surfactant, and optionally a salt and/or a buffering agent. The agents of the present technology may be delivered in the form of an aqueous solution, or in a lyophilized form.

Therapeutically effective amounts of synthetic transcription factors are administered to a subject in need thereof. An effective dose or amount is an amount sufficient to effect a beneficial or desired clinical result. With regard to methods of the present invention, the effective dose or amount, which can be administered in one or more administrations, is the amount of synthetic transcription factor sufficient to elicit a therapeutic effect in a subject to whom the agent is administered. The dosage ranges described herein are exemplary and are not intended to be limiting. Dosage, toxicity, and therapeutic efficacy of the agents of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any agent of the present technology used in the methods described herein, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the agent of the present technology, sufficient for achieving a therapeutic or prophylactic effect, ranges from about 0.000001 mg per kilogram body weight per day to about 10,000 mg per kilogram body weight per day. In some embodiments, the dosage ranges will be from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the agent of the present technology ranges from 0.1-10,000 micrograms per kg body weight. An exemplary treatment regimen entails administration once per day or once a week. Intervals can also be irregular as indicated by measuring blood levels of glucose or insulin in the subject and adjusting dosage or administration accordingly. In some methods, dosage is adjusted to achieve a desired fasting glucose or fasting insulin concentration. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of the agent of the present technology is defined as a concentration of the agent of the present technology at the target tissue of 10⁻¹¹ to 10⁻⁶ molar, e.g., approximately 10⁻⁷ molar. This concentration may be delivered by systemic doses of 0.01 to 100 mg/kg or equivalent dose by body surface area. The schedule of doses is optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and the presence of other diseases. Moreover, treatment of a subject with a therapeutically effective amount of the therapeutic compositions described herein can include a single treatment or a series of treatments.

In some embodiments, the agent is provided at a level sufficient to bind at least 2, 3, or more repeats of the oligonucleotide sequence. In some embodiments, of the methods disclosed herein, the cells are contacted with one or more agents of the present technology at a concentration of about 10 nM. In some embodiments, the cells are contacted with one or more agents at a concentration of about 50 nM. In some embodiments, the cells are contacted with one or more agents at a concentration of about 100 nM. In some embodiments, the cells are contacted with one or more agents at a concentration of about 200 nM. In some embodiments, the cells are contacted with one or more agents at a concentration of about 300 nM. In some embodiments, the cells are contacted with one or more agents at a concentration of about 400 nM. In some embodiments, the cells are contacted with one or more agents at a concentration of about 500 nM. In some embodiments, the cells are contacted with one or more agents at a concentration of about 600 nM. In some embodiments, the cells are contacted with one or more agents at a concentration of about 700 nM. In some embodiments, the cells are contacted with one or more agents at a concentration of about 800 nM. In some embodiments, the cells are contacted with one or more agents at a concentration of about 900 nM. In some embodiments, the cells are contacted with one or more agents at a concentration of about 1 μM. In some embodiments, the cells are contacted with one or more agents at a concentration of about 2 μM. In some embodiments, the cells are contacted with one or more agents at a concentration of about 3 μM. In some embodiments, the cells are contacted with one or more agents at a concentration of about 4 μM. In some embodiments, the cells are contacted with one or more agents at a concentration of about 5 μM.

In some embodiments, of the methods disclosed herein, the cells are harvested for subsequent measurement of mRNA and/or protein levels at about 6 hours after having been contacted with one or more doses of the agents of the present technology. In some embodiments, of the methods disclosed herein, the cells are harvested for subsequent measurement of mRNA and/or protein levels at about 12 hours after having been contacted with one or more doses of the agents of the present technology. In some embodiments, of the methods disclosed herein, the cells are harvested for subsequent measurement of frataxin mRNA and/or protein levels at about 24 hours after having been contacted with one or more doses of the agents of the present technology. In some embodiments, the cells are harvested at about 2 days after having been contacted with one or more doses of the agents of the present technology. In some embodiments, the cells are harvested at about 3 days after having been contacted with one or more doses of the agents of the present technology. In some embodiments, the cells are harvested at about 4 days after having been contacted with one or more doses of the agents of the present technology. In some embodiments, the cells are harvested at about 5 days or more after having been contacted with one or more doses of the agents of the present technology.

Following treatment according to the methods provided herein, the treated subject can be monitored for any positive or negative change in one or more clinical indications of Friedreich's ataxia. In some embodiments, the level of frataxin mRNA or frataxin protein is monitored and an increase in frataxin mRNA or protein expression indicates treatment of the subject. In some embodiments the level of troponin T is monitored and a decrease in troponin T indicates treatment of the subject. In some embodiments, a subject's ataxia is monitored and an improvement in the subject's coordination and control of bodily movements indicates treatment of the subject. In some embodiments, an electrocardiogram (ECG) readout is used to monitor a subject's treatment and a normal ECG readout indicates that the subject has been treated.

The present invention has been described in terms of one or more preferred embodiments, and it should be appreciated that many equivalents, alternatives, variations, and modifications, aside from those expressly stated, are possible and within the scope of the invention.

### EXAMPLE 1

The embodiment described here demonstrates the synthesis and activity of synthetic transcription factors specific to GAA repeat sequences.

### Synthesis of SynTEF3

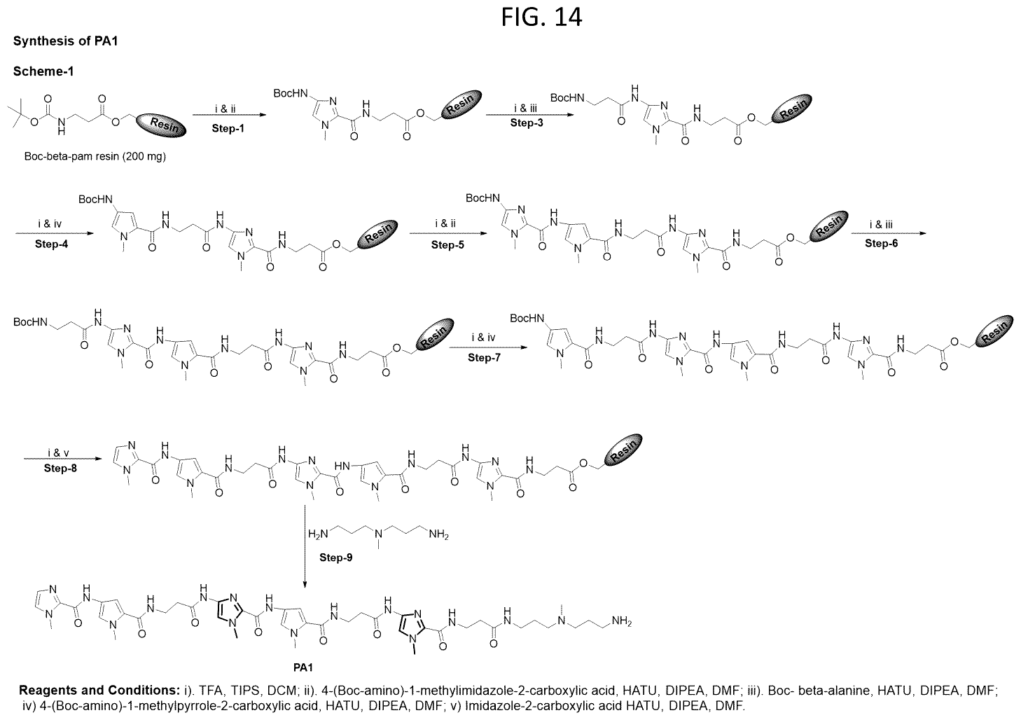

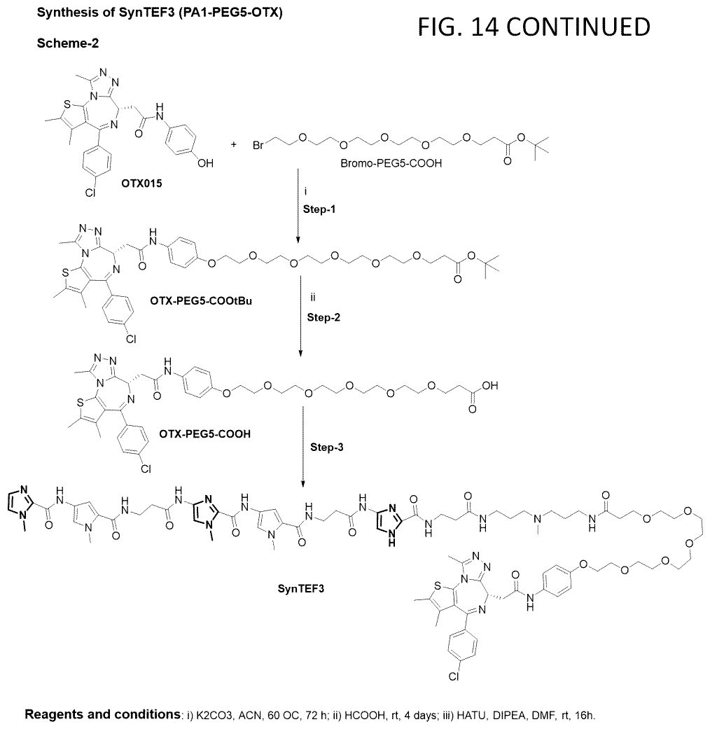

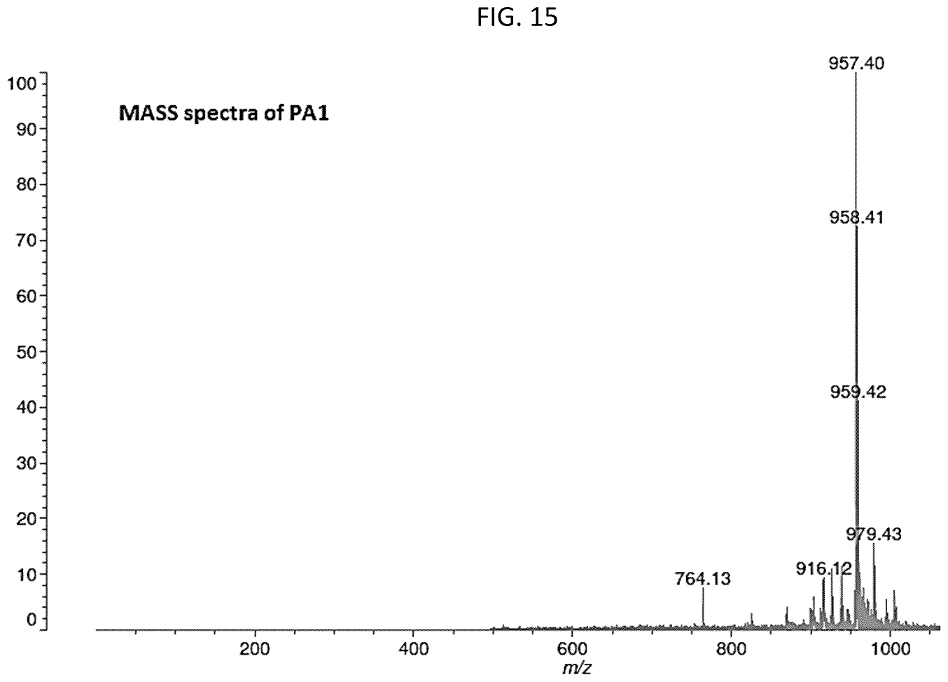

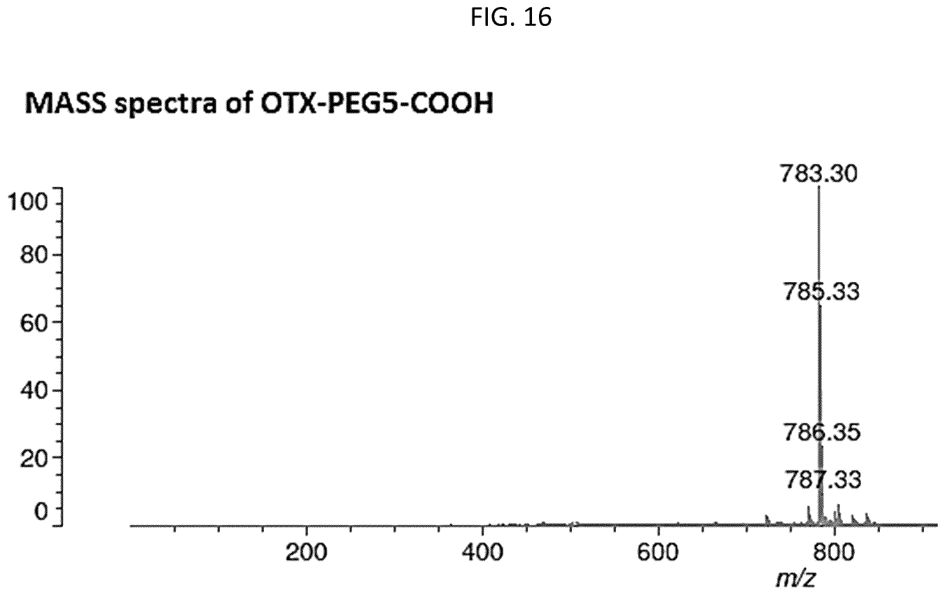

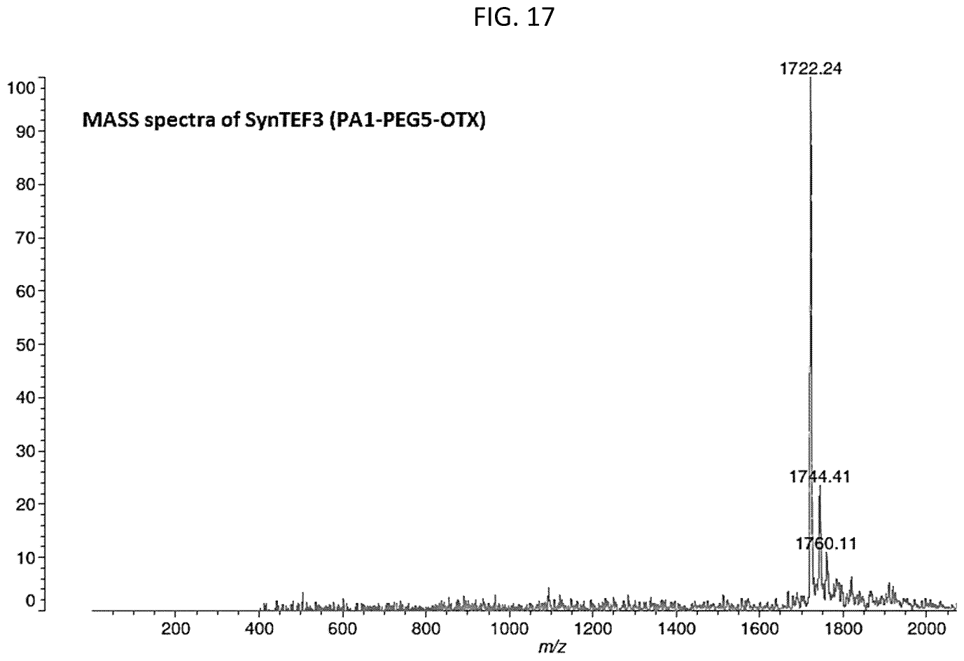

The full synthesis of SynTEF3 is shown in Scheme 1 and Scheme 2 of FIG. 14. For Scheme 1, reagents and conditions are as follows: i) TFA, TIPS, DCM; ii) 4-(Boc-amino)-1-methylimidazole-2-carboxylic acid, HATU, DIPEA, DMF; iii) Boc-β-alanine, HATU, DIPEA, DMF; iv) 4-(Boc-amino)-1-methylpyrrole-2-carboxylic acid, HATU, DIPEA, DMF; v) Imidazole-2-carboxylic acid HATU, DIPEA, DMF. For Boc deprotection, a mixture of 2 ml of TFA, 0.25 ml of DCM and 0.25 ml of TIPS was added to the resin (200 mg) and reaction mixture was stirred for 30 min. For imidazole coupling, a mixture of Boc-Imidazole acid (60 mg), HATU (140 mg) and DIPEA (0.06 ml) in DMF (1 ml) was added to the Boc deprotected resin and the reaction mixture was stirred for 2 h. For pyrrole coupling a mixture of Boc-Pyrrole-2-carboxylic acid (60 mg), HATU (140 mg) and DIPEA (0.06 ml) in DMF (1 ml) was added to the Boc deprotected resin and the reaction mixture was stirred for 2 h.

For Scheme 2, reagents and conditions are as follows: i) K₂CO₃, ACN, 60 OC, 72 h; ii) HCOOH, rt, 4 days; iii) HATU, DIPEA, DMF, rt, 16 h. For step 1 of Scheme 2, K₂CO₃ was added to a stirred solution OTX015 (50 mg) in acetonitrile (2 ml) at 0° C. and stirred for 10 min at same temperature. After 10 min, the solution of Br-PEG₅-COOtBu (51 mg) was added to the reaction mixture then the reaction stirred for 3 days at 60° C. The reaction was monitored by TLC. After completion of the starting materials, reaction mixture was concentrated and purified by HPLC chromatography to give a pure pale yellow compound as a semi solid (45 mg, yield: 53%). For step 2 of Scheme 2, formic acid (2 ml) was added to compound OTX-PEG₅-COOtBu (40 mg) at room temperature and the reaction mixture was stirred for four days at room temperature. After completion of the reaction, reaction mixture was concentrated and co-distilled with ether 3-4 times to give pale yellow color compound as a solid (25 mg, yield: 67%). For step 3, of Scheme 2, PA1 (6 mg) and HATU (30 mg) was added to a stirred solution of OTX-PEG₅-COOH (5 mg) in DMF (2 ml) followed by DIPEA (16 μL) at room temperature. The reaction was stirred at room temperature for 16 h. After 16 h, reaction mixture was dissolved in 15% acetonitrile in water (0.1% TFA) and purified by reverse-phase preparative HPLC on a C18 column. Fractions that showed pure polyamide were frozen in liquid nitrogen and lyophilized to afford a white or off-white powder (4 mg, yield: 36%).

### Synthesis of SynTEF4

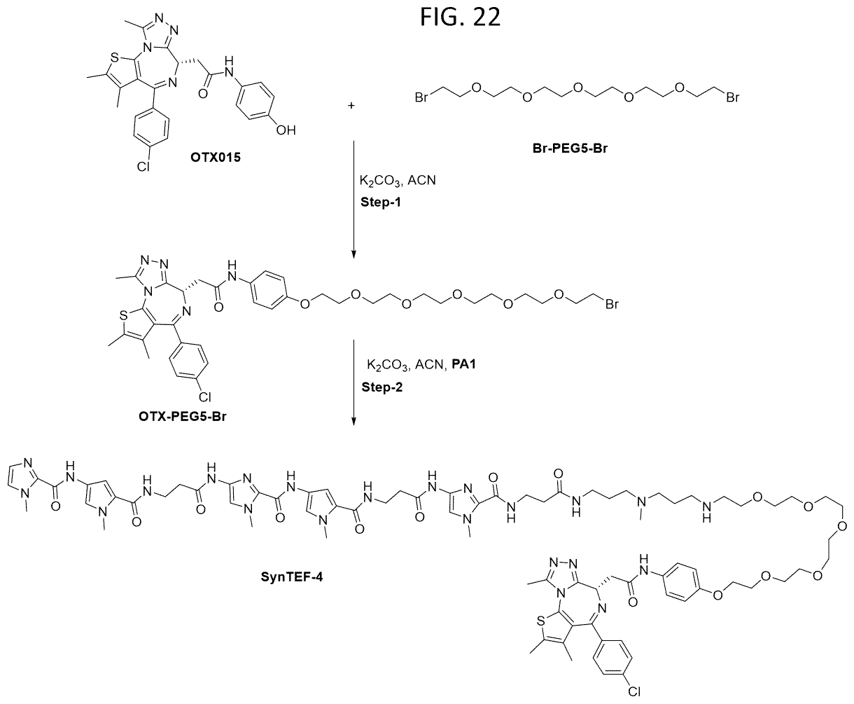

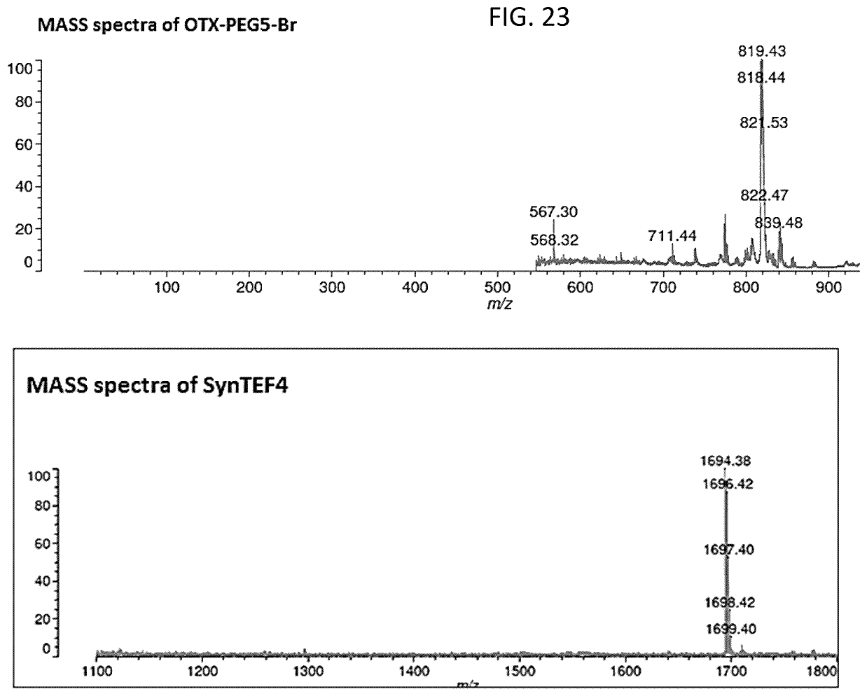

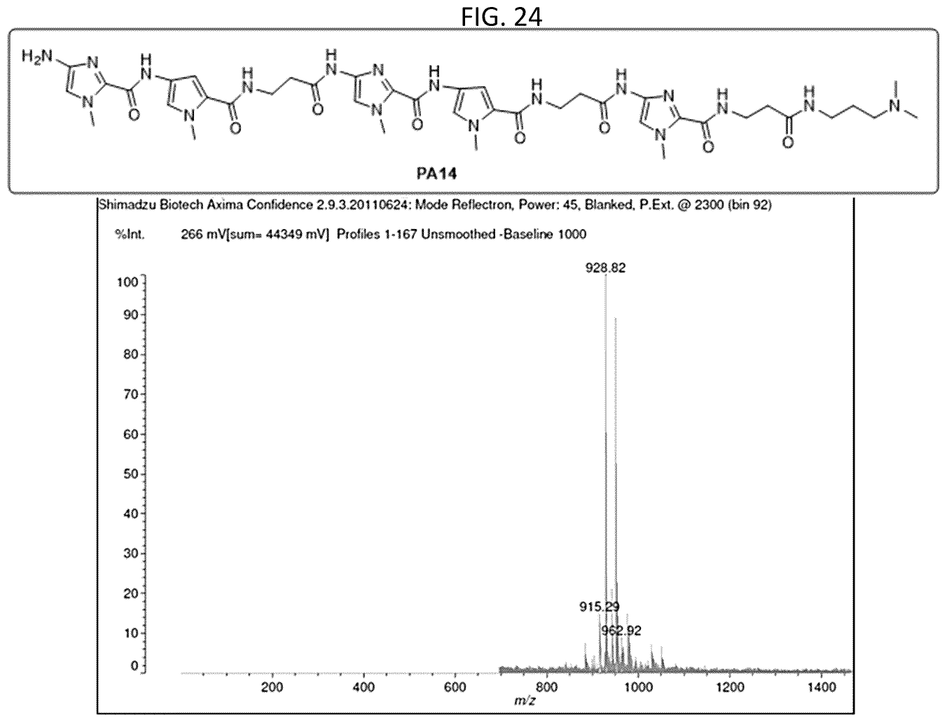

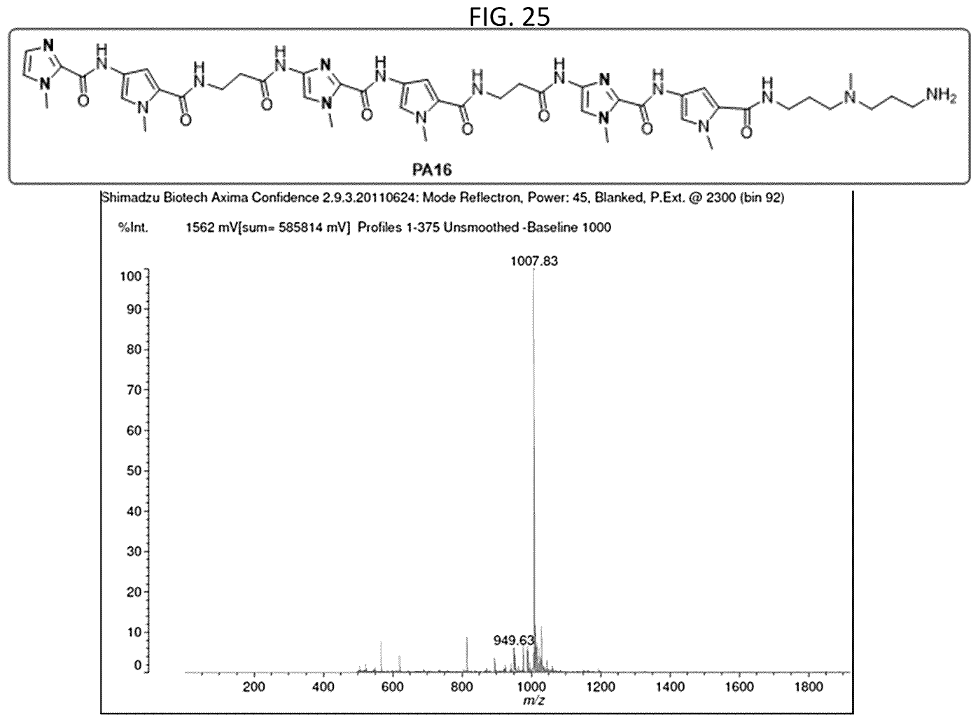

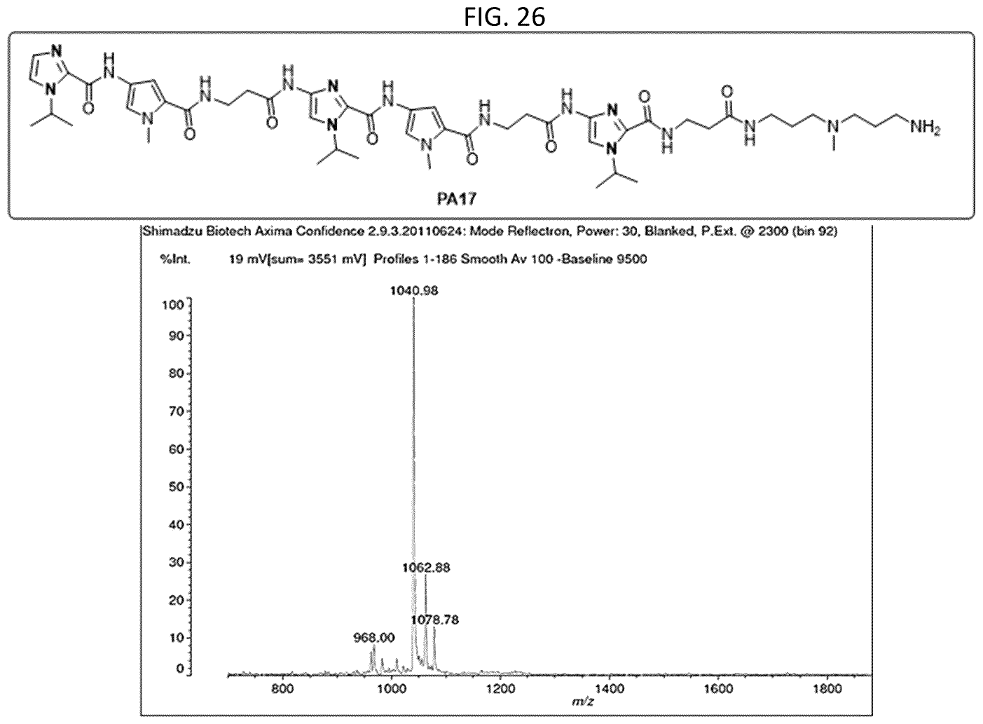

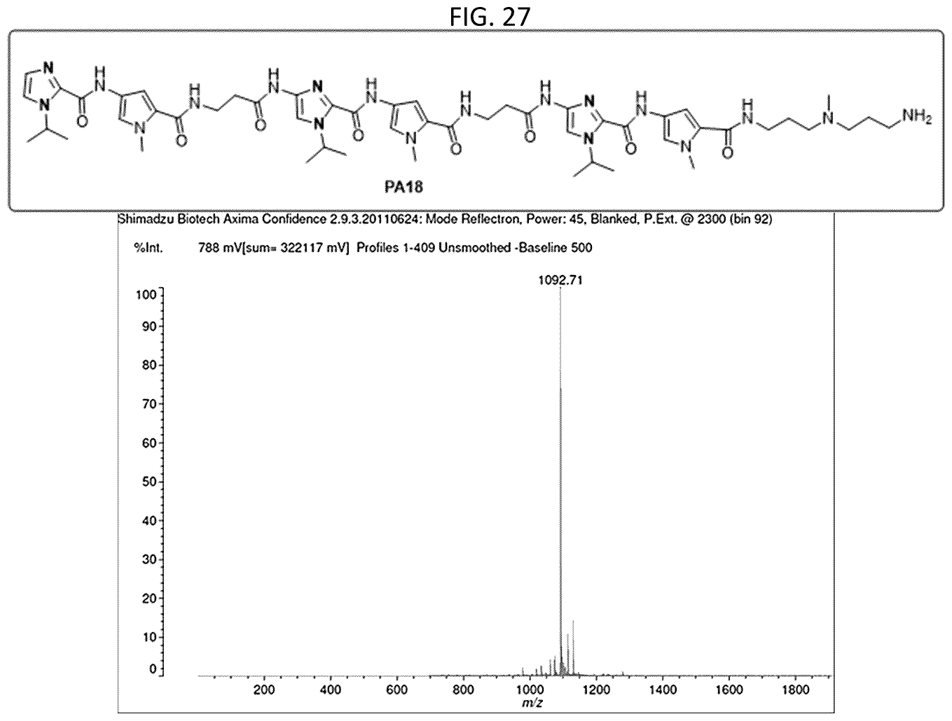

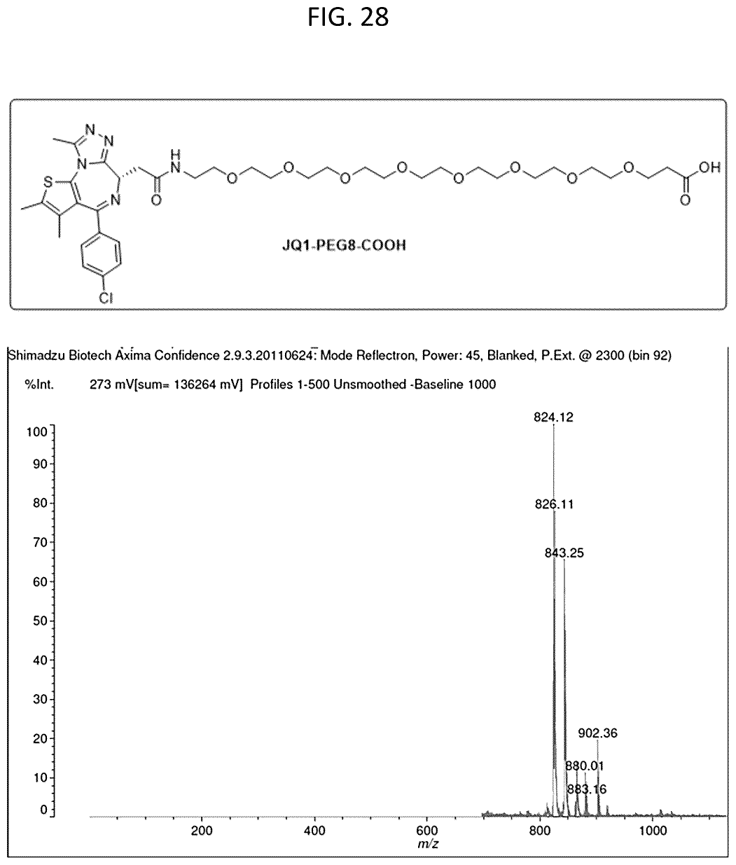

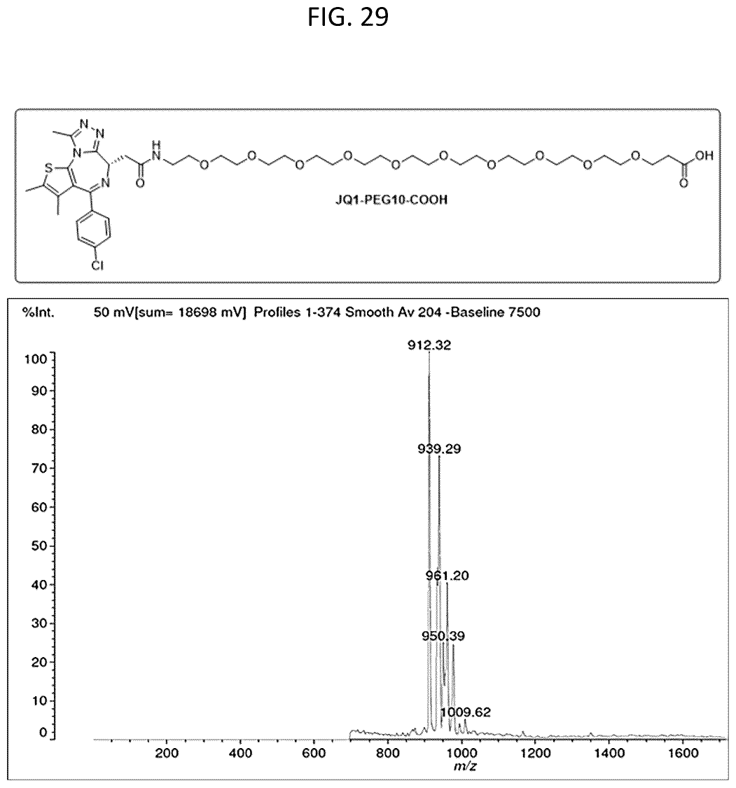

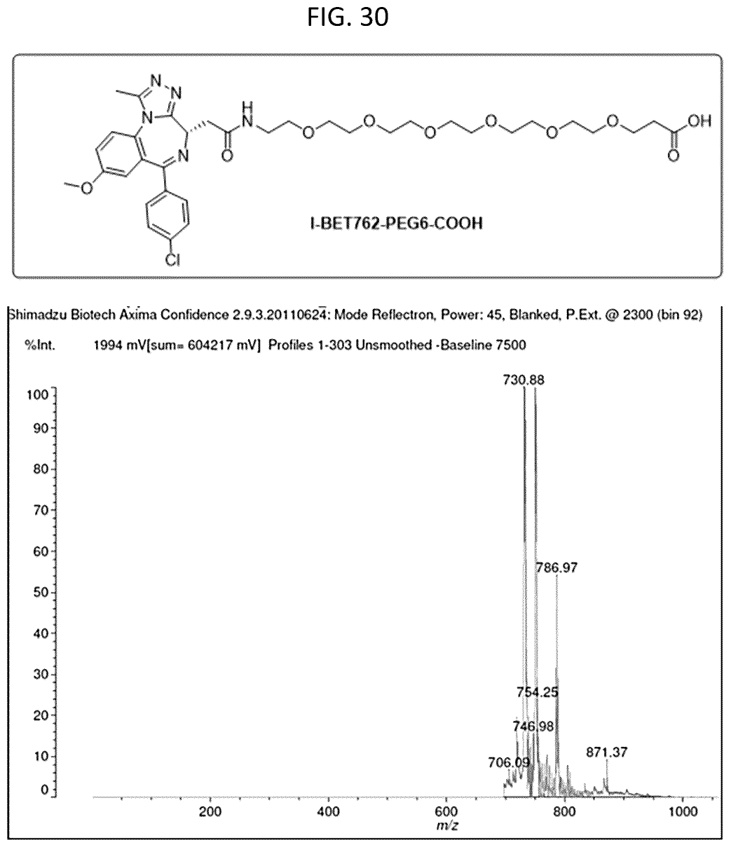

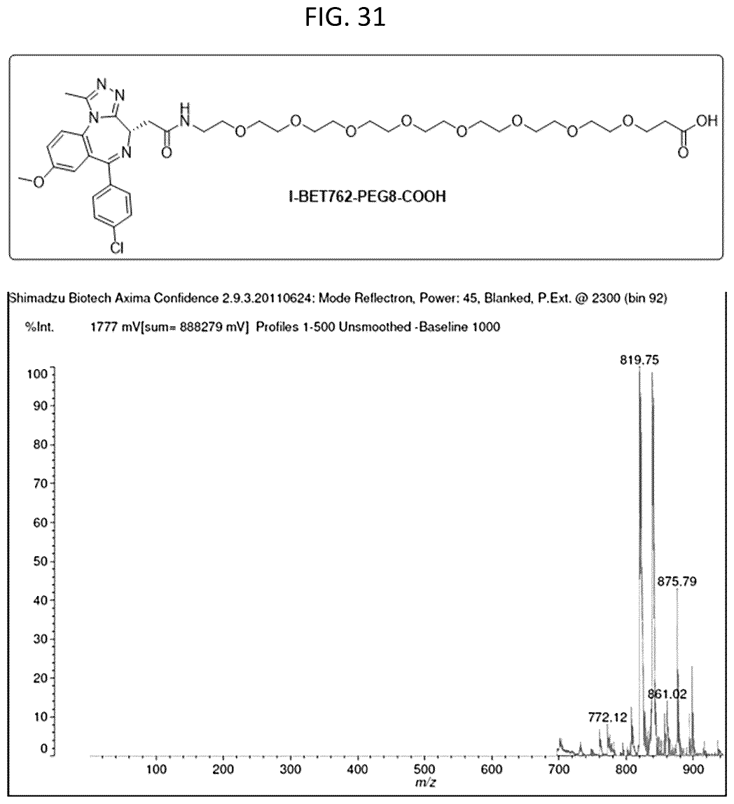

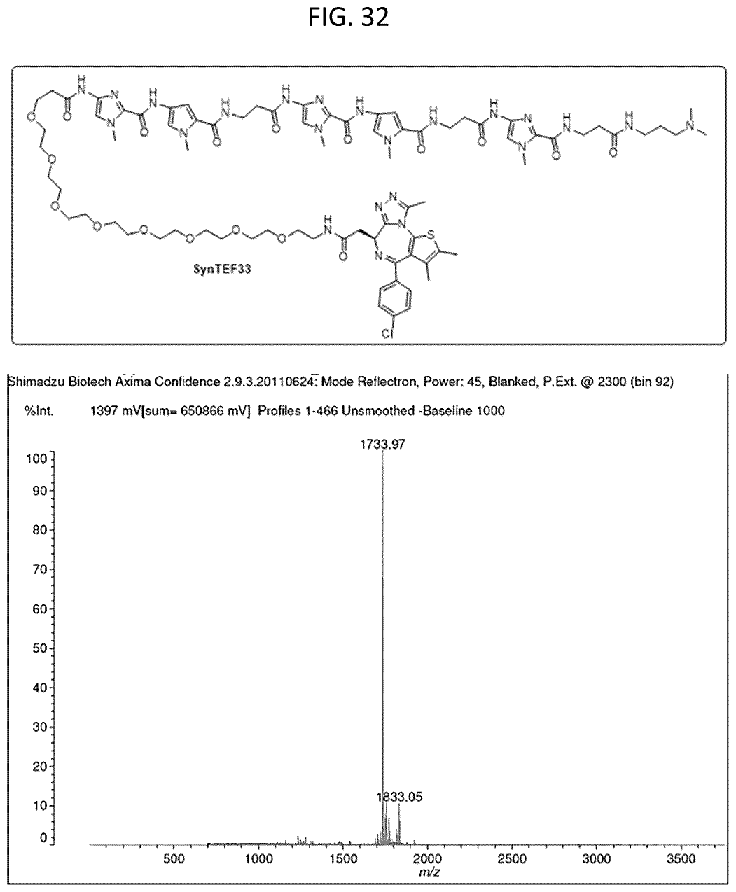

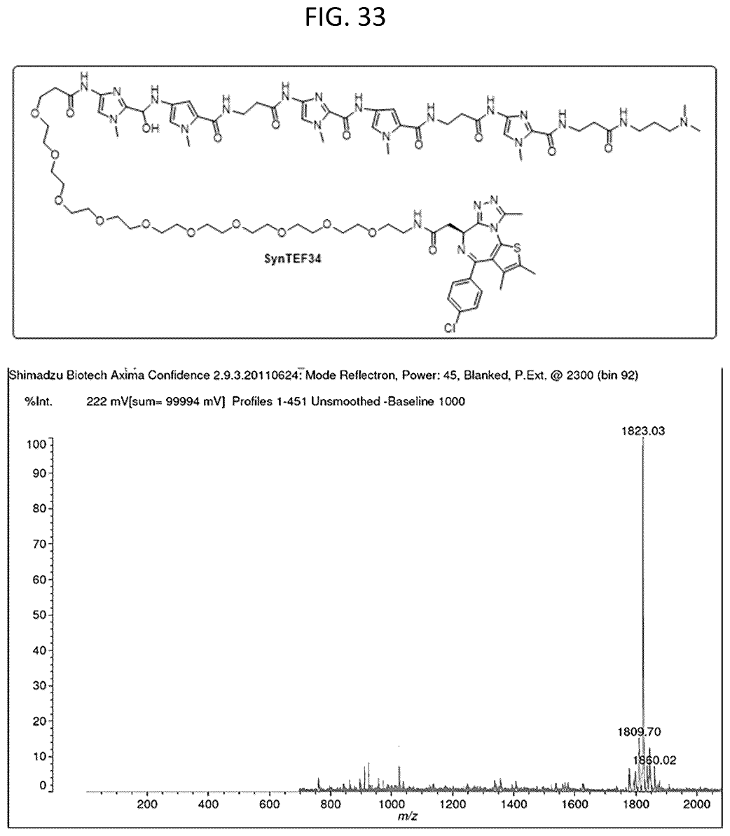

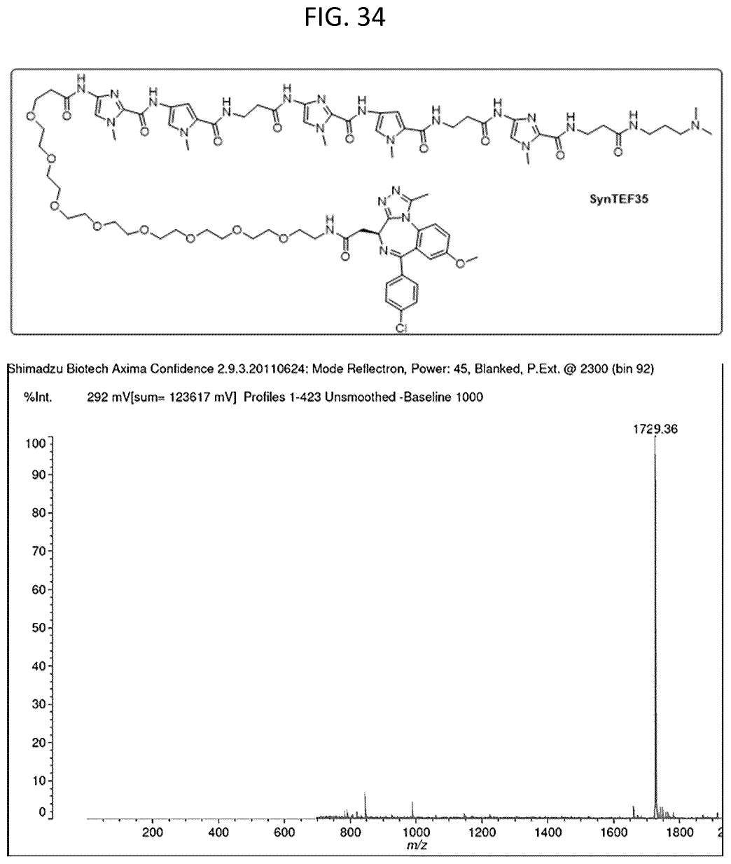

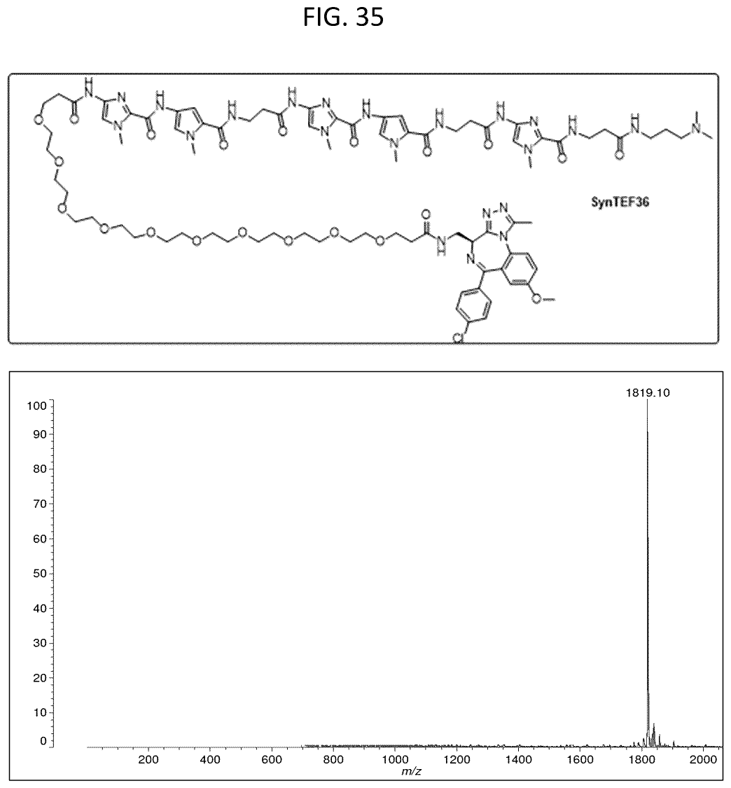

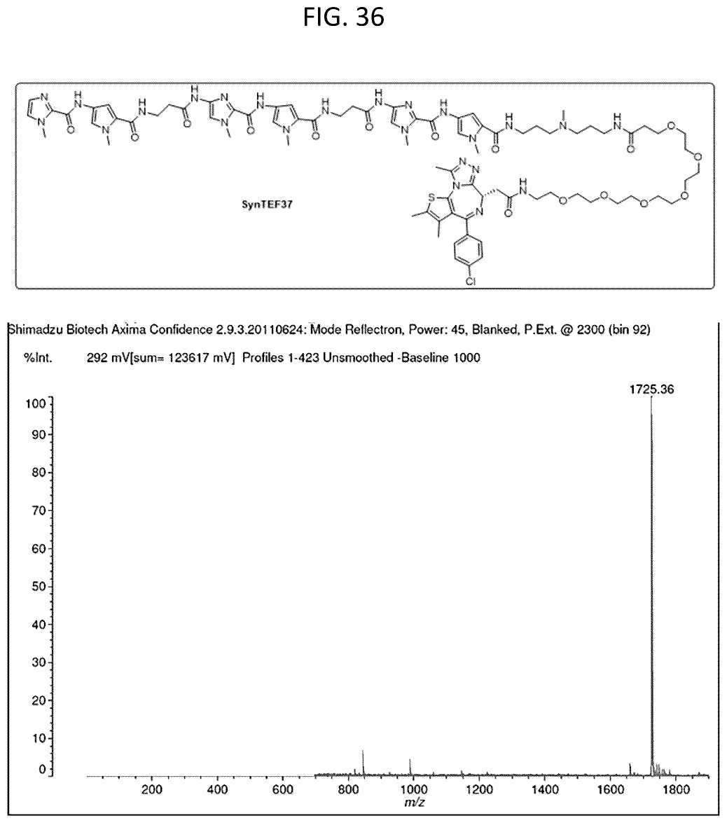

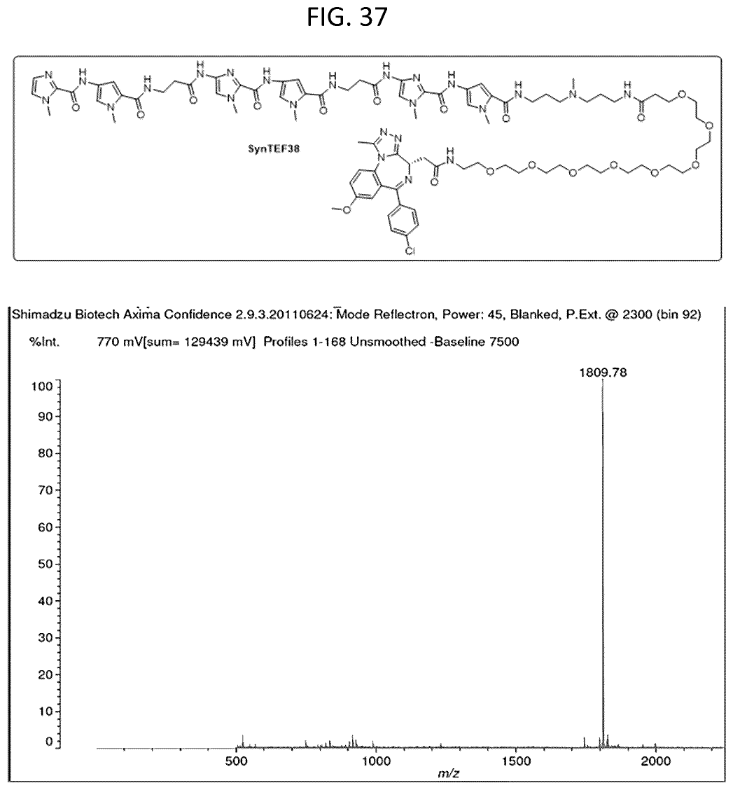

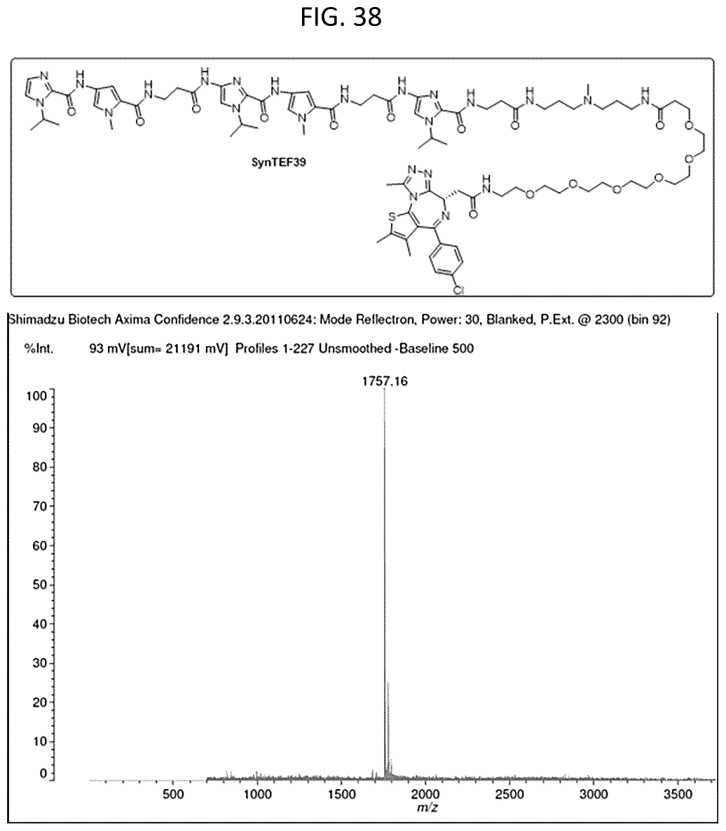

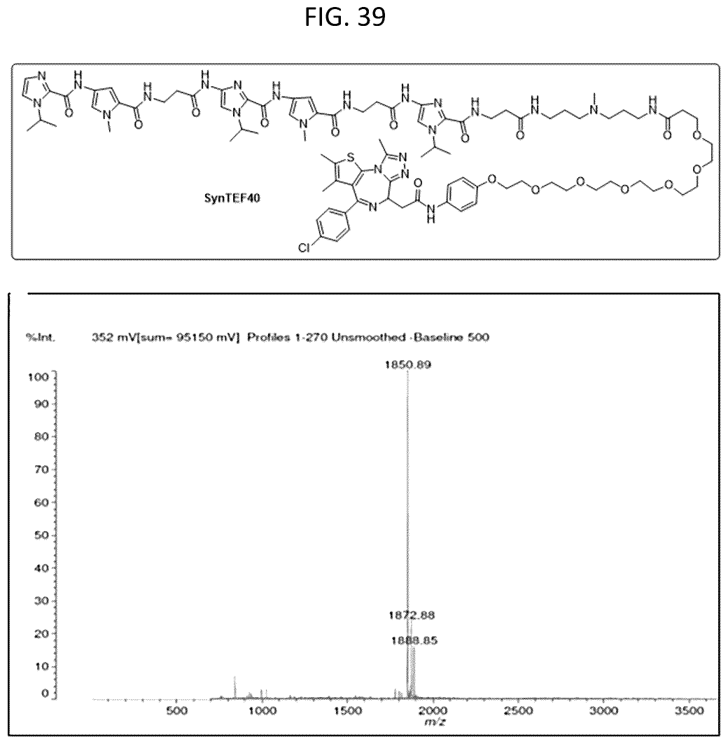

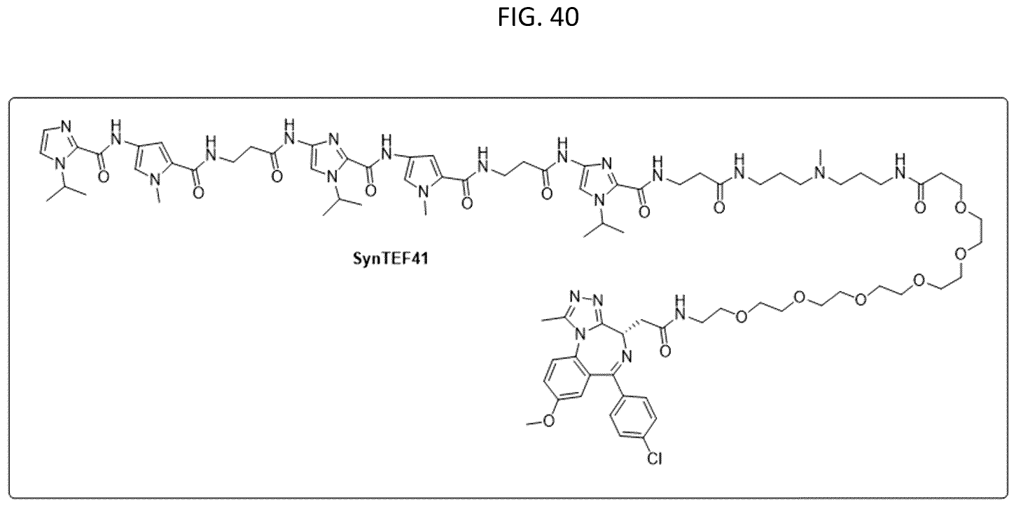

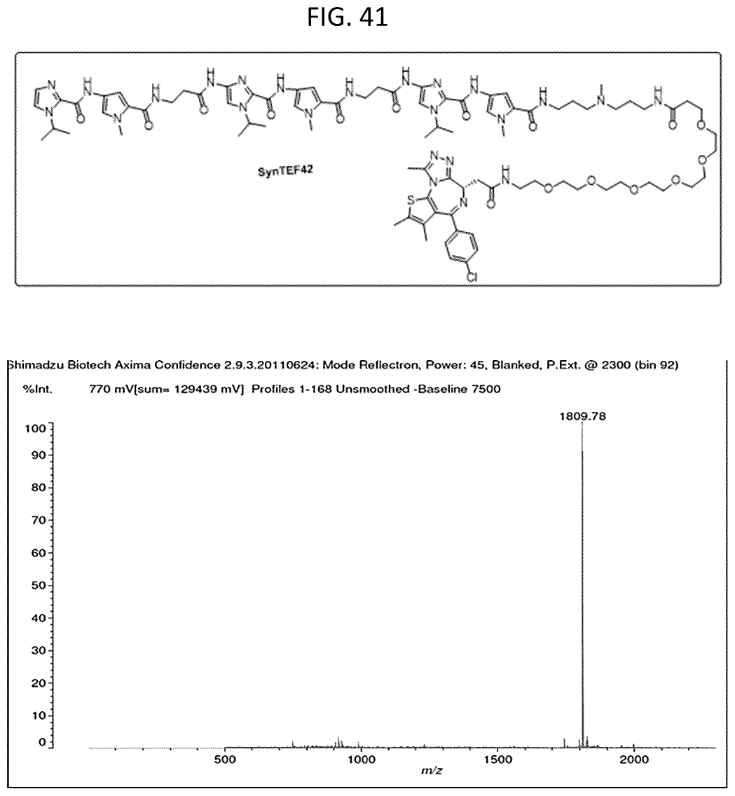

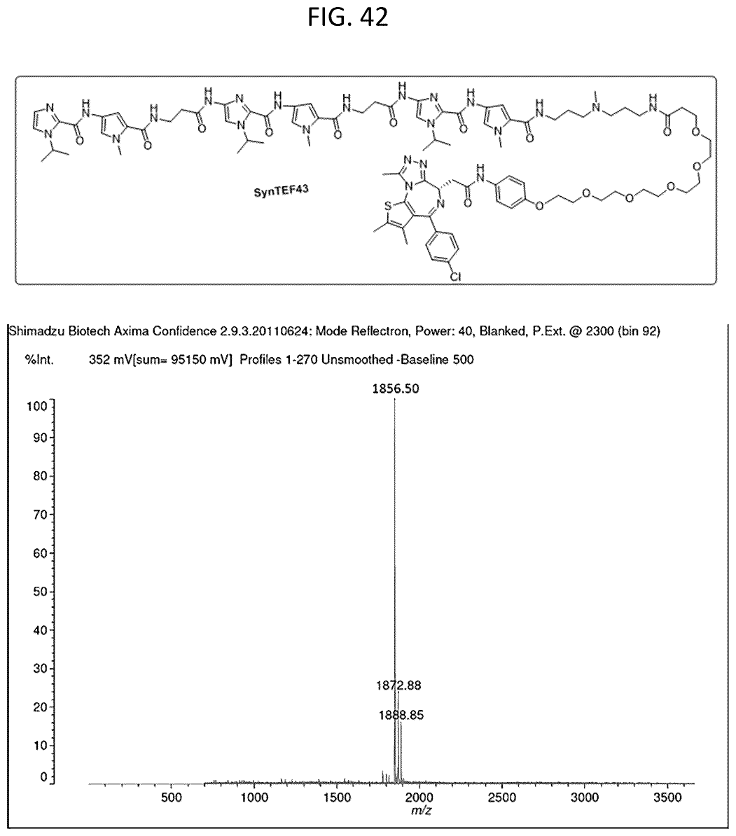

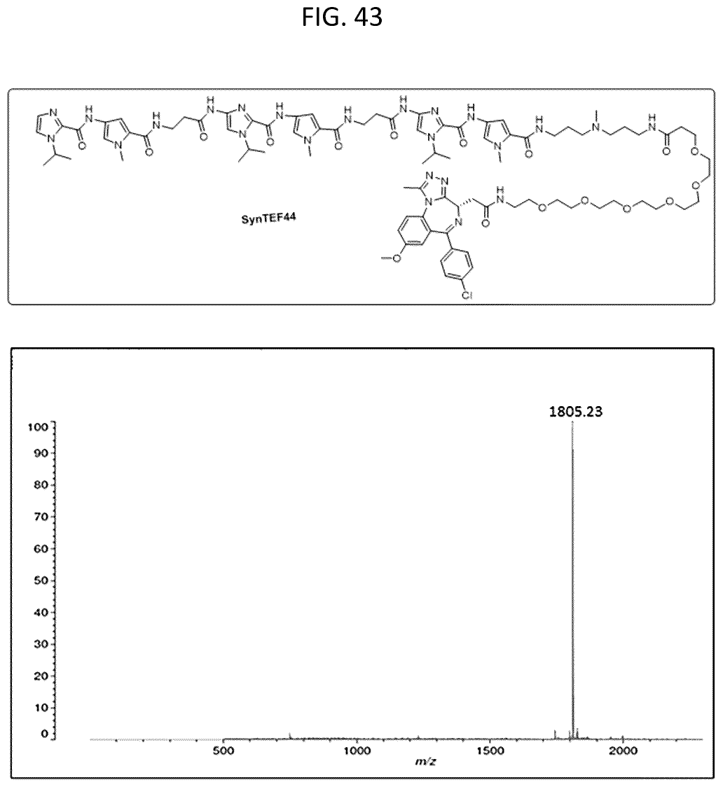

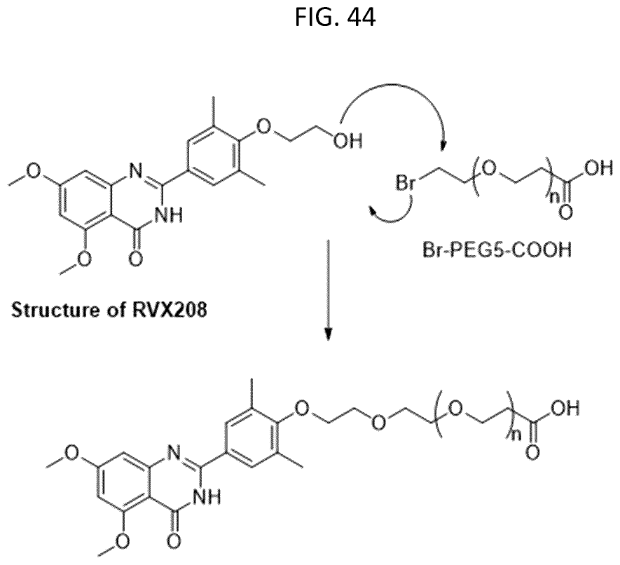

Synthesis of SynTEF4 is shown in Scheme 5 of FIG. 22. For synthesis of OTX-PEG₅-Br, K₂CO₃ (16 mg) was added to a stirred solution of OTX015 (20 mg) in acetonitrile (1 ml) at 0° C. and stirred for 10 min at same temperature. After 10 minutes, the solution of Br-PEG-Br (16 mg) was added to the reaction mixture and then the reaction stirred for 3 days at 60° C. The reaction was monitored by TLC. After completion of the starting materials, reaction mixture was concentrated and purified by HPLC chromatography to give a pure pale yellow compound as a semi solid (10 mg, yield: 30%).

For synthesis of SynTEF4, K₂CO₃ (5 mg) was added to a stirred solution of OTX-PEG5-Br (10 mg) in acetonitrile (1 ml) at 0° C. and stirred for 10 min at same temperature. After 10 min, the solution of PA1 (10 mg) was added to the reaction mixture and then the reaction stirred for 3 days at 60° C. The reaction was monitored by TLC. After completion of the starting materials, reaction mixture was concentrated and purified by HPLC chromatography to give a pure pale yellow compound as a semi solid (0.8 mg, yield: 30%).

### Synthesis of SynTEF5

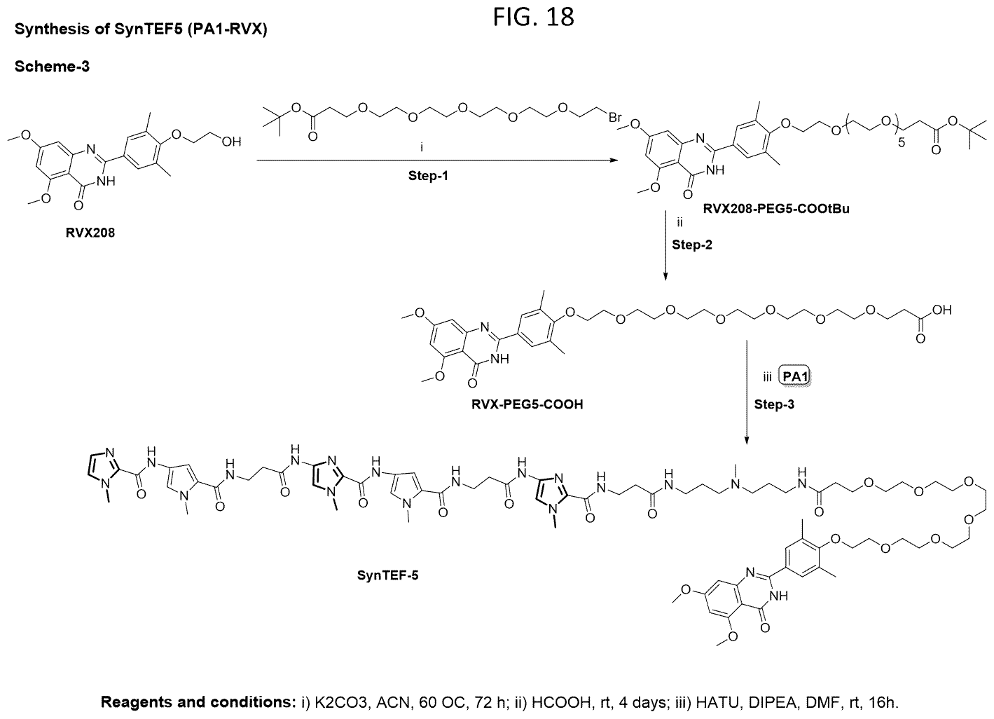

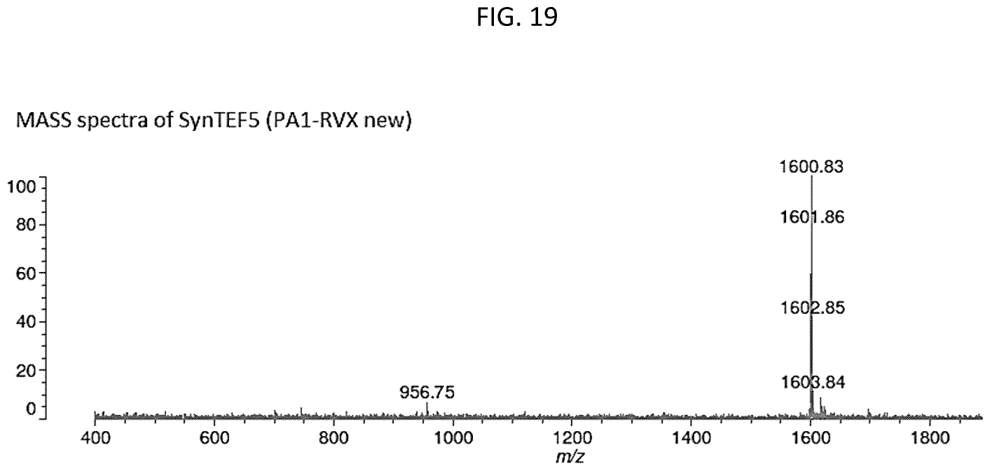

Synthesis of SynTEF5 is shown in Scheme 3 of FIG. 18. For Scheme 3, reagents and conditions are as follows: i) K₂CO₃, ACN, 60 OC, 72 h; ii) HCOOH, room temperature, 4 days; iii) HATU, DIPEA, DMF, room temperature, 16 h. To synthesize RVX-PEG₅-COOtBu K₂CO₃ was added to a stirred solution of RVX208 (50 mg) in acetonitrile (2 ml) at 0° C. and stirred for 10 min at same temperature. After 10 min, the solution of Br-PEG-COOtBu (57 mg) was added to the reaction mixture and then the reaction stirred for 3 days at 60° C. The reaction was monitored by TLC. After completion of the starting materials, reaction mixture was concentrated and purified by HPLC chromatography to give a pure pale yellow compound as a semi solid (40 mg, yield: 41%).

To synthesize RVX-PEG₅-COOH, formic acid (2 ml) was added to a compound RVX-PEG₅-COOtBu (40 mg) at room temperature and the reaction mixture was stirred for four days at room temperature. After completion of the reaction, reaction mixture was concentrated and co-distilled with ether 3 or 4 times to give pale yellow color compound as a liquid (28 mg, yield: 80%).

To synthesize SynTEF-5, PA1 (7.2 mg) and HATU (8.6 mg) were added to a stirred solution of RVX-PEG₅-COOH (5 mg) in DMF (2 ml) followed by DIPEA (4 μL) at room temperature. The reaction was stirred at room temperature for 16 h. After 16 h, reaction mixture was dissolved in 15% acetonitrile in water (0.1% TFA) and purified by reverse-phase prep HPLC on a C18 column. Fractions that showed pure polyamide were frozen in liquid nitrogen and lyophilized to afford a white or off-white powder (3 mg, yield: 25%).

### Synthesis of SynTEF11

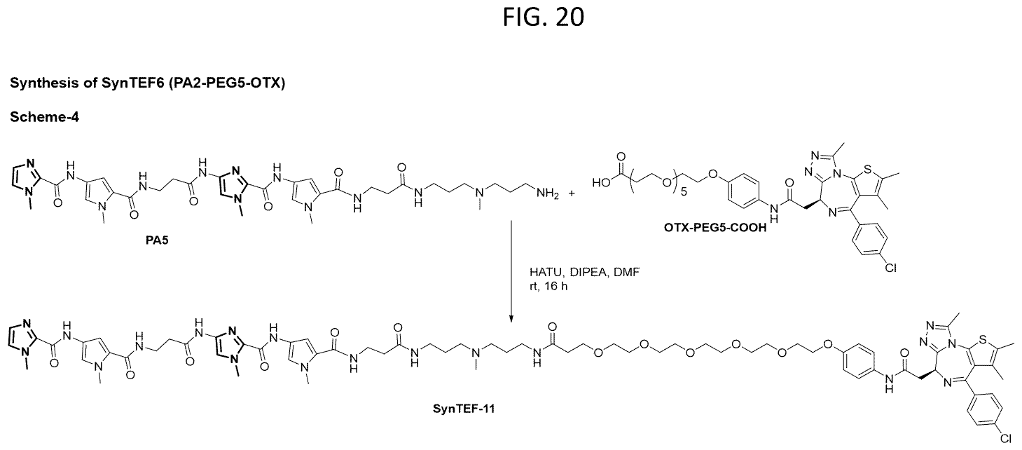

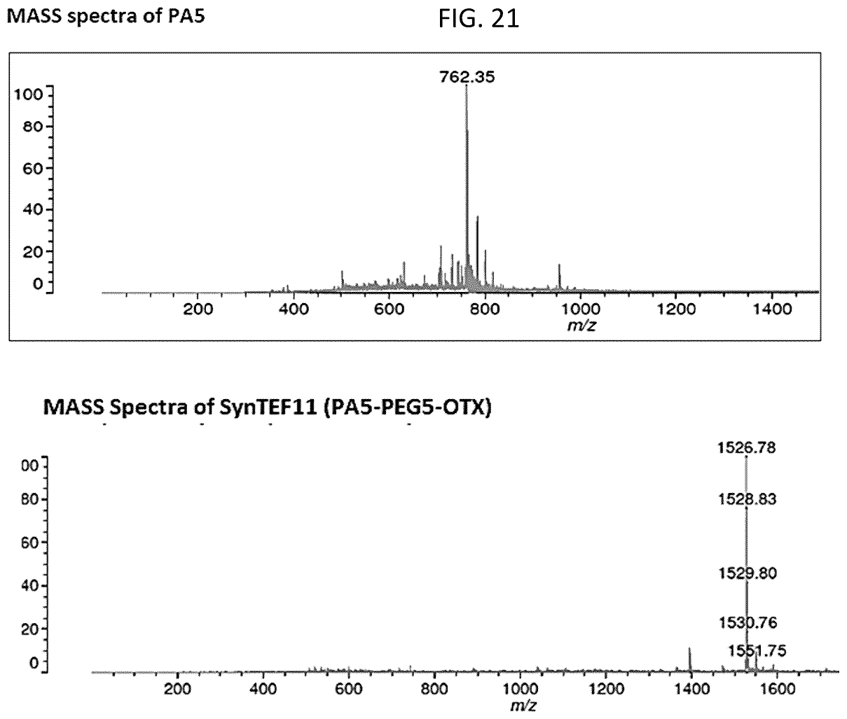

Synthesis of SynTEF11 is shown in Scheme 4 of FIG. 20. The polyamide PA5 was synthesized from Boc-β-Ala-Pam resin using solid phase synthesis protocols. OTX-PEG₅-COOH was synthesized using the synthetic protocols described in FIG. 14 for the synthesis of SynTEF3. For synthesis of SynTEF11, HATU (7.4 mg) and DIPEA (3 μL) were added to a mixture of PA5 (5 mg) and OTX-PEG₅-COOH (5 mg) in DMF (1 ml) at room temperature. The reaction mixture was stirred for 16 h. After 16 h, reaction mixture dissolved in ACN and water and then purified by HPLC to give pure compound as white solid (3 mg, yield: 26%).

### Description of in Vitro FXN Expression Experiments

GM04078 fibroblast cells were exposed to 1 μM (unless indicated otherwise) of the indicated compounds and incubated for 24 hours. RNA was extracted from cells (Qiagen RNeasy kit), and reverse transcribed to obtain cDNA (iScript). qPCR (BioRad SSo SYBR) was performed on the cDNA to amply the FXN target region. Expression was normalized against GAPDH as a house keeping gene. GM04078 (Coriel) fibroblast cells were cultured in MEM medium with 15% FBS.

### EXAMPLE 2

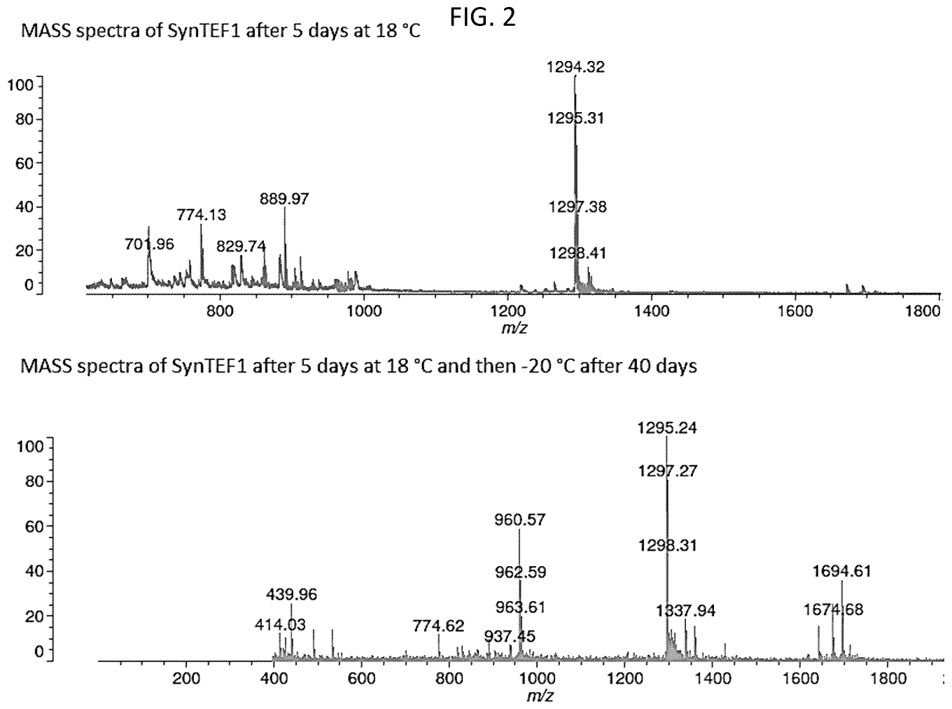

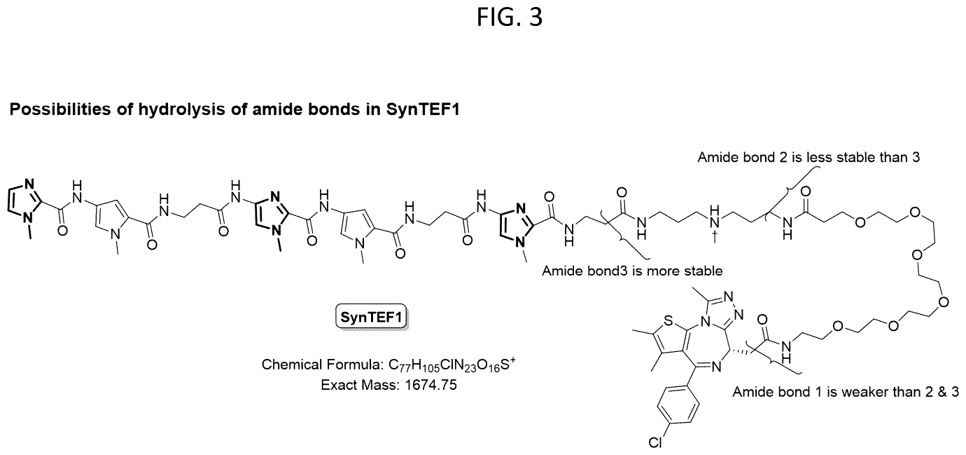

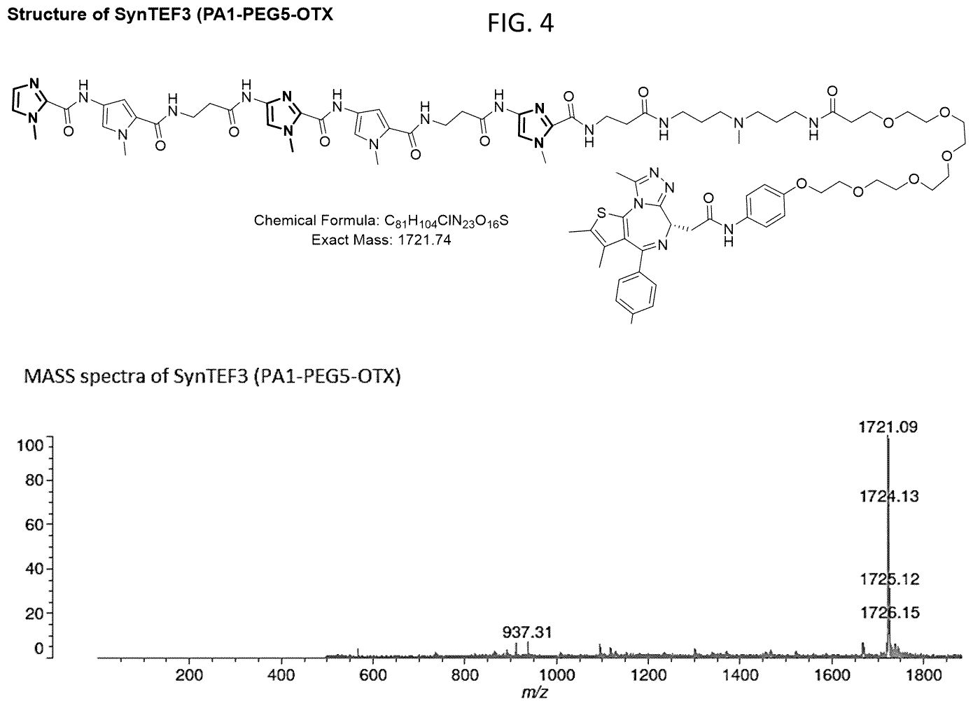

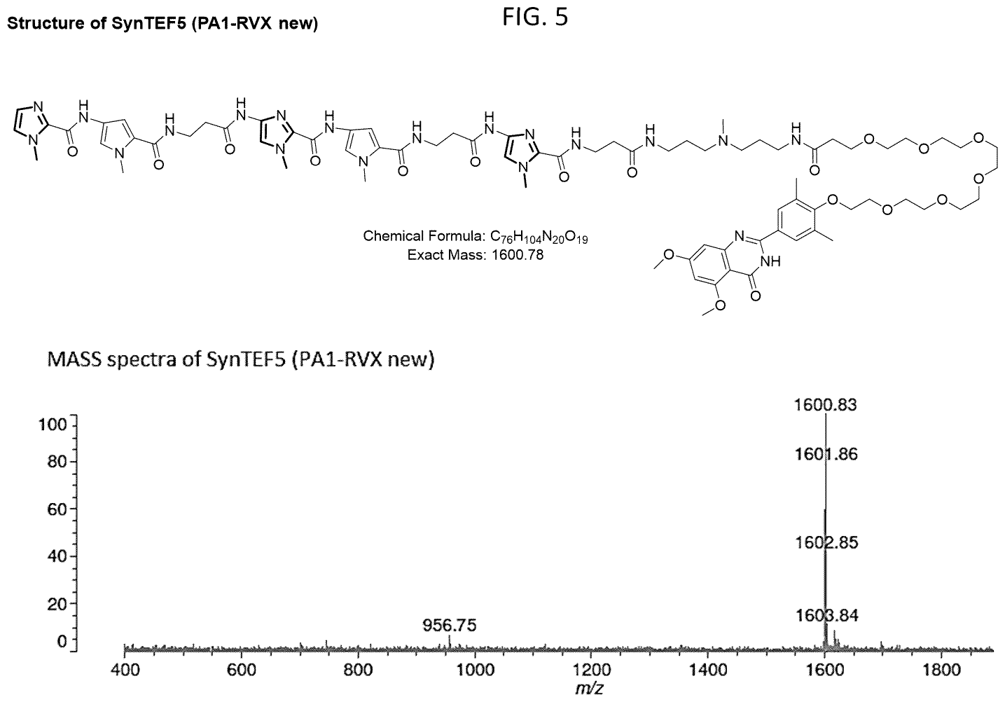

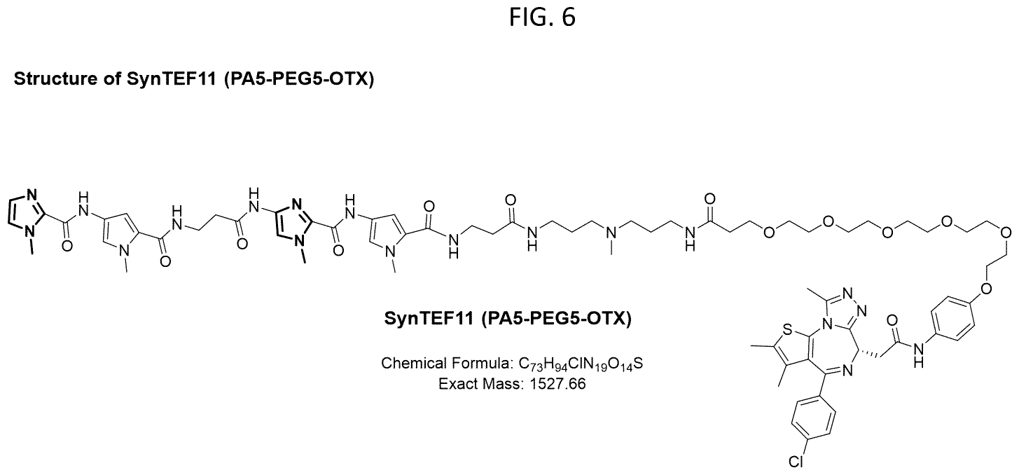

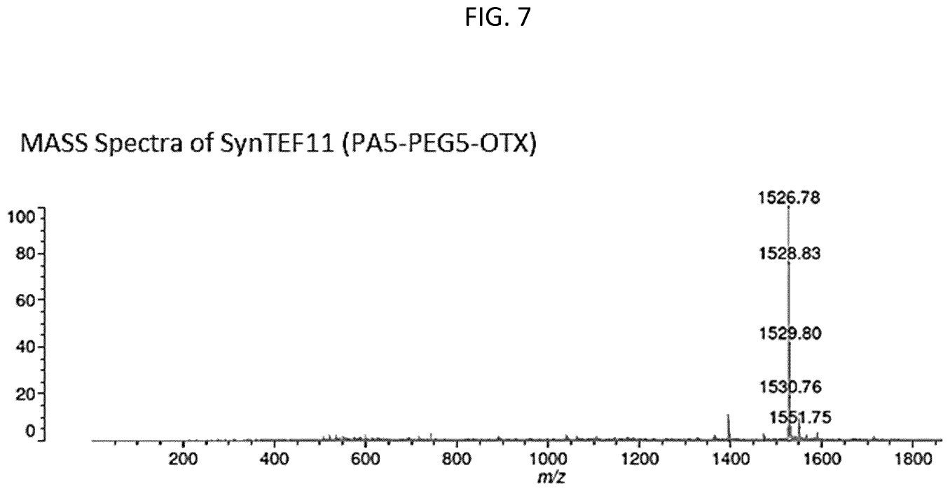

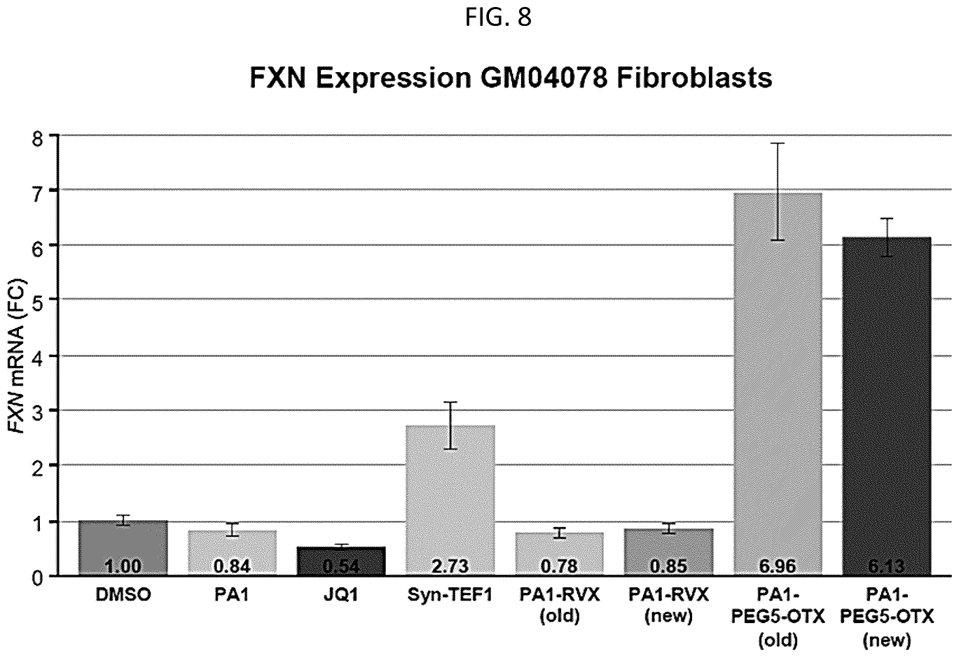

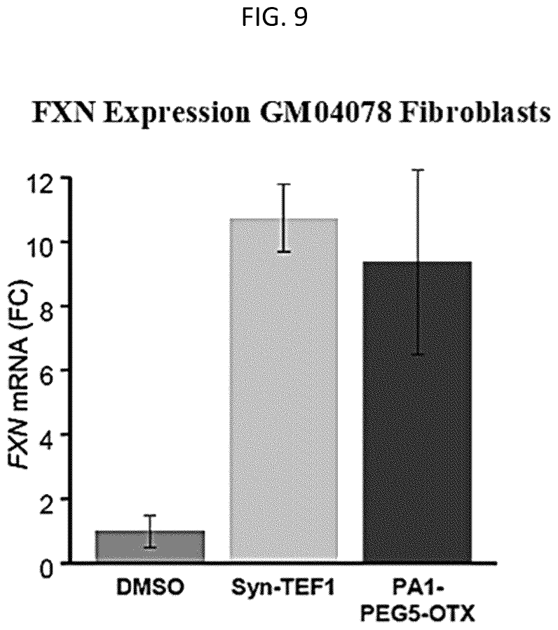

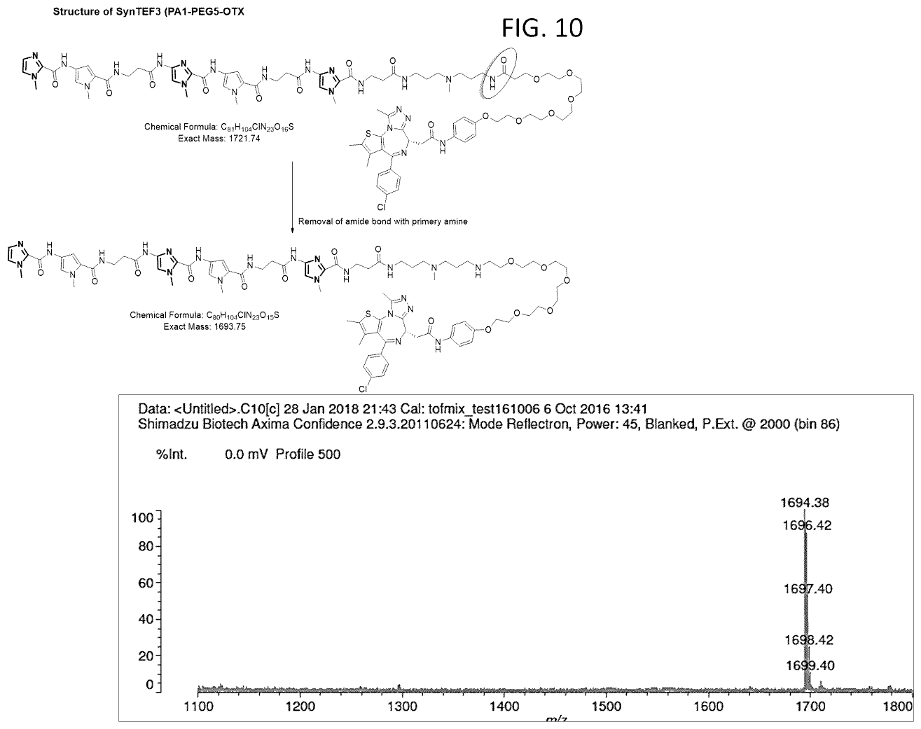

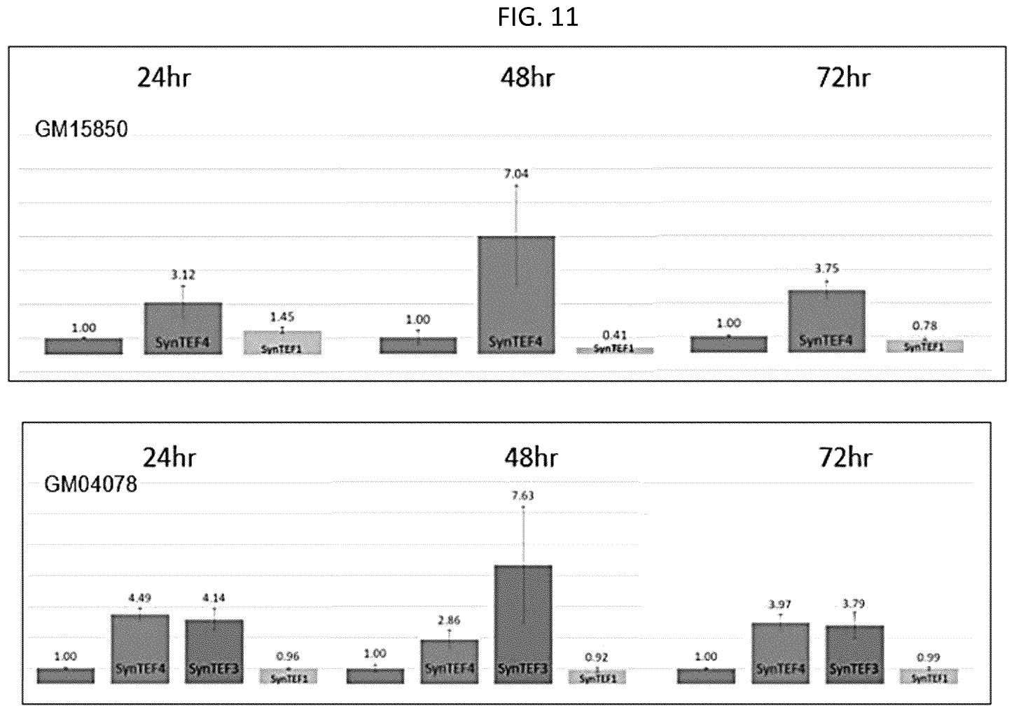

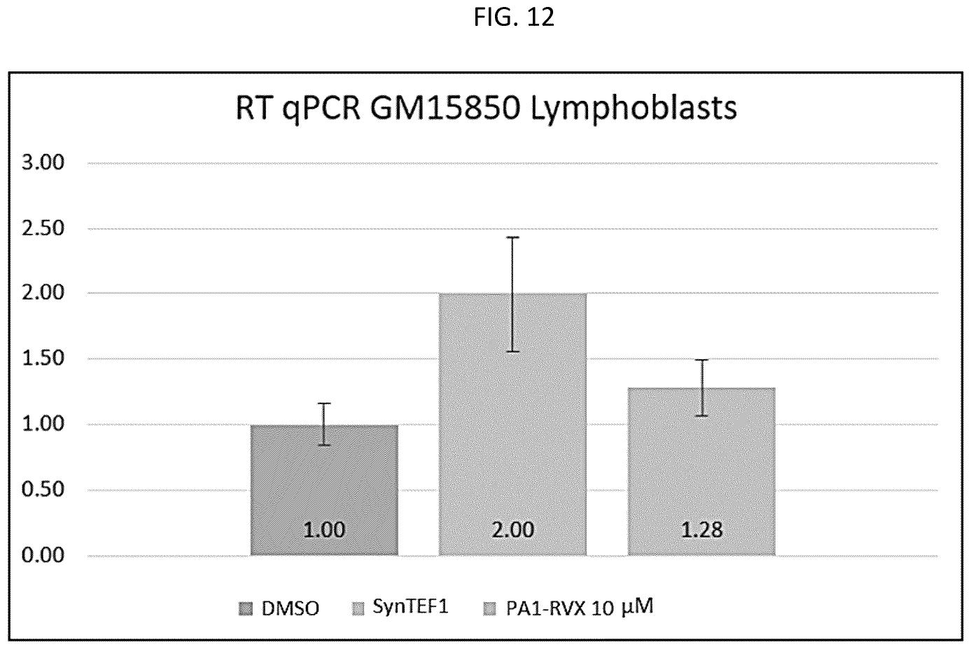

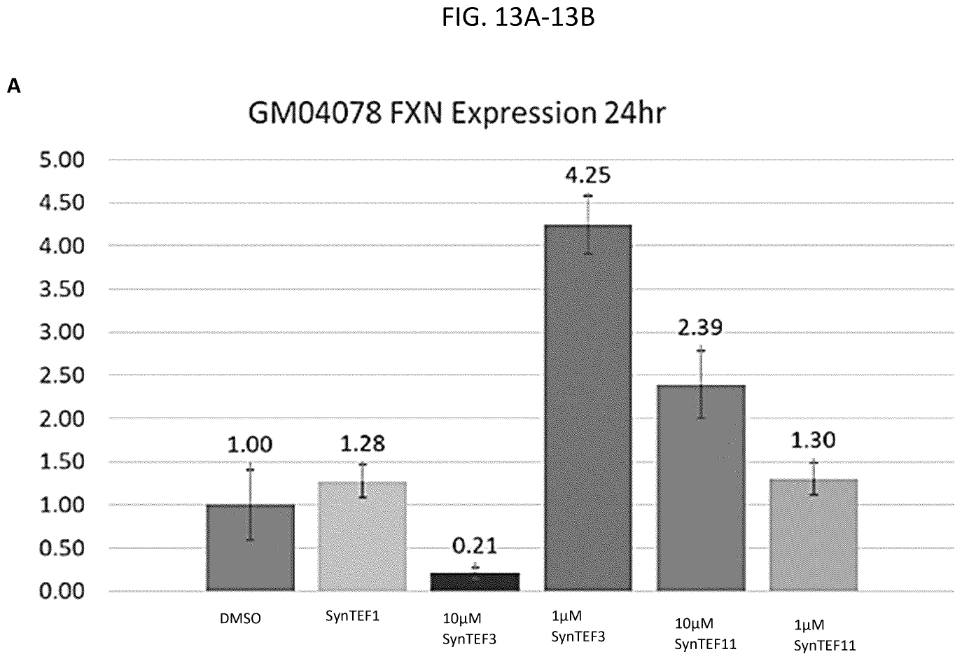

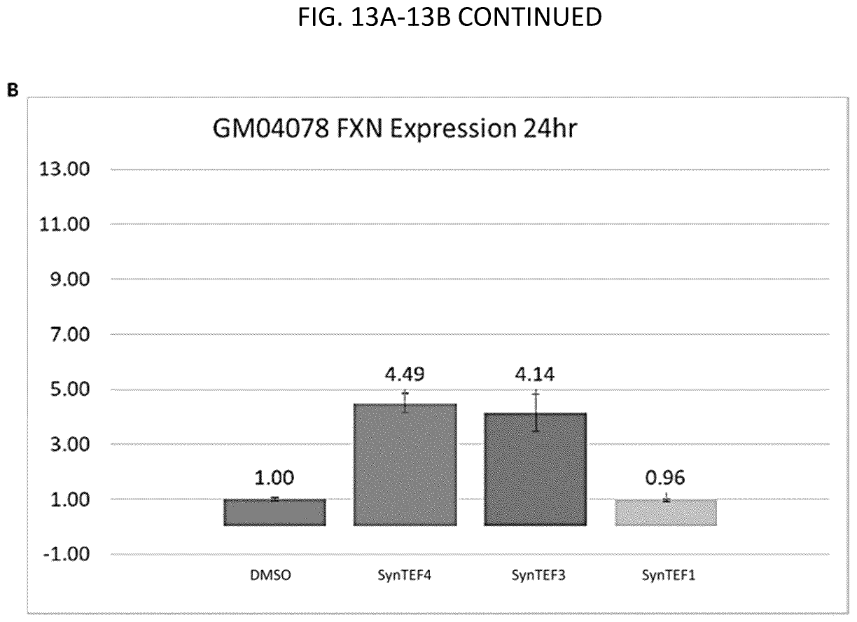

Synthetic transcription factors lacking an amide bond between the A- and -L- moieties were found to have increased stability relative to those that have the amide bond between the A- and -L- moieties. As illustrated in FIG. 3, SynTEF1 includes a hydrolyzable amide bond (referred to as amide bond 2) between JQ1 (“A-”) and the PEG linker (“-L-”). In as little as 5 days at 18° C., SynTEF1 begins to break down (FIG. 2).

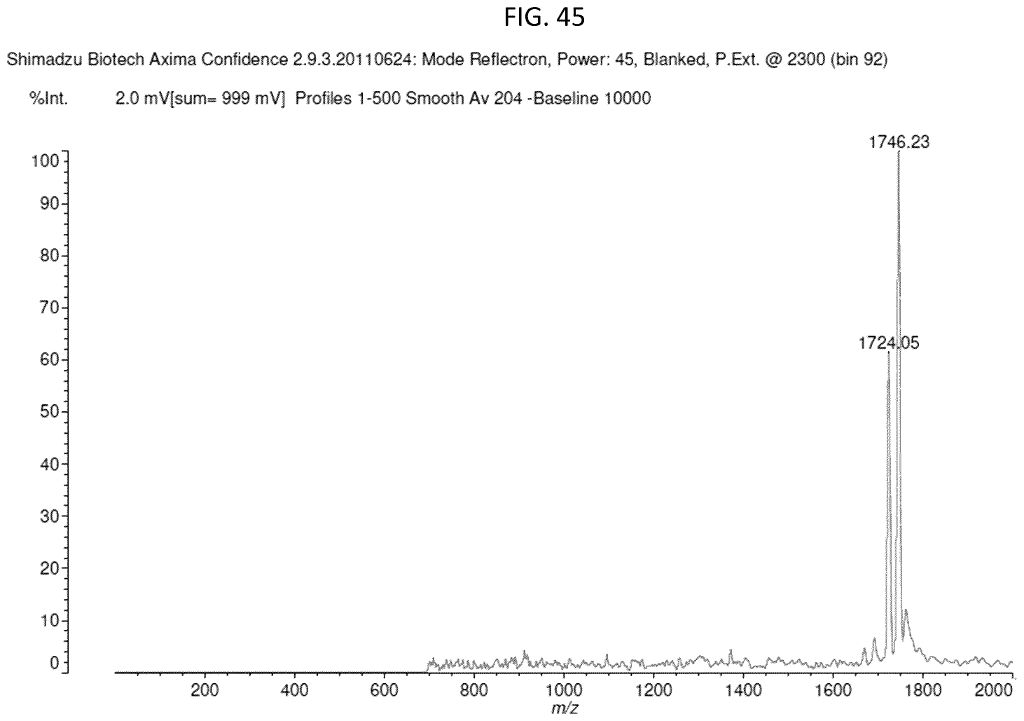

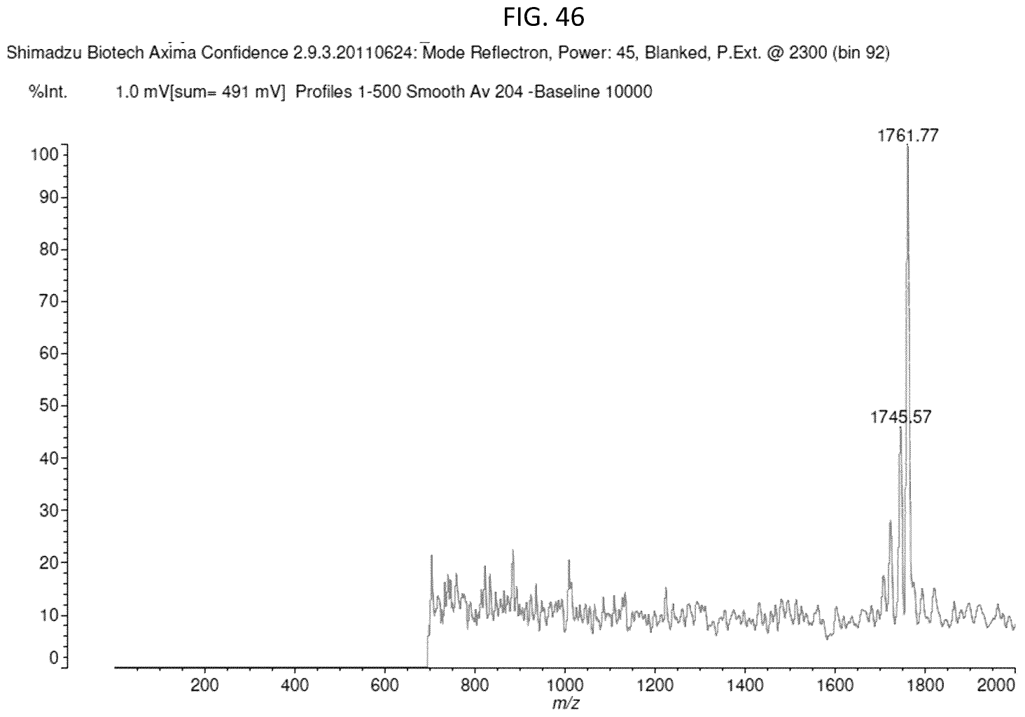

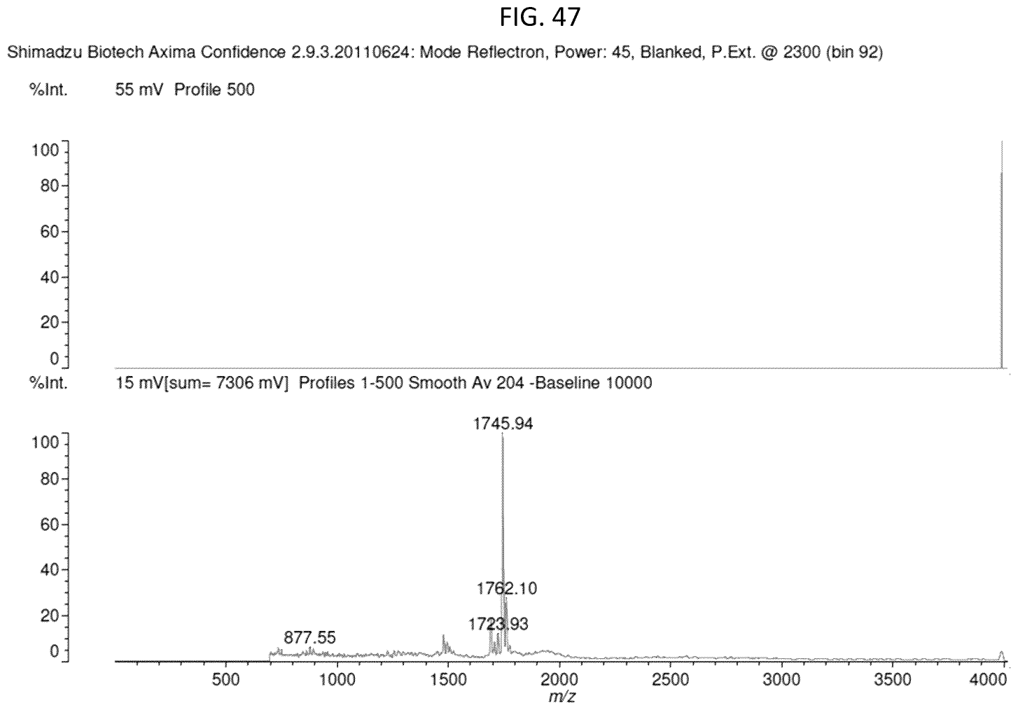

In contrast, SynTEF3, which does not include the problematic amide bond 2 and instead includes an ether bond between A- and -L-, is more stable. As shown in FIGS. 45-47, SynTEF3 does not begin to break down until day 14 at 37° C. This demonstrates that replacement of the amide bond between A- and -L- results in increased stability of the synthetic transcription factor. Other synthetic transcription factors having an ether bond as described herein between the bromodomain binding moiety and the linker also have shown improved stability.

1-76. (canceled)

77. A synthetic transcription factor having formula A-L-B, wherein:
-L- is a linker;
—B is a polyamide that specifically binds to one or more repeats of a GAA oligonucleotide; and
A- is a bromodomain binding moiety having the structure of Formula 9: 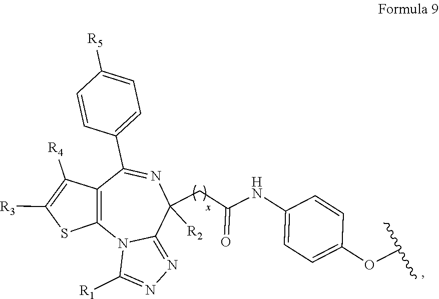
wherein:
R₁, R₃, and R₄ are each independently selected from hydrogen, methyl, ethyl, and halomethyl;
R₂ is selected from hydrogen, C₁-C₆ alkyl group, and substituted C₁-C₆ alkyl group;
R₅ is selected from halogen, aryl, substituted aryl, amino, and amido; and
X is an integer from 1 to 6.

78. The synthetic transcription factor of claim 77, wherein the bromodomain binding moiety has the structure of Formula 10:

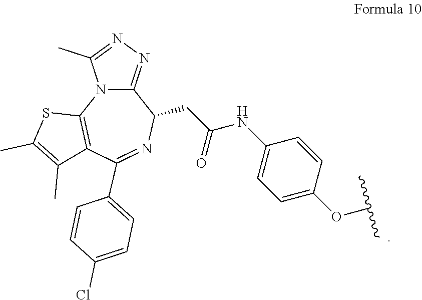 [SMILES: *Oc1ccc(NC(=O)C[C@@H]2N=C(c3ccc(Cl)cc3)c3c(sc(C)c3C)-n3c(C)nnc32)cc1]

79. The synthetic transcription factor of claim 77, wherein —B comprises one or more of the following subunits: 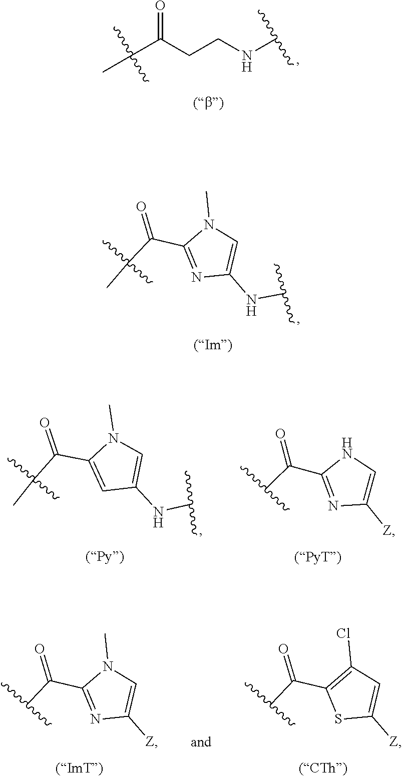
wherein Z is hydrogen, an amino, or an amido group.

80. The synthetic transcription factor of claim 77, wherein —B is —X—(β-Py-Im)ₙ-β-Py-TRM, wherein:
—X— is -β-Im-, -β-Py-, -β-, or a bond;
n is 1-10; and
-TRM is-ImT or -CTh,
with the proviso that one of the -β-Py-Im-trimers may be replaced with a -β-Im-Im-trimer.

81. The synthetic transcription factor of claim 80, wherein n is 1 or 2 and none of the -β-Py-Im-trimers are replaced by a -β-Im-Im-trimer.

82. The synthetic transcription factor of claim 80, wherein n is 1 or 2 and one of the -β-Py-Im-trimers is replaced by a- β-Im-Im-trimer.

83. The synthetic transcription factor of claim 80, wherein —B is —(β-Py-Im)ₙ—(β-Py-ImT), wherein Z is hydrogen and n is 1 or 2.

84. The synthetic transcription factor of claim 80, wherein —B is -β-Py-β-Py-Im-β-Py-ImT, -β-β-Py-Im-β-Py-ImT,-β-Im-β-Py-Im-β-Py-Im-β-Py-ImT, -β-Py-β-Py-Im-β-Py-Im-β-Py-ImT, -β-Py-Im-β-Py-Im-β-Py-ImT, -β-Py-β-Py-Im-β-Py-CTh,-β-β-Py-Im-β-Py-Im-β-Py-ImT, or -β-Py-Im-β-Py-CTh.

85. The synthetic transcription factor of claim 77, wherein —B is

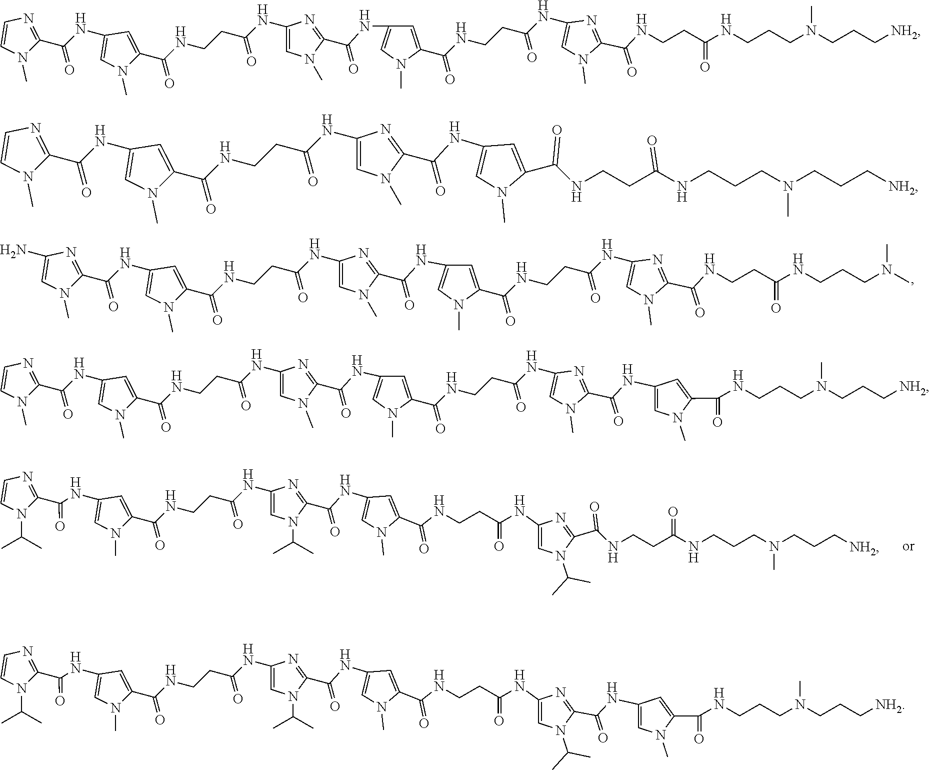

86. The synthetic transcription factor of claim 77, wherein the linker comprises a linking moiety selected from the group consisting of —O—, —(CH₂)ₓ—, —(CH₂CH₂)_{y}—, —C(O)NR′—, —NR′C(O)—, —C(O)—, —NR*—, and 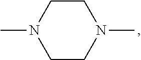
wherein R′ and R* are each independently a hydrogen or C₁-C₆ alkyl, and x and y are each independently an integer from 1 to 10.

87. The synthetic transcription factor of claim 77, wherein -L- includes —(CH₂)ₓ—C(O)N(R′)—(CH₂)_{Q}—N(R*)—(CH₂)_{Q}—N(R′)C(O)—(CH₂)ₓ—C(O)N(R′)—, —(CH₂)ₓ—C(O)N(R′)—(CH₂CH₂O)_{y}—(CH₂)ₓ—C(O)N(R′)—, —C(O)N(R′)—(CH₂)_{Q}—N(R*)—(CH₂)_{Q}—N(R′)C(O)—CH₂)ₓ—, —(CH₂)ₓ—O—(CH₂CH₂O)_{y}—(CH₂)ₓ—N(R′)C(O)—(CH₂)ₓ—, or —N(R′)C(O)—(CH₂)ₓ—C(O)N(R′) —(CH₂)ₓ—O—(CH₂CH₂O)_{y}—(CH₂)ₓ—, wherein R* is methyl, R′ is hydrogen, Q is an integer from 2 to 10, and x and y are independently an integer from 1 to 10.

88. The synthetic transcription factor of claim 77, wherein -L- is a polyethylene glycol (PEG) linker.

89. The synthetic transcription factor of claim 88, wherein -L- is a PEG linker selected from the group consisting of (O—CH₂—CH₂)₂, (O—CH₂—CH₂)₃, (O—CH₂—CH₂)₄, (O—CH₂—CH₂)₅, (O—CH₂—CH₂)₆, (O—CH₂—CH₂)₇, (O—CH₂—CH₂)₈, (O—CH₂—CH₂)₉, and (O—CH₂—CH₂)₁₀.

90. The synthetic transcription factor of claim 77, wherein the synthetic transcription factor has a non-amide bond between A- and -L-.

91. The synthetic transcription factor of claim 77, wherein the synthetic transcription factor has an ether bond between A- and -L-.

92. The synthetic transcription factor of claim 77, wherein the synthetic transcription factor has a non-amide bond between -L- and —B.

93. The synthetic transcription factor of claim 77, wherein the —B and -L- moieties are joined by an amide bond.

94. The synthetic transcription factor of claim 77, wherein the synthetic transcription factor is

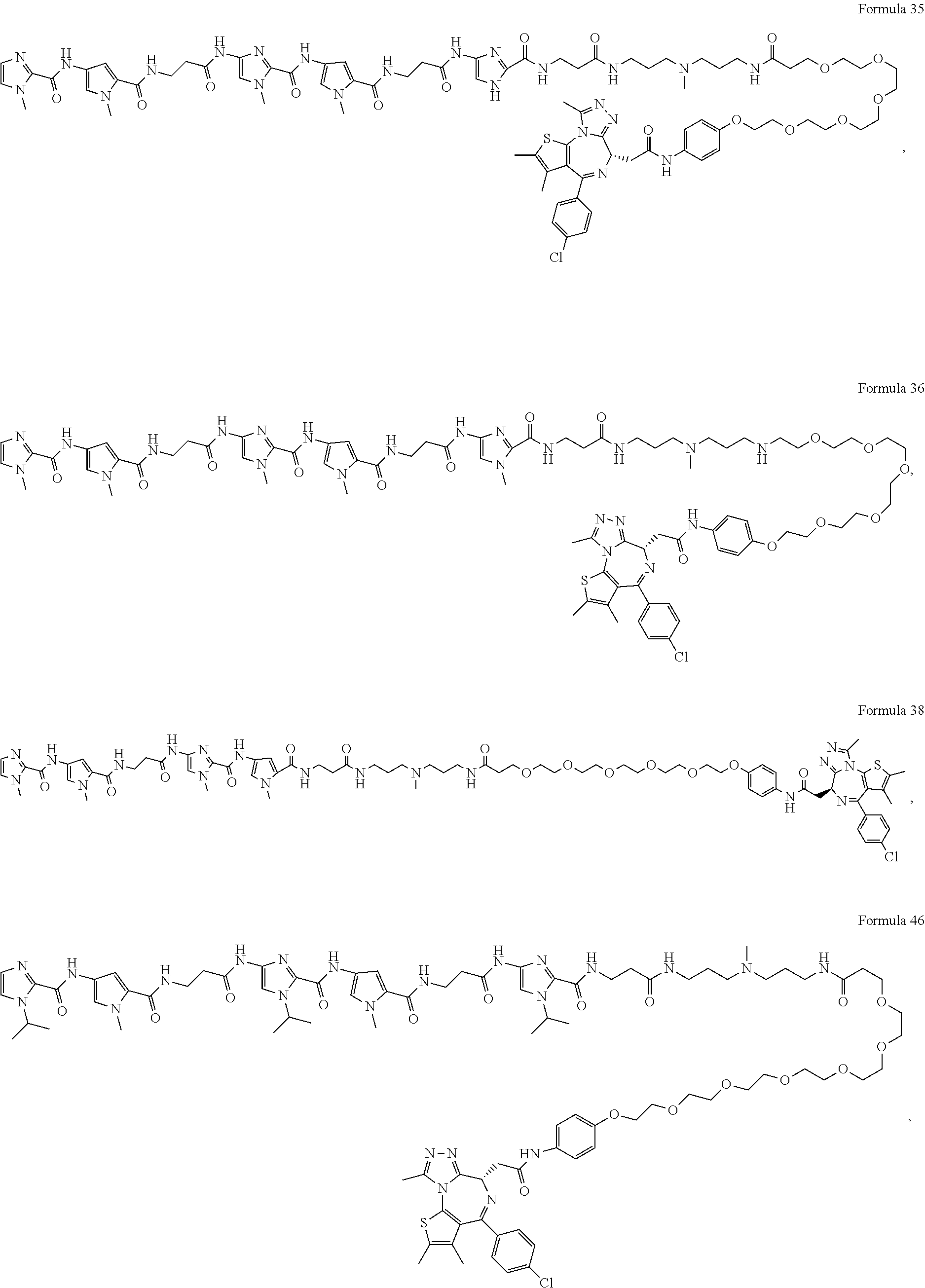

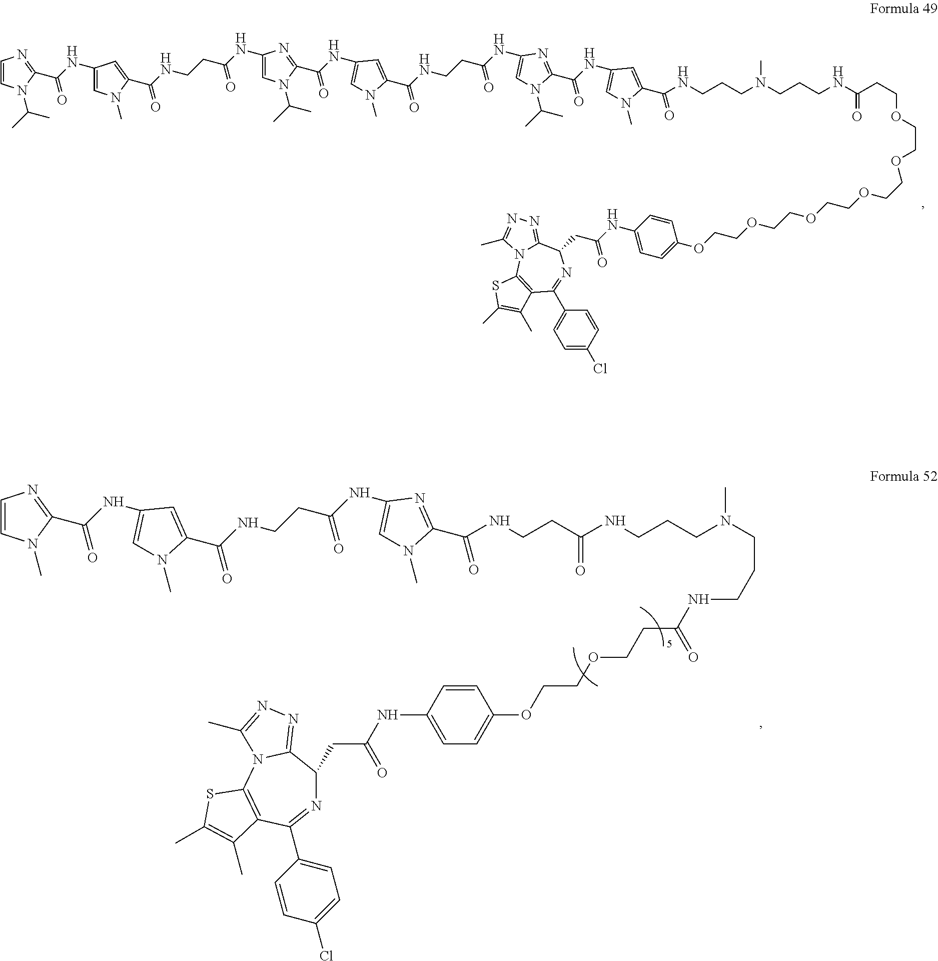

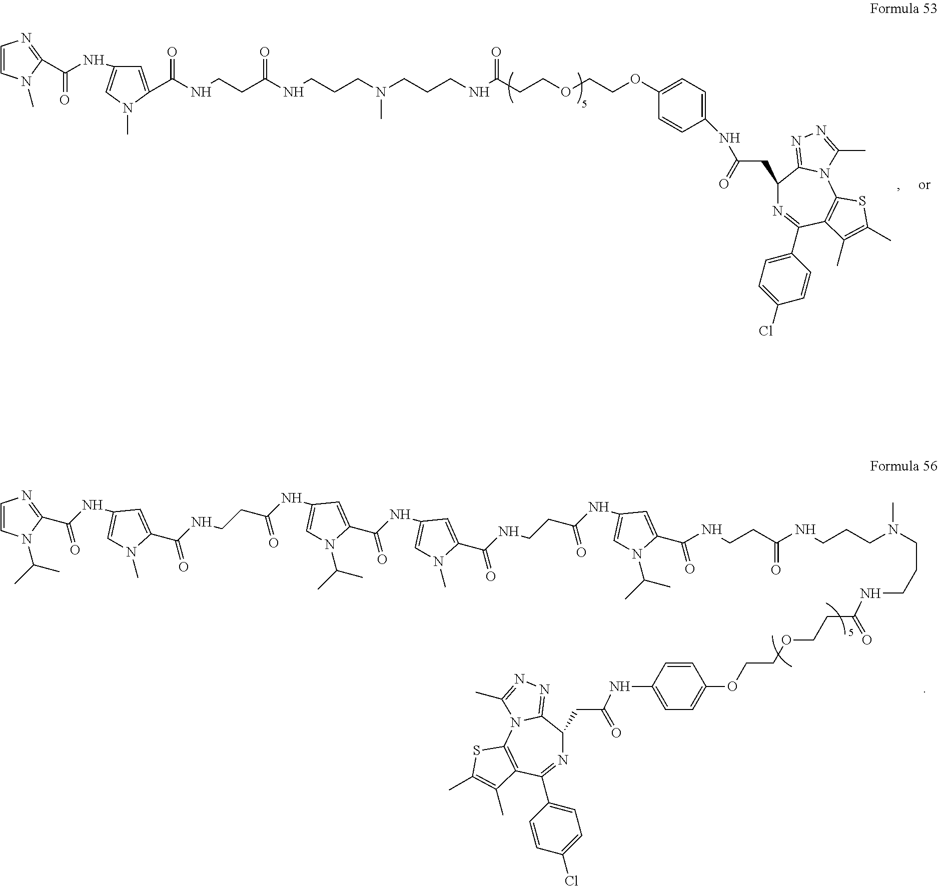

95. A pharmaceutical composition comprising the synthetic transcription factor of claim 77, and a pharmaceutically acceptable carrier.

96. A method for treating Friedreich's ataxia in a subject in need thereof, comprising administering to the subject a therapeutically effective amount of the composition of claim 95.
